SEQUENCE LISTING (17 sequences)

SEQ ID NO: 1 - DNA, 1110 bp, Mus musculus
ggatccacggcctctggccgcattttaccactgagctacactcccaaagcagtcgaaatcacagtggcccaggattgaaatgatcacttagatgctttgcagtcttgataagacactaaatctttgtctatcagttacttcatctttaataacagaacgtacttaggaattttatgagcattgttagttagcatgacacatgctatatgtattcgtcattatgaataatgtaaccacagcaattacattgtactttttattataaaaggggggaggggaaggccctggtccttttttaacttctgagaggtttcgattactaagtaagaccttatgtagacttccatttgggagctgagaaagcagaggattccaaaaggggatgacatttgcaaaggtctagaaaaggcgcctgggaattctaccgggtaggggaggcgcttttcccaaggcagtctggagcatgcgctttagcagccccgctggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctcgcacgcttcaaaagcgcacgtctgccgcgctgttctcctcttcctcatctccgggcctttcgacctcacggtgttgccaaaatgtcgctttccaacaagctgactttggacaagctggacgtgaaggggaagcgggtcgtgatgaggtaattccgtactgctgccctcaagccctcggggccacattctctctggcgtggcaagcacggttttcccatcaccttaagttgcacttatttttcagct

SEQ ID NO: 2 - DNA, 7835 bp, Encephalomyocarditis virus
ttgaaagccgggggtgggagatccggattgccagtctgctcgatatcgcaggctgggtccgtgactacccactccccctttcaacgtgaaggctacgatagtgccagggcgggtactgccgtaagtgccaccccaaaataacaacagaccccccccccccccccccccccccccccccccccccccccccccccccccccccccccccccccccccccccccccccccccccccccccccccccccccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggcaattccacctcctcagacaagaataactcctcctcggaaggcaatgaaggtgtgatcatcaataacttttactccaaccaatatcaaaactccattgacctctctgctaatgcagccgggtctgacccacccagaacctacggtcaattttcgaatctcttttcgggcgcagtgaatgccttttctaatatgcttccattgctagctgatcaaaatacagaagaaatggagaatctgtctgatcgagtgtctcaagacactgccggcaatacggtcacaaacacccagtcaacagtgggccgtcttgtcggttatggtaccgttcatgatggagagcatccggcatcatgtgctgacactgcttcagaaaagatcctggcggtggaaaggtactacaccttcaaggttaatgattggacatcaacacaaaagccctttgagtacatccgcattccccttcctcacgtcctgtccggtgaagatggtggtgtctttggtgcggccctccgccggcactacctggtgaaaactggatggcgggtgcaagttcagtgcaacgcctctcaattccacgctggaggtttgctggtgttcatggcaccagagtatccaaccttagatgcttttgccatggacaaccgttggtccaaggataacctgcctaatggaaccagaactcagacaaacaaaaagggaccatttgccatggaccatcagaacttctggcagtggaccttgtatccccatcaattcctgaatctgagaactaacaccacagtggatcttgaggtgccatatgtaaacatagcccccacttcctcctggacacaacatgcttcctggactttggtgattgcagtggttgctcccctgacatactcaaccggggcttctaccagtttggatatcaccgcttctattcagccagtaaggcctgtctttaatggcctccggcatgagacactttctagacagtcgcccattccggtcacaattagagaacatgctggtacctggtattctactctgccagacagcacagtgcctatttatggcaagactcctgttgctccatccaattacatggtaggcgaatacaaggacttcctggagatagctcagattccaacctttattgggaataagatccctaatgctgtcccctacattgaggcatccaacacagccgtcaagacccaaccgctggccacctatcaagtgaccttgtcctgctcctgtctggccaatacattcttggccgctttgtctagaaactttgctcagtaccggggatcattggtttatacctttgtgttcactgggaccgcgatgatgaagggcaagttcctcattgcctacaccccacctggagcgggcaagcccactagtcgagaccaagccatgcaggcgacttatgcgatttgggatttggggctaaattcttcttactccttcactgtgccttttatttctcccactcacttccgcatggtaggtactgaccaagtcaacatcactaatgcggatggctgggttaccgtgtggcagctcactcccctcacttacccaccaggatgcccgacctctgctaagatactaacaatggtgagcgcagggaaggatttctcactcaagatgcctatctcacctgccccctggagccctcagggagtagaaaacgctgaaaaaggggtcactgaaaacacaaacgcaactgctgactttgtggctcaaccagtttacttgcctgagaaccaaacgaaggtggctttcttctataataggtccagtcccattggtgccttcaccgtgaagtccggcagtctagaatctggttttgccccgttctctaatgggacttgcccgaactcagtgatactgacccctgggccccaatttgaccccgcctatgaccaactcaggccacagcgtctgacagaaatttggggcaatggaaatgaggagacctcaaaagtctttccgcttaaatccaaacaggattattccttctgcctcttctccccctttgtgtattataaatgtgatttagaagtgactcttagtcctcacacttcaggcaaccatgggctgttggtgaggtggtgtcccactggtacaccaaccaagcccactacccaggttctccatgaagtaagttccctctcagaaggcagaaccccccaggtttatagtgccggacctggcatttcaaatcagatttcctttgtagttccttacaattctccactttcagtcctatcagctgtctggtataatggacacaagagatttgacaacactgggagcttgggcattgcccctaattctgatttcggcactctgttctttgctggcacaaagcctgacattaaattcacagtctacttgagatacaagaataagagagttttttgcccacgtccgactgtctttttcccctggcccacttccggagacaagattgatatgaccccgagagctggagtcttgatgctagagagtccaaatgccctagacatttcaagaacataccccacgttacatgttctcattcaattcaaccatagaggtttggaggttagattgtttagacatggacacttttgggctgaaacacgtgcggacgtgattctgagatcaaagaccaaacaggtctctttcctgagcaacgggaactacccgtcaatggactctagagctccctggaatccttggaagaatacctaccaggcggttctaagagcagaaccatgtagagtgaccatggatatatattataagagagtcaggccttttagactgcccctggttcagaaggaatggcccgtgcgagaggagaacgttttcggtttgtaccggatcttcaatgcccactacgctggttactttgcggacctactgattcatgacattgagacaaatccagggcccttcatgtttagaccaaggaaacaggttttccagacccaaggagcggcagtgtcatcaatggctcaaaccctactgccgaacgaccttgccagcaaagctatgggatcagcttttacggctttgctcgatgccaacgaggacgcccaaaaagcaatgaagattataaagacattaagttctctatcggatgcatgggaaaatgtaaaagaaacactaaacaacccagagttctggaagcagctcttgagcagatgtgtgcagctgattgcagggatgacaatagcagtgatgcatccggaccctttgactctgctctgcttaggaacattgacggccgccgagattacaagccagacaagtctgtgcgaagaaatagcagctaagttcaagacaattttcatcactcctccaccacggtttcccacaatctctcttttccaacaacaatcccccttgaaacaggtaaatgatattttctccctagccaagaacctggactgggccgtcaagactgtggaaaaggtggttgattggtttgggacatggatagtacaggaggaaaaggaacagaccctagatcagctcttgcagcgtttccccgaacatgcgaagcgcatttctgatctccggaatggaatggccgcctatgtagagtgcaaggagagttttgatttctttgaaaagctgtacaatcaggcagtgaaagagaagagaacgggtatcgccgccgtctgtgaaaaattcagacagaagcatgaccacgccaccgctcggtgtgagccagtcgtgattgtgctccgcggagacgcggggcaagggaaatctttatcaagtcaggttattgcccaggccgtctccaagaccattttcggccggcaatctgtgtattcccttccccccgattcggatttctttgatggctatgaaaatcagtttgcagcaataatggatgatctagggcaaaatcctgatggctctgatttcactacgttctgtcagatggtttcgactaccaattttctccccaatatggctagtctagagagaaagggcaccccctttacatctcagcttgtggtggcaactaccaatctgcctgagtttagacctgtcacaatagcccattaccctgctgttgagagaaggataactttcgactattcagtgtctgctggtccagtctgctccaaaacagaggccgggtataaggttttggatgttgaaagagcctttaggcctaccggtgaggctcctcttccatgcttccagaataactgccttttccttgagaaagctgggctccagttcagagataaccgaactaaagagatcatttccctggtagatgtgattgagagagccgtggctaggattgaaaggaagaagaaagttctcacaaccgtgcagacccttgtggcacaaggtccagtagacgaggtcagtttccattccgtagtccagcagcttaaagcaagacagcaagcgacagatgaacagcttgaggaattgcaagaggctttcgcgaaagtacaggagcgtaactctgtgttttctgattggttgaagatttctgcaatgttgtgtgctgcgactctggcactttcccaagttgtcaagatggccaaggcggtgaagcagatggtcaagcctgatctggttcgtgtgcaattggatgagcaggagcagggaccttacaatgagacagcgagagttaaaccaaaaacactgcagttgttggacattcagggaccaaaccctgtgatggactttgaaaaatatgtagccaaacatgtaaccgcccccattggttttgtctaccccactggggtgagcacccagacttgcctccttgtgagaggccgcaccttggtagtaaatagacacatggccgagtctgactggacttccatagtagtgcgtggagtcacacacgcccgctctactgttaaaattttggccatagctaaagcaggcaaagagactgacgtatctttcatccgcctctcttctggtcctctattcagagacaatacatccaaatttgtcaaggctggtgatgtacttcctactggtgccgctccagtcacggggataatgaacacggacatacccatgatgtacacaggaaccttcctgaaagctggtgtgtcagtcccagtggaaaccggccagacctttaatcactgtattcattacaaggctaacacacgaaagggctggtgtggatcagccctactggcagatcttggaggaagcaagaaaatccttggcatccattctgctggctctatgggaatagccgccgcctcgattgtgtcacaggagatgattcgggcggtagtgaatgcctttgagccacagggtgctctcgagagattgccagatgggccccgtattcacgtaccacgtaaaacagcactacgccccaccgttgcccgtcaagtcttccaaccagcatatgccccggctgttctatcgaaatttgaccctagaacagaggctgatgtagatgaagtggctttctccaaacatacctccaaccaggaaagcctcccaccagtgtttagaatggtagccaaagagtatgccaatagagttttcaccttgctgggaaaagacaatggccgtctgactgtaaagcaggctttggaaggactggaggggatggaccccatggacaggaacacctccccggggcttccatatactgcgctaggaatgcgcagaacagatgtcgtagattgggaatcagccaccctgatcccgtttgcggcagaaagattaagaaaaatgaatgaaggagacttttccgaagttgtctatcaaacattcctcaaggatgagcttagaccgatagagaaggttcaagccgccaagacacggattgtagatgttccaccatttgagcattgcattctgggtagacaattgttgggaaagtttgcatcaaagttccagacccaaccgggtctggaactaggatcagccattggatgtgacccagatgtacactggactgccttcggtgtcgccatgcaaggttttgagcgtgtctacgatgtggactactccaactttgattcgacccattcggtggcaatgttccgcttattggctgaggaatttttcactccagagaatggttttgaccccctgactagagaatatcttgagtcattagccatttcaacccatgcgtttgaggagaagcgctttctgataaccggtggtctcccatcaggttgtgcagcgacctcaatgctaaacactataatgaataatataataattagggcgggtttgtatctcacgtataaaaattttgaatttgatgatgtgaaggtgttgtcgtacggagatgatctccttgtggccacaaattaccaattggattttgataaggtgagagcaagcctcgcaaagacaggatataagataactcccgctaacacaacttctacctttcctcttaattcgacgcttgaagacgttgtcttcttaaaaagaaagtttaagaaagagggccctctgtatcggcctgtcatgaacagagaggcgttggaagcaatgttgtcatactatcgtccagggactctatctgagaaactcacttcgatcactatgcttgccgttcattctggcaagcaggaatatgatcggctctttgccccattccgtgaggtaggggttgtcgtgccatcattcgagagtgtggagtacagatggaggagtctgttctggtagtagtgtagtcactggcacaacgcgttacccggtaagccaatcgggtatacacggtcgtcatactgcagacagggttcttctactttgcaagatagtctagagtagtaaaataaatagatagag

SEQ ID NO: 3 - DNA, 2856 bp, Bos taurus
gtactggggtgggttgcctttctcttctccaggggatttatctgacccagggattgaacctgagtctcctgcatttgcagctagattctttacggctgagccacctgggaagcccattcgcttctgctgctgctgctgctgctaagttgcttcagtcgtgtccgacctgtgcgacgccatagacagcagcccaccaggtccccgtccctgggattctccaggcaagaacattggagtgggttgccatttcctcctccaatgcatgaaagtgaaaagtgaaagtgaagtcactcagttgtgtccgaccctcagcgaccccatggactgcagccttccagaatggggtgccattgccttctcctcgcttctgctacctcccctttaaaaagaaaacctatggggtgggctctcaagctgagaccctgtgtgcacagccctctggctggtggcagtggagacgggatgatgacaagcctgggggacatgaccccagagaaggaacgggaacaggatgagtgagaggaggttctaaattatccattagcacaggctgccagtggtccttgcataaatgtatagagcacacaggtggggggaaagggagagagagaagaagccagggtataaaaatggcccagcagggaccaattccaggatcccaggacccagttcaccagacgactcagggtcctgtggacagctcaccagctatgatggctgcaggtaagctcgctaaaatcccctccattcgcgtgtcctaaaggggtaatgcggggggccctgccgatggatgtgttcagagctttgggctttagggcttccgaatgtgaacataggtatctacacccagacatttggccaagtttgaaatgttctcagtccctggagggaagggtaggtggggctggcaggagatcaggcgtctagctccctggggccctccgtcgcggccctcctggtctctccctaggcccccggacctccctgctcctggctttcgccctgctctgcctgccctggactcaggtggtgggcgccttcccagccatgtccttgtccggcctgtttgccaacgctgtgctccgggctcagcacctgcatcagctggctgctgacaccttcaaagagtttgtaagctcccgagggatgcgtcctaggggtggggaggcaggaaggggtgaatccacaccccctccacacagtgggaggaaactgaggagttcagccgtattttatccaagtagggatgtggttaggggagcagaaacgggggtgtgtggggtggggagggttccgaataaggcggggaggggaaccgcgcaccagcttagacctgggtgggtgtgttcttcccccaggagcgcacctacatcccggagggacagagatactccatccagaacacccaggttgccttctgcttctctgaaaccatcccggcccccacgggcaagaatgaggcccagcagaaatcagtgagtggcaacctcggaccgaggagcaggggacctccttcatcctaagtaggctgccccagctcccgcaccggcctggggcggccttctccccgaggtggcggaggttgttggatggcagtggaggatgatggtgggcggtggtggcaggaggtcctcgggcagaggccgaccttgcagggctgccccagacccgcggcacccaccgaccacccacctgccagcaggacttggagctgcttcgcatctcactgctcctcatccagtcgtggcttgggcccctgcagttcctcagcagagtcttcaccaacagcttggtgtttggcacctcggaccgtgtctatgagaagctgaaggacctggaggaaggcatcctggccctgatgcgggtggggatggcgttgtgggtcccttccatgtgggggccatgcccgccctctcctggcttagccaggagaatgcacgtgggcttggggagacagatccctgctctctccctctttctagcagtccagccttgacccaggggaaaccttttccccttttgaaacctccttcctcgcccttctccaagcctgtaggggagggtggaaaatggagcgggcaggagggagctgctcctgagggcccttcggcctctctgtctctccctcccttggcaggagctggaagatggcaccccccgggctgggcagatcctcaagcagacctatgacaaatttgacacaaacatgcgcagtgacgacgcgctgctcaagaactacggtctgctctcctgcttccggaaggacctgcataagacggagacgtacctgagggtcatgaagtgccgccgcttcggggaggccagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgag

SEQ ID NO: 4 - DNA, 2657 bp, Homo sapiens
gaattccgggccatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgaccatcccaggaggcctgtgcacacatcccaggcatgtcccagacacatcccacacgtgtccgtgtggccggccagcctggggagtagtggcaacagcccttccgtccacactcccatccaaggagggttcttccttcctgtggggtcactcttgccattgcctggagggggaccagagcatgtgaccagagcatgtgcccagcccctccaccaccaggggcactgccgtcatggcaggggacacagttgtccttgtgtctgaaccatgtcccagcacgggaattctagacatacgtggtctgcggacagggcagcgcccccagcccatgacaagggagtcttgttttctggcttggtttggggacctgcaaatgggaggcctgaggccctcttcaggctttggcagccacagatacttctgaacccttcacagagagcaggcaggggcttcggtgccgcgtgggcagtacgcaggtcccaccgacactcacctgggagcacggcgcctggctcttaccagcgtctggcctagaggaagcctttgagcgacctttgggcaggtttctgcttcttctgttttgcccatggtcaagtccctgttccccaggcaggtttcagctgattggcagcaggctccctgagtgatgagcttgaacctgtggtgtttctgggcagaagcttatcttttttgagagtgtccgaagatgaaggcatggcgatgcccgtcctctggcttgggttaattcttcggtgacactggcattgctgggtggtgatgcccgtcctctggcttgggttaattcttcggtgacactggcgttgctgggtggcaatgcccgtcctctggcttgggttaattcttcggtgacactggcgttgctgggtggcgatgcccgtcctctggcttgggttaattcttggatgacgtcggcgttgctgggagaatgtgccgttcctgccctgcctccacccacctcgggagcagaagcccggcctggacacccctcggcctggacacccctcgaaggagagggcgcttccttgagtaggtgggctccccttgcccttccctccctatcactccatactggggtgggctggaggaggccacaggccagctattgtaaaagcttttt

SEQ ID NO: 5 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 6 - DNA, 1125 bp, Homo sapiens
atggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcgaggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccaccatggactga

SEQ ID NO: 7 - protein, 374 aa, Homo sapiens
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 8 - DNA, 4363 bp, Artificial Sequence
  PAAV-PGK-HMPS3A expression cassette and flanking sequences
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacccggatctatctcgagttgcggccgcatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccaaccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgaatcgattctagaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataattgatcaactagtcctgcaggtttaaacgaattcgcccttcgtgatcaatggctgcgcccgcactagggctggtgtgtggacgttgccctgagctgggtctcgtcctcttgctgctgctgctctcgctgctgtgtggagcggcagggagccaggaggccgggaccggtgcgggcgcggggtcccttgcgggttcttgcggctgcggcacgccccagcggcctggcgcccatggcagttcggcagccgctcaccgatactcgcgggaggctaacgctccgggccccgtacccggagagcggcaactcgcgcactcaaagatggtccccatccctgctggagtatttacaatgggcacagatgatcctcagataaagcaggatggggaagcacctgcgaggagagttactattgatgccttttacatggatgcctatgaagtcagtaatactgaatttgagaagtttgtgaactcaactggctatttgacagaggctgagaagtttggcgactcctttgtctttgaaggcatgttgagtgagcaagtgaagaccaatattcaacaggcagttgcagctgctccctggtggttacctgtgaaaggcgctaactggagacacccagaagggcctgactctactattctgcacaggccggatcatccagttctccatgtgtcctggaatgatgcggttgcctactgcacttgggcagggaagcggctgcccacggaagctgagtgggaatacagctgtcggggaggcctgcataatagacttttcccctggggcaacaaactgcagcccaaaggccagcattatgccaacatttggcagggcgagtttccggtgaccaacactggtgaggatggcttccaaggaactgcgcctgttgatgccttccctcccaatggttatggcttatacaacatagtggggaacgcatgggaatggacttcagactggtggactgttcatcattctgttgaagaaacgcttaacccaaaaggtcccccttctgggaaagaccgagtgaagaaaggtggatcctacatgtgccataggtcttattgttacaggtatcgctgtgctgctcggagccagaacacacctgatagctctgcttcgaatctgggattccgctgtgcagccgaccgcctgcccaccatggactgaactagtagatccagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 4075 bp, Artificial Sequence
  pAAV-SAF302 expression cassette and flanking sequence
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccaaccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 10 - DNA, 130 bp, Artificial Sequence
  ITR1
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 11 - DNA, 141 bp, Artificial Sequence
  ITR2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 12 - DNA, 1721 bp, Artificial Sequence
  CAG
tgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 13 - DNA, 1509 bp, Homo sapiens
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccaaccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga

SEQ ID NO: 14 - DNA, 6672 bp, Artificial Sequence
  Plasmid
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcatcgattgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagcgccaccatgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccaaccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 15 - DNA, 7336 bp, Artificial Sequence
  pAAV-rh10
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaattgcttgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgcc

SEQ ID NO: 16 - DNA, 17867 bp, Artificial Sequence
  pHGTI
tgtggaattgtgagcggataacaatttcacacaggaaacagctatgaccatgattacgccaagctacgtaatacgactcactagtgggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatgtttaaacggatccacgatatcctgcaggaagctttctggcgcctggccggcgcaattgaatgggcccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcttcgcttggtaataaagcccgcttcggcgggcttttttttgttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttg

SEQ ID NO: 17 - DNA, 510 bp, Homo sapiens
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccga

(19) United States
(12) Patent Application Publication
(10) Pub. No.: US 2023/0124994 A1
(43) Pub. Date: Apr. 20, 2023

(54) COMPOSITIONS AND METHODS FOR THE TREATMENT OF SANFILIPPO DISEASE AND OTHER DISORDERS

(71) Applicant: LYSOGENE, Neuilly sur Seine (FR)

(72) Inventors: Karen PIGNET-AIACH, Neuilly sur Seine (FR); Michael HOCQUEMILLER, Neuilly sur Seine (FR); Olivier DANOS, Neuilly sur Seine (FR)

(21) Appl. No.: 17/628,015

(22) PCT Filed: Jul. 17, 2020
(86) PCT No.: PCT/US20/42447

§ 371 Date: Jan. 18, 2022

Related U.S. Application Data
(60) Provisional application No. 62/875,809, filed on Jul. 18, 2019.

(51) Int. Cl. C12N 15/86; C12N 9/14; A61K 45/06; A61K 38/46; A61P 3/00

(52) U.S. Cl. CPC C12N 15/86; C12N 9/14; C12Y 310/01001; A61K 45/06; A61K 38/46; A61P 3/00; C12N 2750/14143; C12N 2750/14171; C12N 2830/50; A61K 48/00

(57) ABSTRACT

The present disclosure provides novel vectors and methods useful in treating genetic diseases, brain disorders, and neurological diseases and disorders, including gene therapy vectors and methods of administering such to a subject in need thereof.

### CROSS REFERENCE TO RELATED APPLICATIONS

This application is a U.S. National Stage Application under 35 U.S.C. § 371 of International Application No. PCT/US2020/042447, filed Jul. 17, 2020, which claims the benefit of priority to U.S. Provisional Application No. 62/875,809, filed on Jul. 18, 2019, each of which is hereby incorporated by reference into this application in its entirety.

### SEQUENCE LISTING

The Sequence Listing associated with this application is provided in text format in lieu of a paper copy, and is hereby incorporated by reference into the specification. The name of the text file containing the Sequence Listing is LYSO-003_01WO_SeqList_ST25.txt. The text file is about 86 KB, was created on Jul. 17, 2019, and is being submitted electronically via EFS-Web.

### BACKGROUND

### Field

In some embodiments, the present invention is directed to gene therapy vectors, as well as methods of using such gene therapy vectors, alone or in combination with one or more immunosuppressants, in the treatment of genetic diseases, including lysosomal storage disorders, and brain diseases and disorders.

### Description of the Related Art

Mucopolysaccharidosis type III (MPSIII), also called Sanfilippo syndrome, is a rare lysosomal storage disease (LSD) belonging to the group of mucopolysaccharidosis (MPS). These lysosomal storage diseases (LSD) are caused by a missing or dysfunctional digestive protein, leading to the subsequent accumulation of substrates in the cell, resulting in very severe cellular and organ dysfunctions. Mucopolysaccharides or glycosaminoglycans (GAGs) are constantly recycled macromolecules of essential importance for normal cell function. In MPS, a deficiency in one of the lysosomal enzyme that participates in the stepwise degradation of GAGs leads to their accumulation, and results in severe cell dysfunction. MPSs form a complex group of genetic diseases differing by their genetic origin, biochemical and physiological disturbances and clinical manifestations. Depending of the mutated gene, the catabolism of one or several types of GAGs will be blocked, some enzymes being involved in the degradation pathway of multiple GAG species.

Mucopolysaccharidosis type III (MPSIII) is a rare lysosomal storage disease which affects between 0.7 and 1.8 per 100,000 live birth (0.73 in France and 1.7 in UK) and in which an autosomal recessive genetic defect results in the accumulation of partially degraded oligosaccharides of heparan sulfate.

Sanfilippo type A syndrome or Mucopolysaccharidosis type III type A (MPSIIIA) is caused by a autosomal recessive genetic defect of N-sulfoglycosamine sulfohydrolase (SGSH). This enzyme is ubiquitously expressed in tissues and is involved in the step-wise degradation of heparan sulfate (HS). In its absence, partially degraded oligosaccharides accumulate at toxic levels.

Sanfilippo type A syndrome is a severe debilitating and life threatening lysosomal storage disease affecting children. The clinical manifestations are mainly neurological with early symptoms usually observed during the first 5 years of life, leading to a progressive deterioration of cognitive abilities. Affected children require specific care in the early childhood and progressively develop profound mental retardation with minimal somatic manifestations [1]. Death occurs usually before the age of 15 although some patients are still alive after the age of 20. There is currently no treatment available for patients with Sanfilippo type A syndrome.

The rationale for therapeutic approaches in MPS is based on the observation that delivery of the missing enzyme reverses phenotypic abnormalities in genetically deficient cells. MPS enzyme substitution treatment relies on the internalization of extracellular enzyme by deficient cells, through binding to the mannose-6-phosphase receptors. Enzyme replacement therapy (ERT) is being explored, but is not currently available for MPSIIIA.

MPSs have been recognized as prime candidate diseases for gene therapy. Using gene transfer ex vivo or in situ, the missing enzyme may be produced and distributed to the organism by a group of genetically modified cells. Numerous studies in animal models of MPS have described the effects of genetic modification of tissues including bone marrow, skin, muscle, liver and brain, resulting in the production of a therapeutically active enzyme. However, when vectors are administered in the periphery, the produced enzyme does not cross the blood-brain barrier. Only very high doses of enzyme in the circulation may result in detectable transport into the brain. This drawback to gene therapy methods is not limited to MPS, but also limits the use of gene therapy to treat other brain diseases and disorders.

Accordingly, there is clearly a need in the art for gene therapy vectors and methods that provide direct administration into the brain for treatment of MPSs and other diseases and disorders affecting the brain. The present invention addresses these needs by providing improved gene therapy vectors, as well as other improved methods applicable to the treatment of genetic diseases and neurological and brain diseases and disorders generally, and MPSs more specifically, including Sanfilippo type A.

### BRIEF SUMMARY

The present disclosure provides novel vectors and methods useful in treating genetic diseases, brain disorders, and neurological diseases and disorders, including gene therapy vectors.

In one embodiment, the present disclosure includes a replication deficient adeno-associated virus (AAV)-derived vector comprising an expression cassette comprising in the following 5′ to 3′order: a promoter sequence; a polynucleotide sequence encoding a human N-sulfoglucosamine sulfohydrolase (SGSH) polypeptide or an active variant thereof; and a polyadenylation (polyA) sequence. In some embodiments, the vector does not include a polynucleotide sequence encoding a human sulfatase-modifying factor 1 (SUMF1) polypeptide, or any active variant thereof. In certain embodiments, the promoter sequence is operably linked to the polynucleotide sequence encoding a human N-sulfoglucosamine sulfohydrolase polypeptide, or an active variant thereof. In some embodiments, polynucleotide sequence encoding a human N-sulfoglucosamine sulfohydrolase comprises a sequence according to SEQ ID NO: 13.

In some embodiments, the promoter sequence is a CMV early enhancer/chicken β actin (CAG) promoter. In some embodiments, the CAG promoter comprises a sequence according to SEQ ID NO: 12. In some embodiments, the vector does not include an IRES sequence. In some embodiments, the polyA sequence is derived from a human growth hormone 1 polyA sequence. In some embodiments, the poly A sequence comprises a sequence according to SEQ ID NO: 17. In particular embodiments, the vector is AAV serotype rh10. In some embodiments, the vector comprises a sequence according to SEQ ID NO: 9. In some embodiments, SEQ ID NO: 9 comprises the sequence of DNA encapsidated in LYS-SAF302 particles.

In certain embodiments of the vector, the expression cassette comprises or consists of, in the following 5′ to 3′order: a promoter sequence derived from a CAG promoter sequence; a polynucleotide sequence encoding a human N-sulfoglucosamine sulfohydrolase (SGSH) polypeptide or an active variant thereof; and a polyA sequence derived from a human growth hormone polyA sequence. In particular embodiments of the vectors, the expression cassette is flanked by two AAV2 inverted terminal repeat (ITR) sequences, wherein one of the two AAV2 ITR sequences is located 5′ of the expression cassette and one of the two AAV2 ITR sequences is located 3′ of the expression cassette. In some embodiments, the two ITR termini are the only cis-acting elements required for genome replication and packaging. In some embodiments, each ITR contains about 100, about 110, about 120, about 130, about 140, about 145, about 150, or about 160 nucleotides In some embodiments, the ITR sequence located at the 5′ end of the expression cassette comprises the nucleotide sequence according to SEQ ID NO: 10. In some embodiments, the ITR sequence located at the 3′ end of the expression cassette comprises the nucleotide sequence according to SEQ ID NO: 11.

In particular embodiments, the vector further comprises an AAVrh.10 capsid or serotype.

In some embodiments, the DNA of the vector comprises or consists of 4.07 kb and the sequence molecular weight is 1257.4 kDa. In some embodiments, the SGSH sequence comprises or consists of 1.35 kb and the molecular weight of the SGSH DNA is 471.3 kDa.

Related embodiments include any of the expression cassettes described herein and plasmids comprising an expression cassette described herein. Further related embodiments include host cells comprising a plasmid or a vector described herein. In particular embodiments, the host cell is ex vivo. In one embodiment, the host cell is a 293 cell, such as a 293T cell.

In another embodiment, the present disclosure includes a method or process for producing a vector according to the present disclosure, comprising introducing a plasmid comprising an expression cassette described herein, wherein the expression cassette is flanked by two AAV2 internal terminal repeat (ITR) sequences, into a host cell, and culturing the host cell to produce a vector described herein. In particular embodiments, the expression cassette comprises or consists of, in the following 5′ to 3′order: a promoter sequence; a polynucleotide sequence encoding a human N-sulfoglucosamine sulfohydrolase (SGSH) polypeptide or an active variant thereof; and a polyadenylation (polyA) sequence. In certain embodiments, the method or process further comprises introducing a helper plasmid into said host cell. In particular embodiments, said helper plasmid comprises polynucleotide sequences encoding: capsid proteins, e.g., from AAVrh.10; replication genes, e.g., from AAV2; and helper functions, e.g., derived from adenovirus serotype 5. In some embodiments, the present disclosure includes a method or process for producing a vector according to the present disclosure, comprising the use of three plasmids. In some embodiments, a first plasmid comprises an expression cassette comprising or consisting of, in the following 5′ to 3′ order: a promoter sequence, a polynucleotide sequence encoding a human SGSH polypeptide or an active variant thereof, and a polyadenylation sequence. In some embodiments, the expression cassette is flanked by two AAV ITR sequences. In some embodiments, the expression cassette and flanking sequences together comprise or consist of a sequence according to SEQ ID NO: 9. In some embodiments, the first plasmid comprises or consists of a polynucleotide according to SEQ ID NO: 14. In some embodiments, a second plasmid comprises a polynucleotide sequence encoding a capsid protein, for example, capsid protein from AAVrh10. In some embodiments, the second plasmid comprises a polynucleotide according to SEQ ID NO: 15. In some embodiments, a third plasmid provides helper functions, for example, helper functions derived from an adenovirus. In some embodiments, the third plasmid comprises a polynucleotide according to SEQ ID NO: 16. In some embodiments, the methods and processes provided herein comprise introducing into a host cell the first, second, and third plasmids. For example, in some embodiments, the methods and processes provided herein comprise introducing into a host cell plasmids comprising sequences according to SEQ ID NOs: 14, 15, and 16. In some embodiments, the plasmids are introduced into the host cell and the host cell is cultured to produce the vector described herein. In some embodiments, the resulting vector is termed SAF302 (also referred to herein as LYS-SAF302).

Further related embodiments include a composition comprising a vector described herein and a pharmaceutically acceptable carrier. In some embodiments, the present disclosure provides formulations comprising the vector provided herein and PBS buffer with no excipients or preservatives. In some embodiments, the PBS buffer comprises KCl, KH₂PO₄, and NaCl, Na₂HPO₄. In further embodiments, the PBS buffer comprises about 2.67 mM KCl, about 1.47 mM KH₂PO₄, about 137.9 mM NaCl, and about 8.05 mM Na₂HPO₄. In some embodiments, the formulation has a pH of about 6.8 to about 7.8, or from about 7.0 to about 7.6, or from about 7.2 to about 7.4. In some embodiments, the formulation is suitable for storage at −80° C.±10° C. In some embodiments, the formulations provided herein are filtered through a 0.2 micron inline filter prior to administration to a subject.

Another related embodiment of the present disclosure includes a method of treating Sanfilippo type A syndrome, comprising administering a composition comprising a vector described herein to a subject in need thereof. In particular embodiments, the vector comprises an expression cassette comprising or consisting of, in the following 5′ to 3′order: an AAV2 ITR sequence; a CAG promoter sequence; a polynucleotide sequence encoding a human N-sulfoglucosamine sulfohydrolase (SGSH) polypeptide or an active variant thereof; and a human growth hormone polyA sequence; wherein the CAG promoter sequence is operably linked to the polynucleotide sequence encoding the SGSH polypeptide. In certain embodiments, the expression cassette does not include a polynucleotide sequence encoding a SUMF1 polypeptide, or any active variant thereof. In some embodiments, the expression cassette is flanked by two AAV2 internal terminal repeat (ITR) sequences.

In particular embodiments of this method, the composition is administered via intracerebral injection to one or more sites within the subject's brain. In certain embodiments, the composition is administered to two to twenty sites, four to sixteen sites, eight to sixteen sites, or six to twelve sites in the subject's brain. For example, in some embodiments, the composition is administered to the subject's brain via 2-4 injections per hemisphere, for a total of 4-8 injections per subject. In one embodiment, the composition is administered to six sites within the subject's brain. In some embodiments, the composition is administered to three sites per hemisphere. In certain embodiments, the composition is administered via injection through one or more burr holes in the subject's head, e.g., six burr holes in the subject's head, wherein the composition is administered through each burr hole or track at a single depth. Each injection site may be deep or superficial. For example, a deep injection is performed at a depth of about 1.5 cm to about 3.0 cm, or about 1.7 cm to about 2.5 cm, or about 2 cm from the cortical surface; and a superficial injection is performed at a depth of about 0.5 cm to about 2.0 cm, or about 0.7 cm to about 1.5 cm, or about 1 cm from the cortical surface. In certain embodiments, the sites are selected from one or more of: anterior right superficial, anterior right deep, anterior left superficial, anterior left deep, medial right superficial, medial right deep, medial left superficial, medial left deep, posterior right superficial, posterior right deep, posterior left superficial, and posterior left deep.

In certain embodiments, a total of about 1.0×10¹⁰ gc to about 1.0×10¹⁴ gc, about 5.0×10¹⁰ gc to about 5.0×10¹³ gc, about 5.0×10¹⁰ gc to about 1.0×10¹³ gc, about 1.0×10¹¹ gc to about 1.0×10¹³ gc, about 1.0×10¹¹ gc to about 5.0×10¹² gc, about 5.0×10¹¹ gc to about 5.0×10¹² gc, or about 5×10¹⁰ to about 5×10¹³ gc, or about 7×10¹⁰ to about 7×10¹³ gc of viral vector is administered to the subject. In certain embodiments, a total of about 7.2×10¹² gc of viral vector is administered to the subject. In certain embodiments, about 0.8×10⁹ gc to about 0.8×10¹³ gc, about 0.4×10¹⁰ gc to about 0.4×10¹³ gc, about 0.4×10¹⁰ gc to about 0.8×10¹² gc, about 0.8×10¹⁰ gc to about 0.8×10¹² gc, about 0.8×10¹⁰ gc to about 0.4×10¹² gc, or about 0.4×10¹¹ gc to about 0.4×10¹² gc of viral vector is administered to each site of the subject. In particular embodiments, about 1.2×10¹² gc of viral vector is administered to each site of the subject. In particular embodiments, about 1.2×10¹² gc of viral vector is administered to each of six sites in the subject's brain or white matter, such that about 7.2×10¹² gc of viral vector is administered to the subject. In certain embodiments, the volume of composition comprising the gene therapy vector that is administered to each site is about 10 μl to about 600 μl. In some embodiments, the volume of composition comprising the gene therapy vector that is administered to each site is about 500 μl. In particular embodiments, the infusion rate for administration of the composition comprising the gene therapy vector is about 0.1 μl/min to about 10 μl/min, about 0.2 μl/min to about 8 μl/min, about 0.3 μl/min to about 6 μl/min, or about 0.4 μl/min to about 4.0 μl/min, or about 0.4 μl/min to about 3.0 μl/min, or about 5.0 μl/min. In some embodiments, the method further comprises administering an immunosuppressive regimen to the subject. In some embodiments, the immunosuppressive regimen comprises tacrolimus, mycophenolate mofetil, and prednisone.

In a related embodiment, the present disclosure includes a vector described herein for use as a medicament in the treatment Sanfilippo type A syndrome. In particular embodiments, the vector for use as a medicament in the treatment Sanfilippo type A syndrome comprises the following expression cassette in 5′ to 3′ order, wherein the CAG promoter sequence is operably linked to the polynucleotide sequence encoding SGSH: an AAV2 ITR sequence; a CAG promoter sequence; a polynucleotide sequence encoding a human N-sulfoglucosamine sulfohydrolase (SGSH) polypeptide or an active variant thereof; a human growth hormone polyA sequence; and an AAV2 ITR sequence. The vector for use as a medicament is for administration via the methods provided herein.

In particular embodiments of the vector for use as a medicament in the treatment Sanfilippo type A syndrome, the medicament further comprises a pharmaceutically acceptable support, carrier, diluent, or excipient. In certain embodiments, the medicament is an emulsion or an aqueous solution. In certain embodiments, the medicament comprises a buffer with no excipients or preservatives. In further embodiments, the buffer is a PBS buffer.

In a related embodiment, the present disclosure includes a process for producing a vector of the present disclosure, comprising introducing a plasmid comprising an expression cassette of the present disclosure into a host cell; and culturing the host cell to produce the vector. In particular embodiments, the plasmid further comprises AAV2 ITRs flanking the expression cassette.

In a further related embodiment, the present disclosure includes a plasmid comprising an expression cassette of the present disclosure. In particular embodiments, the plasmid further comprises AAV2 ITRs flanking the expression cassette.

In another related embodiment, the present disclosure includes a host cell comprising a vector, plasmid, or expression cassette of the present disclosure. In particular embodiments, the host cell is a human embryonic kidney cell, e.g., a 293 or 293T cell.

In some embodiments, the present disclosure provides a kit comprising (a) vector that comprises a plasmid provided herein and (b) instructions for use thereof. In some embodiments, the kit comprises a vector comprising a plasmid, wherein the plasmid comprises an expression cassette as provided herein and further comprises AAV2 ITRs.

In addition, any of the methods and uses described herein may be used to increase expression of a SGSH polypeptide or variant thereof in a subject in need thereof. In some embodiments, the methods and uses described herein increase and/or restore SGSH activity in the brain of a subject in need thereof. In some embodiments, the methods and uses restore at least about 10%, at least about 15%, at least about 20%, at least about 25%, at least about 30%, or more of normal SGSH activity in the brain of the subject.

### BRIEF DESCRIPTION OF THE DRAWINGS

FIG. 1 is a schematic representation of an Adeno Associated Virus vector construct, the first generation LYS-SAF301 gene therapy vector. In this figure AAV2 ITR refers to AAV2 internal terminal repeat, muPGK refers to murine phosphoglycerol kinase promoter, SGSH refers to human N-sulfoglucosamine sulfohydrolase, EMCV ires refers to encephalomyocarditis virus ires, SUMF1 refers to human sulfatase-modifying factor 1, and BGHpA refers to bovine growth factor polyA.

FIG. 2A is a schematic representation of pAVV-PGK-HMPS3A plasmid used for production of the first generation LYS-SAF301 gene therapy vector. In this figure, ITR refers internal terminal repeat, PGK refers to phosphoglycerol kinase promoter, SGSH refers to human N-sulfoglucosamine sulfohydrolase, SUMF1 refers to human sulfatase-modifying factor 1, IRES refers to an internal ribosomal entry site, and KanR refers to kanamycin resistance.

FIG. 2B is a schematic representation of the pPAK-MARH10 helper plasmid. In this figure, AAV2 rep gene refers to genes for replication of AAV2, AAVrh10 cap genes refers to genes coding for AAV rhesus 10 capsid, MMTV promoter refers to mouse mammary tumor virus promoter, KanR to kanamycin resistance, and Ad5 E4 genes refers to adenovirus 5 E4 genes.

FIG. 3 is a schematic representation of the LYS-SAF302 expression cassette and flanking sequences.

FIGS. 4A and 4B represent the sequence of DNA encapsidated in LYS-SAF302 particles (SEQ ID NO: 9). FIGS. 4C-4D represent the full sequence of the plasmid p-LYS-SAF-T5 (SEQ ID NO: 14). In FIGS. 4C and 4D, the ITRs, CAG promoter, cDNA for SGSh, poly A site, and AmpR gene are indicated. FIGS. 4E-4F represent the sequence of the plasmid containing the capsid rh10 (pAAV2-rh10; SEQ ID NO: 15) sequence. In FIGS. 4E and 4F, the AAV2 REP, CAP1, Lac O, ColE1 origin, AmpR, F1 origin, and Lac a components of the plasmid are indicated. FIGS. 4G-4K represent the sequence of the helper plasmid pHGTI, i.e., plasmid with helper functions of adenovirus (SEQ ID NO: 16). In FIGS. 4G-4K, the VA, Rads, E4, L5, E2A, AmpR, and ColE1 origin components of the plasmid are indicated.

FIG. 5 shows the effects of LYS-SAF301 and LYS-SAF302 on SGSH activity across all brain regions of MPSIIIA mice at 4 weeks post injection. Data are mean % SGSH activity relative to WT levels SEM; ****p<0.0001, **p<0.01 and ns=non-significant giving significance vs WT unless indicated with overhead bars.

FIG. 6 shows the effect of LYS-SAF301 and LYS-SAF302 on the amount of total HS across all brain regions of MPS IIIA mice at 4 weeks post injection. Average relative amounts of HS in all brain regions (average of regions 1-5) following peak area quantification from RP-HPLC of AMAC-labelled disaccharides. Data are mean±SEM; *p<0.05 significance vs WT.

FIG. 7A-E shows the effect of LYS-SAF301 and LYS-SAF302 on amounts of inflammatory cytokines across all brain regions of MPS IIIA mice at 4 weeks post injection. Protein levels for MIP-1α (7A), MCP-1 (7B), IL-1α (7C), RANTES (7D) and KC (7E) as measured by CBA flex sets for all brain regions. Each data point represents the value for each individual mouse across all 5 brain regions. P values are from two-way ANOVA with Tukey's multiple comparisons test, *=P<0.05, **=P<0.01, ***=P<0.001, ****=P<0.0001.

FIG. 8 shows the SGSH expression from LYS-SAF302 at the indicated MOI.

FIG. 9 shows the enzymatic activity of SGSH produced from LYS-SAF302 at the indicated MOI.

FIG. 10 shows the intraparenchymal injection strategy of LYS-SAF302 in MPSIIIA mice. The injection sites (white arrows) are shown from a lateral perspective with location of the hemicoronal brain slices. MPSIIIA mice received stereotactic injection of LYS-SAF302 at 5 weeks of age using a Hamilton syringe (n=10/gender/group). Vectors were administered at a dose of 8.6E+08 vg (low dose), 4.1E+10 vg (medium dose) or 9.0E+10 vg (high dose) in 8 μl delivered at 0.2 μl/min via 2 μl into each of the left and right striatum and 2 μL into each of the left and right thalamus. Location of the five hemi-coronal slices are presented.

FIG. 11 shows the dose dependent effects of LYS-SAF302 on SGSH activity in brain slices of MPS IIIA mice at 12 and 25 weeks post injection. (□ male; • female). P values are from one-way ANOVA with post-hoc Bonferroni testing, *=P<0.05, **=P<0.01, ***=P<0.001, ****=P<0.0001. Mouse #225 (17-week old male MPS IIIA low dose group) was an outlier in each assay, exhibiting no increase in SGSH activity (c.f. MPS IIIA vehicle mice). In the 30-week cohort, male low dose-treated MPS IIIA mice #422, #383 and #448 were outliers, exhibiting no increase in SGSH activity (c.f. MPS IIIA vehicle mice).

FIG. 12 shows the dose dependent effects of LYS-SAF302 on amounts of HS in brain slices of MPS IIIA mice at 12 and 25 weeks post injection. (□ male; • female). P values are from one-way ANOVA with post-hoc Bonferroni testing, *=P<0.05, **=P<0.01, ***=P<0.001, ****=P<0.0001. Mouse #225 (17-week old male MPS IIIA low dose group) was an outlier in each assay, exhibiting no reduction in HS (c.f. MPS IIIA vehicle mice). In the 30-week cohort, male low dose-treated MPS IIIA mice #422, #383 and #448 were outliers, exhibiting no reduction in HS (c.f. MPS IIIA vehicle mice). This is in keeping with the low amount of SGSH activity recorded for these mouse samples.

FIG. 13 shows neuropathology analyses of MPS IIIA mice 25 weeks post treatment. FIGS. 13A and 13B show secondary lysosomal storage products GM2 (A) and GM3 (B) gangliosides quantification in brain slice 3 according to the map given in FIG. 10. FIG. 13C shows endo/lysosomal system expansion assessed by LIMP2 staining in inferior collicus. FIG. 13D shows the number of axonal spheroids, assessed by ubiquitin staining in inferior collicus. FIG. 13E shows the extent of astrogliosis, assessed by GFAP staining in inferior collicus. FIG. 13F shows the number of reactive amoeboid-shaped microglia, assessed by isolectin B4 staining, in dentate gyrus. ****p<0.0001 and *p<0.05 calculated from one-way ANOVA with Bonferroni's multiple comparisons test.

FIG. 14A-F shows gadolinium diffusion in dog brain. The figure represents a dog that received four injection of 500 μl (two per hemisphere) of LYS-SAF302 with the MRI contrast agent gadolinium (5 mmol) into the white matter at 10 μL/min (total dose 2.0E+12 vg). FIG. 14A is the left lateral view of a dog brain with the position of coronal sections that include sites of injection. FIGS. 14B-C are MRI images of coronal sections before injection with planned site of injection represented with red dot spots. FIGS. 14D-E are MRI images of coronal sections after injection with gadolinium signal visible into the white matter. FIG. 14F is an anterior view of 3D reconstruction of MRI images with gadolinium signal visible in both hemisphere along the rostro-caudal axis of the white matter. Scale bars=10 mm.

FIG. 15A-C shows SGSH activity distribution in the brain of two NHP that received four injection of 50 μl (two per hemisphere) of LYS-SAF302 into the white matter at 5 μL/min (total dose 7.2E+11 vg). FIG. 15A shows the left lateral view of a NHP brain with location of the hemicoronal brain slices. Rostral injections were between slice 4 to 6 and caudal injection between slice 8 to 10. Greater than 20% increase of SGSH activity relative to vehicle injected controls was observed 6 weeks after injection in 99% of the brain punches analysed for one NHP (FIG. 15B) and 94% of the brain punches analysed for another NHP (FIG. 15C).

FIG. 16A-16E shows the expression of SAF302GFP in mice brain at 4 months post injection. FIG. 16A is a diagram of the intrastriatal injection site relative to the 5 different brain regions. MPSIIIA mice received a stereotactic injection of SAF302GFP at 8-14 weeks of age using a Hamilton syringe (n=6). The vector was administered at a dose of 6.1×10⁹ genome particles in 3 μL delivered via bilateral injection at a depth of 3 mm, 2 mm lateral to the midline in both hemispheres. Animals were sacrificed 4 months after injection, and brains were taken for histological analyses. Coronal brain sections from the bregma+1.7, +0.26, −1.18, and −2.62 mm (FIG. 16B) and a sagittal section at 1.2 mm lateral to the midline (FIG. 16C) were co-labeled with NeuN (red), GFP (green), and DAPI (blue). Scale bars=1,000 μm. (16D and 16E) High-magnification images of the regions indicated by the dashed boxes in (FIG. 16C) show transduction of cells within the hippocampus (FIG. 16D) and the striatum (FIG. 16E). Scale bar=50 μm.

FIG. 17A-E shows transduction of GFAP-positive astrocytes near the injection site following treatment with SAF302GFP. MPSIIIA mice were injected with SAF302GFP at two depths and harvested at 4 weeks (FIG. 17A) or at one depth and harvested at 4 months (FIG. 17B) post injection. Coronal sections at +0.26 relative to the bregma were stained with GFAP (red), GFP (green), and DAPI (blue). Scale bars=1,000 μm. High-magnification images of the cingulum/cortex (1), external capsule/primary somatosensory cortex/striatum boundary (2), and striatum/injection site (3) are shown. Scale bar=20 μm. Quantification of percentage of GFP-positive cells at the indicated bregma as a proportion of total brain area (FIG. 17C-17E). *p<0.05 calculated using a Student's t-test. Representative images are shown, with five mice injected in both hemispheres and quantified per group.

### DETAILED DESCRIPTION

In various embodiments, the present disclosure provides novel compositions and methods useful in treating a variety of diseases and disorders, including, but not limited to, genetic diseases (including those resulting from a gene deletion or mutation leading to reduced expression or lack of expression of an encoded gene product, the expression of an altered form of a gene product, or disruption of a regulatory element controlling the expression of a gene product), neurological diseases and disorders, and diseases and disorders of the brain. As will be appreciated by one of skill in the art, while certain compositions and methods are specifically exemplified herein, the present disclosure is not so limited but includes additional embodiments and uses, including, but not limited to, those specifically described herein. In addition, in the following description, certain specific details are set forth in order to provide a thorough understanding of various embodiments of the disclosure. However, one skilled in the art will understand that the disclosure may be practiced without these details.

### Definitions

Unless defined otherwise, all technical and scientific terms used herein have the same meaning as commonly understood by those of ordinary skill in the art to which the invention belongs. For the purposes of the present disclosure, the following terms are defined below.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

The term “active variant” indicates and encompasses both “biologically active fragments” and “biologically active variants.” Representative biologically active fragments and biologically active variants generally participate in an interaction, e.g., an intra-molecular or an inter-molecular interaction. An inter-molecular interaction can be a specific binding interaction or an enzymatic interaction. Examples of enzymatic interactions or activities include, without limitation, dehydroxylation and other enzymatic activities described herein.

The term “biologically active fragment”, as applied to fragments of a reference polynucleotide or polypeptide sequence, refers to a fragment that has at least about 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of at least one activity (e.g., an enzymatic activity) of a reference sequence. The term “reference sequence” refers generally to a nucleic acid coding sequence or amino acid sequence to which another sequence is being compared. All sequences provided in the Sequence Listing are also included as reference sequences. Included within the scope of the present disclosure are biologically active fragments of at least about 10, 11, 12, 13, 14, 15, 16, 17, 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between.

The term “biologically active variant”, as applied to variants of a reference polynucleotide or polypeptide sequence, refers to a variant that has at least about 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of an activity (e.g., an enzymatic activity) of a reference sequence. Included within the scope of the present disclosure are biologically active variants having at least about 50%, at least about 60%, at least about 70%, at least about 80% at least about 90%, at least about 95%, at least about 98%, or at least about 99% identity with a reference sequence, including all integers in between.

By “coding sequence” is meant any polynucleotide sequence that contributes to the code for the polypeptide product of a gene. By contrast, the term “non-coding sequence” refers to any polynucleotide sequence that does not contribute to the code for the polypeptide product of a gene.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the terms “function” and “functional”, and the like, refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

The recitations “mutation” or deletion,” in relation to a gene refer generally to those changes or alterations in a gene that result in decreased or no expression of the encoded gene product or that render the product of the gene non-functional or having reduced function as compared to the wild-type gene product. Examples of such changes include nucleotide substitutions, deletions, or additions to the coding or regulatory sequences of a target gene, in whole or in part, which disrupt, eliminate, down-regulate, or significantly reduce the expression of the polypeptide encoded by that gene, whether at the level of transcription or translation, and/or which produce a relatively inactive (e.g., mutated or truncated) or unstable polypeptide. In certain aspects, a targeted gene may be rendered “non-functional” by changes or mutations at the nucleotide level that alter the amino acid sequence of the encoded polypeptide, such that the modified polypeptide is expressed, but has reduced function or activity with respect to one or more enzymatic activity, whether by modifying that polypeptide's active site, its cellular localization, its stability, or other functional features apparent to a person skilled in the art.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a polynucleotide or polypeptide is isolated from, or derived from, a particular source, such as a desired organism or a specific tissue within a desired organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition. In the absence of that condition, inducible promoters typically do not allow significant or measurable levels of transcriptional activity. Numerous standard inducible promoters will be known to one of skill in the art.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes both single and double stranded forms of DNA and RNA.

The term “polynucleotide variant” refers to polynucleotides displaying substantial sequence identity with a reference polynucleotide sequence or polynucleotides that hybridize with a reference sequence under stringent conditions that are defined hereinafter. This term also encompass polynucleotides that are distinguished from a reference polynucleotide by the addition, deletion or substitution of at least one nucleotide. Accordingly, the term “polynucleotide variant” includes polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

With regard to polynucleotides and polypeptides, the term “exogenous” refers to a polynucleotide or polypeptide sequence that does not naturally occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides and polypeptides, the term “endogenous” or “native” refers to naturally-occurring polynucleotide or polypeptide sequences that may be found in a given wild-type cell or organism.

An “introduced” polynucleotide sequence refers to a polynucleotide sequence that is added or introduced into a cell or organism. The “introduced” polynucleotide sequence may be a polynucleotide sequence that is exogenous to the cell or organism, or it may be a polynucleotide sequence that is already present in the cell or organism. For example, a polynucleotide can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation “polypeptide variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues. Included are polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing). In particular embodiments, the polypeptide variant maintains at least one biological activity of the reference polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Transformation” refers to the permanent, heritable alteration in a cell resulting from the uptake and incorporation of foreign DNA into the host-cell genome or maintained extrachromosomally within the host cell; also, the transfer of an exogenous gene from one organism into the genome of another organism.

“As used herein, the terms “treatment,” “treat,” “treated” or “treating” refer to prophylaxis and/or therapy, particularly wherein the object is to prevent or slow down (lessen) an undesired physiological change or disorder, such as the development and/or progression of a brain disorder resulting from a mutated gene, such as, e.g., a lysosomal storage disease (LSDs). Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival and/or increased quality of life as compared to expected survival and/or quality of life if not receiving treatment. Those in need of treatment include those already with the condition or disorder (e.g., brain disorder resulting from a mutated gene, such as an LSD) as well as those prone to have the condition or disorder or those in which the condition or disorder is to be prevented. Thus, “treatment” also includes administration of the compounds of the disclosure to those individuals thought to be predisposed to the disease due to familial history, genetic or chromosomal abnormalities, and/or due to the presence of one or more biological markers for the disease, e.g., to inhibit, prevent, or delay onset of the disease, or reduce the likelihood of occurrence of the disease. In particular embodiments, treatment may include any of the following: decrease of developmentally regression, decrease of language impairment or improvement of language development, decrease of motor skill impairment, decrease of intellectual development impairment, decrease of hyperactivity (excess motor activity), improvement in sleep, attention, decrease of physical and mental ability impairment (patients lose complete motor abilities (walking, speech, feeding, etc.), cognitive abilities, severe seizures, decrease of impairment, such as airway obstruction and cardiac failure, or decrease of accumulation of partially degraded heparan sulfate. In certain embodiments, “treatment” includes making the cells able to produce the missing enzyme treating and/or reversing the consequences of the disease, e.g., restoring or providing the function of SGSH gene to a subject, or breaking down the accumulated heparan sulfate.

A “subject” includes a mammal, e.g., a human, including a mammal in need of treatment for a disease or disorder, such as a mammal having been diagnosed with having a disease or disorder or determined to be at risk of developing a disease or disorder. In particular examples, a subject is a mammal diagnosed with a genetic disease, a brain disorder, or a neurological disease or disorder, such as a lysosomal storage disorder, including an MPS, such as MPSIIIA.

By “vector” is meant a polynucleotide molecule, e.g., a DNA molecule derived, for example, from a plasmid, bacteriophage, yeast or virus, into which a polynucleotide can be inserted or cloned. A vector typically contains one or more unique restriction sites and can be capable of autonomous replication in a defined host cell, or be integrable with the genome of the defined host such that the cloned sequence is reproducible. Accordingly, a vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. A vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. “Vectors” also include viruses and viral particles into which a polynucleotide can be inserted or cloned. Such may be referred to as “viral vectors.” “Gene therapy vectors” are vectors, including viral vectors, used to deliver a therapeutic polynucleotide or polypeptide sequence to a subject in need thereof, which is typically a polynucleotide or polypeptide sequence missing, mutated or having deregulated expression in the subject, e.g., due to a genetic mutation in the subject.

A common means to insert a DNA sequence of interest into a DNA vector involves the use of enzymes called restriction enzymes that cleave DNA at specific sites called restriction sites. A “cassette” or “gene cassette” or “expression cassette” refers to a polynucleotide sequence that encodes for one or more expression products, and contains the necessary cis-acting elements for expression of these products, that can be inserted into a vector at defined restriction sites.

The term “wild-type”, as used herein, refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

Gene Therapy Vectors

In certain embodiments, the present disclosure includes gene therapy vectors for the treatment of MPSIIIA. Such gene therapy vectors may be used to deliver a human SGSH polypeptide or active variant thereof to a cell within a subject in need thereof. As described in the accompanying examples, studies have established that the gene therapy vectors of the present disclosure are both efficacious and safe for the treatment of MPSIIIA.

Without wishing to be bound by theory, it is understood that upon administration into the brain parenchyma, the gene therapy vector particles and the enzymes will diffuse locally, as well as be transported along axons to remote anatomical brain structures to allow for the correction of extended brain regions. Upon entry into cells, the gene therapy vector encoding the SGSH polypeptide will be transported into the nucleus where it will undergo a series of molecular transformations resulting in the stable establishment as a double stranded deoxyribonucleic acid (DNA) molecule. This DNA will be transcribed into messenger ribonucleic acids (mRNAs), which in turn will translate into SGSH, the missing enzyme. Transduced cells will express and deliver the SGSH enzyme continuously, thus constituting an intracerebral permanent source of enzyme production to complement the lacking endogenous enzyme. The gene therapy vector described herein is LYS-SAF302, also referred to herein as SAF302 or AAVrh10-SGSH. As described in the accompanying examples, SAF302 is a second generation product that built upon the information gained testing a first-generation product called LYS-SAF301 or SAF301. LYS-SAF301 consisted of a replication deficient adeno-associated virus serotype rh.10 (AAVrh.10) comprised of a defective AAV2 genome containing the SGSH and SUMF1 genes driven by a PGK promotor packaged in a capsid of AAVrh.10. It is also referred to as AAVrh.10-hMPS3A. This first-generation vector was used in clinical studies described herein in children with MPS IIIA disease and was delivered at a dose of 7.2E+11 viral genomes (vg)/patient into the brain white matter, in 12 pre-planned simultaneous frameless stereotaxic injections (60 μL/injection) at two injection depths. The dose and the surgical intervention were well tolerated and no related adverse events were reported.

The present disclosure provides an improved, second generation product, LYS-SAF302, which is 2.7-fold more potent compared to the first generation product in terms of expression from the transduced cells. In addition, the present disclosure provides an improved delivery system that allows injection of higher doses and higher volumes at increased flow rates without reflux. The delivery systems and methods provided herein result in broad brain distribution and enhanced efficacy. As described in the accompanying Examples, efficacy studies of LYS-SAF301 and LYS-SAF302 in an MPSIIIA mouse model showed that a gene therapy vector of the present disclosure transduced MPSIIIA brain cells to produce and secrete large quantities of active SGSH, which in turn, mediated highly significant reductions in primary, secondary and other neuropathology. Large quantities of SGSH were detected in some regions of the brain, and appeared to correlate directly with their proximity to the injection site. The data indicate that this vector is capable of producing sufficient quantities of SGSH in order to mediate highly significant improvements in primary and secondary storage pathology, in addition to downstream events, such as microgliosis.

It was discovered that the gene therapy vectors of the present disclosure provide unexpected advantages over those previously described, including high levels of SGSH expression in the brain following intracerebral injection. In addition, the methods of the present disclosure surprisingly resulted in a reduced immune response. These advantages correlated to increased efficacy. Furthermore, in certain embodiments, surgery time and time under anaesthesia are minimized, thus reducing associated risk to the patient. In addition, the compositions and methods of the present disclosure provide enhanced efficacy via improved expression of the therapeutic product, broader distribution of expression, and more efficient delivery via higher doses, larger volumes, and, increased injection flow rates.

Adeno-associated virus (AAV), a member of the Parvovirus family, is a small nonenveloped, icosahedral virus with single-stranded linear DNA genomes of 4.7 kilobases (kb) to 6 kb. AAV's life cycle includes a latent phase at which AAV genomes, after infection, are site specifically integrated into host chromosomes and an infectious phase in which, following either adenovirus or herpes simplex virus infection, the integrated genomes are subsequently rescued, replicated, and packaged into infectious viruses. The properties of non-pathogenicity, broad host range of infectivity, including non-dividing cells, and potential site-specific chromosomal integration make AAV an attractive tool for gene transfer.

To date, at least a dozen different serotypes of AAVs with variations in their surface properties have been isolated from human or non-human primates (NHP) and characterized.

The term “serotype” is a distinction with respect to an AAV having a capsid which is serologically distinct from other AAV serotypes. Serologic distinctiveness is determined on the basis of the lack of cross-reactivity between antibodies to one AAV serotype as compared to other AAV serotypes. The gene therapy vectors, also named vector, of the disclosure may have any one of the known serotypes (rh) of AVV, for example, any one of rh1, rh2, rh3, rh4, rh5, rh6, rh7, rh8, rh9 or rh10, preferably rh10. These various AAV serotypes may also be referred to as AAV1, AAV2, AAV3, AAV4, AAV5, AAV6, AAV7, AAV8, AAV9 or AAV10 (AAVrh.10).

In certain embodiments, vectors of the disclosure may have an artificial AAV serotype. Artificial AAV serotypes include, without limitation, AAVs with a non-naturally occurring capsid protein. Such an artificial capsid may be generated by any suitable technique, using a novel AAV sequence of the disclosure (e.g., a fragment of a vp1 capsid protein) in combination with heterologous sequences which may be obtained from another AAV serotype (known or novel), non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

AAV serotype rh.10 (AAVrh.10) is described in PCT Patent Application Publication No. WO 2003/042397. AAVrh.10 vectors have been shown to efficiently cross the blood-brain barrier and transduce neurons and astrocytes in the neonatal mouse central nervous system (Zhang, H., et al., Molecular Therapy 19, 1440-1448 (August 2011)). In addition, AAVrh.10 vectors has superior activity upon injection into the brain of rodents [10-12], and there is no natural disease with AAV serotype 10 in the human population.

The AAV genome is relatively simple, containing two open reading frames (ORFs) flanked by short inverted terminal repeats (ITRs). The ITRs contain, inter alia, cis-acting sequences required for virus replication, rescue, packaging and integration. The integration function of the ITR permits the AAV genome to integrate into a cellular chromosome after infection.

The nonstructural or replication (Rep) and the capsid (Cap) proteins are encoded by the 5′ and 3′ open reading frames (ORFs), respectively. Four related proteins are expressed from the rep gene; Rep78 and Rep68 are transcribed from the p5 promoter while a downstream promoter, p19, directs the expression of Rep52 and Rep40. Rep78 and Rep68 are directly involved in AAV replication as well as regulation of viral gene expression. The cap gene is transcribed from a third viral promoter, p40. The capsid is composed of three proteins of overlapping sequence; the smallest (VP-3) is the most abundant. Because the inverted terminal repeats are the only AAV sequences required in cis for replication, packaging, and integration, most AAV vectors dispense with the viral genes encoding the Rep and Cap proteins and contain only the foreign gene(s), e.g., therapeutic gene(s), inserted between the terminal repeats.

N-sulfoglycosamine sulfohydrolase (SGSH; also named sulfamidase) is the deficient protein involved in Sanfilippo type A syndrome (MPSIIIA, OMIM #252900). N-sulfoglucosamine sulfohydrolase (SGSH, EC 3.10.1.1, MIM 605270) belongs to the family of lysosomal hydrolases required for the stepwise degradation of a family of glycosaminoglycans called heparan sulfate (HS). SGSH is involved in the third step of heparan sulfate (HS) degradation. SGSH catalyses the hydrolysis of an N-linked sulphate from the non reducing terminal glucosaminide residue of HS.

In certain embodiments, the first generation gene therapy vectors of the present disclosure comprise polynucleotide sequences encoding both SGSH and SUMF1. The human sulfatase-modifying factor 1 cDNA (SUMF1, MIM 607939) encodes a protein that enhances sulfatase activity by post-translational modification. The reason for coexpressing SGSH with SUMF1 in the first generation product was twofold. Although SUMF1 is present in the cell, higher SGSH activities are obtained following gene transfer when the cofactor is also introduced. SUMF1 strongly enhances sulfatase activity when it is co-transfected with sulfatase cDNAs in wild type cultured cells and when co-delivered with a sulfatase cDNA via AAV vectors in cells from individuals affected by different diseases owing to sulfatase deficiencies [15-17]. Fraldi et al showed that co-delivery of SUMF1 and SGSH resulted in a synergic increase in SGSH activity associated with a reduction in lysosomal storage, and inflammatory markers in the brain using AAV2/5 vectors [13].

It was hypothesized that overexpression of SGSH may drastically reduce the availability of SUMF1 for other sulfatases, and therefore affect their function. This toxic process is illustrated in Multiple Sulfatase Deficiency caused by mutations in SUMF1 [15]. Levels of human endogenous SUMF1 are reportedly high in the kidney and liver, but very low in the brain [18-21]. In contrast, expression of endogenous SUMF1 is moderate in the mouse brain [22]. The amount of endogenous SUMF1 may become a limiting factor in cells and tissues. Therefore, it was believed to be advantageous to include SUMF1 in gene therapy vectors designed to over-express a sulfatase, such as those designed for MPSIIIA.

The present inventors found that the second generation vector described herein, SAF302, which does not include a polynucleotide expressing SUMF1, was capable of enhanced expression of SGSH and increased effectiveness in degrading HS, when compared to the first generation product that includes a polynucleotide expressing SUMF1.

In certain embodiments, a gene therapy vector of the present disclosure is an AAV serotype rh10 vector comprising a polynucleotide sequence encoding the human SGSH polypeptide or an active variant thereof. In certain embodiments, these gene therapy vectors may be administered to a subject in need thereof in a replication deficient AAVrh.10 vector comprising a defective AAV2 genome comprising a polynucleotide sequence encoding the human SGSH polypeptide or an active variant thereof driven by a promoter and packaged in capsid of AAVrh.10.

In certain embodiments, the gene therapy vector further comprises additional regulatory sequences, such as promoter sequences, enhancer sequences, and other sequences that contribute to accurate or efficient transcription or translation, such as an internal ribosome binding site (IRES) or a polyadenylation (polyA) sequence. In certain embodiments, the polynucleotide sequence encoding the human SGSH polypeptide or an active variant thereof is operably linked to the promoter sequence. In some embodiments, the gene therapy vector comprises a polyA sequence but does not comprise an IRES sequence.

In some embodiments, the present disclosure includes a replication deficient adeno-associated virus (AAV)-derived vector comprising a polynucleotide sequence, e.g., an expression cassette, comprising the following in 5′ to 3′order:

a promoter sequence;

a polynucleotide sequence encoding a human N-sulfoglucosamine sulfohydrolase (SGSH) polypeptide or an active variant thereof;

an internal ribosomal entry site (IRES) sequence;

a polynucleotide sequence encoding a human sulfatase-modifying factor 1 (SUMF1) polypeptide or an active variant thereof, and

a polyadenylation (polyA) sequence.

In certain embodiments, the vector further comprises AAV2 ITRs and an AAVrh.10 capsid or serotype.

The present disclosure also includes the use of the expression cassettes described herein but where the location of the polynucleotide sequence encoding a human N-sulfoglucosamine sulfohydrolase (SGSH) polypeptide or an active variant thereof and the polynucleotide sequence encoding a human sulfatase-modifying factor 1 (SUMF1) polypeptide or an active variant thereof are switched.

In particular embodiments, the present disclosure includes a replication deficient adeno-associated virus (AAV)-derived vector comprising a polynucleotide sequence, e.g., an expression cassette, comprising the following in 5′ to 3′order:

a promoter sequence;

a polynucleotide sequence encoding a human N-sulfoglucosamine sulfohydrolase (SGSH) polypeptide or an active variant thereof, and

a polyadenylation (polyA) sequence;

wherein the vector does not comprise a polynucleotide sequence encoding SUMF1 and does not comprise an IRES sequence.

In certain embodiments, the vector further comprises AAV2 ITRs and an AAVrh.10 capsid or serotype.

A variety of suitable promoter sequences, IRES sequences, and polyA sequences are known and available in the art, and any may be used according to the present disclosure.

In certain embodiments, the promoter is a constitutive promoter, an inducible promoter, a tissue specific promoter (e.g., a brain-specific or neural tissue- or neural cell-specific promoter), or a promoter endogenous to the subject. Examples of constitutive promoters include, without limitation, the CMV early enhancer/chicken R actin (CAG) promoter, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter [lnvitrogen]. In particular embodiments, the promoter is a mammalian PGK promoter, such as, e.g., a murine PGK promoter. In some embodiments, the promoter is the CAG promoter, wherein the CAG promoter carries a CMV IE Enhancer, CB promoter, CBA Exon 1, CBA intron, rabbit beta-intron, and rabbit beta-globin exon 2. The present disclosure provides an improved gene therapy vector comprising a polynucleotide sequence encoding SGSH or an active fragment thereof, wherein the PGK promoter of the first generation product has been replaced with a CAG promoter. In some embodiments, the CAG promoter is operably linked to the SGSH sequence. In some embodiments, the replacement of the PGK promoter with the CAG promoter surprisingly results in significantly enhanced expression of SGSH.

Examples of inducible promoters regulated by exogenously supplied promoters include the zinc-inducible metallothionine (MT) promoter, the dexamethasone (Dex)-inducible mouse mammary tumor virus (MMTV) promoter, the ecdysone insect promoter, the tetracycline-repressible system, and the tetracycline-inducible system. Inducible promoters and inducible systems are available from a variety of commercial sources, including, without limitation, lnvitrogen, Clontech and Ariad. Many other systems have been described and can be readily selected by one of skill in the art.

IRES (Internal Ribosome Entry Site) are structural RNA elements that allow the translation machinery to be recruited within the mRNA, while the dominant pathway of translation initiation recruits ribosomes on the mRNA capped 5′ end.

The poly(A) signal is used by the cell for the 3′ addition of a polyA tail onto the mRNA. This tail is important for the nuclear export, translation, and stability of mRNA. In some embodiments, the polyA unit is a human growth hormone 1 poly A unit.

In particular embodiments of vectors of the present disclosure, the promoter sequence is derived from CAG promoter sequence; and/or the polyA sequence is derived from a human growth hormone 1 polyA sequence.

In one embodiment, the expression cassette comprises in the following 5′ to 3′order:

a promoter sequence derived from a CAG promoter sequence;

a polynucleotide sequence encoding a human N-sulfoglucosamine sulfohydrolase (SGSH) polypeptide or an active variant thereof,
and

a polyA sequence derived from a human growth hormone polyA sequence.

In particular embodiments of vectors of the present disclosure, the expression cassette is flanked by two AAV internal terminal repeat (ITR) sequences, e.g., AAV2 ITRs, wherein one of the two AAV ITR sequences is located 5′ of the expression cassette and one of the two AAV ITR sequences is located 3′ of the expression cassette. ITR sequences comprise about 145 bases each. They were named so because of their symmetry, which was shown to be required for efficient multiplication of the AAV genome. Another property of these sequences is their ability to form a hairpin, which contributes to so-called self-priming and allows primase-independent synthesis of the second DNA strand. The AAV ITRs are the only cis-acting elements required for genome replication and packaging.

In one particular embodiment, the present disclosure includes a vector comprising a polynucleotide sequence comprising the following in 5′ to 3′order:

an AAV2 ITR sequence;

a CAG promoter sequence;

a polynucleotide sequence encoding a human N-sulfoglucosamine sulfohydrolase (SGSH) polypeptide or an active variant thereof;

a human growth hormone polyA sequence; and

an AAV2 ITR sequence.

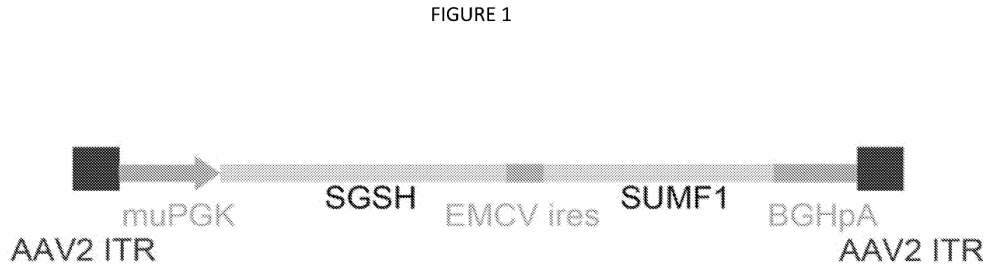

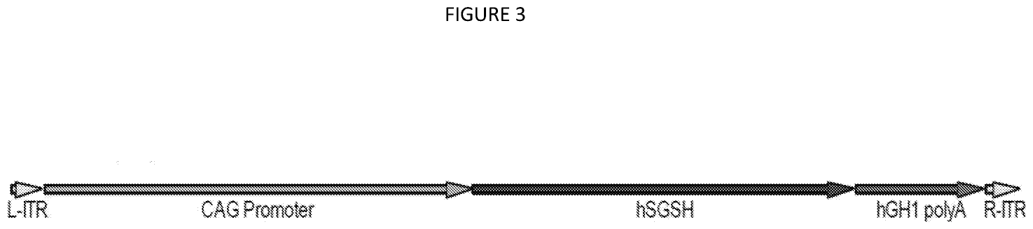

A schematic diagram of the first generation SAF301 vector is provided in FIG. 1. A schematic diagram of the vector construct of the present disclosure that is the second generation SAF302 vector is provided in FIG. 3.

The present disclosure further includes compositions, including pharmaceutical compositions, comprising a gene therapy vector of the present disclosure, as well as unit dosages thereof.

In one embodiment, the present disclosure includes a composition comprising a gene therapy vector described herein and a pharmaceutically acceptable carrier, diluent or excipient. Such a composition may be referred to as a pharmaceutical composition. In one particular embodiment, the pharmaceutically acceptable carrier, diluent, or excipient is a phosphate buffered saline solution, which may be sterile and/or GMP clinical grade. In one particular embodiment, a composition of the present disclosure comprises a vector comprising a polynucleotide sequence comprising the following in 5′ to 3′order:

an AAV2 ITR sequence;

a CAG promoter sequence;

a polynucleotide sequence encoding a human N-sulfoglucosamine sulfohydrolase (SGSH) polypeptide or an active variant thereof;

a human growth hormone 1 polyA sequence; and

an AAV2 ITR sequence,

wherein said vector is suspended in a phosphate buffered saline solution, which may be sterile and/or GMP clinical grade.

In certain embodiments, the concentration of vector present in a composition of the present disclosure is about 1×10¹⁰ gc/ml to about 1×10¹⁴ gc/ml, about 1×10¹¹ gc/ml to about 1×10¹³ gc/ml, or about 5×10¹¹ gc/ml to about 5 and 10¹² gc/ml. For example, in some embodiments, the concentration of vector present in the composition is from about 1.9×10¹² gc/mL to about 3.2×10¹² gc/mL, or about 2.4×10¹² gc/mL.

In particular embodiments, a unit dosage form of the present disclosure comprises a vial containing about 100 μl to 2 mL of a composition of the present disclosure. In certain embodiments, a unit dosage form comprises a vial containing about 1.2 mL of the composition. In particular embodiments, the amount of vector present in a unit dosage form is about 0.05×10¹² gc to about 3×10¹² gc, or about 0.1×10¹² gc to about 0.5×10¹² gc, or about 0.1×10¹² gc to about 2×10¹² gc.

Polynucleotide and Polypeptide Sequences

In certain embodiments, the present disclosure includes polynucleotide sequences comprising or consisting of an expression cassette described herein, as well as plasmids and vectors comprising any of the expression cassettes described herein. In addition, the disclosure includes cells comprising any of the polynucleotide sequences, vectors or plasmids of the present disclosure. One of skill in the art can readily produce polynucleotide sequence, vectors and host cells of the present disclosure using standard molecular and cell biology techniques and knowledge in the art.

The polynucleotide and polypeptide sequences of components of the polynucleotides, e.g., expression cassettes, of the present disclosure are known.

A murine PGK promoter sequence is provided at NCBI reference number MI8735 position+419-+924, and at position+419-+924 of SEQ ID NO:1.

An IRES sequence is provided at NCBI reference number NC001479 position+13-+575, and at position+13-+575 of SEQ ID NO:2.

A bovine growth hormone polyA sequence is provided at NCBI reference number M57764 position+2326-+2533, and at position+2326-+2533 of SEQ ID NO:3.

A CAG promoter sequence is provided as SEQ ID NO: 12 and/or as positions 125-1862 of SEQ ID NO: 9.

A human growth hormone 1 poly A sequence is provided as SEQ ID NO: 17 and/or as positions 3414-3923 of SEQ ID NO: 9.

N-sulfoglycosamine sulfohydrolase (SGSH; also named sulfamidase) is the deficient protein involved in Sanfilippo type A syndrome (MPSIIIA, OMIM #252900). N-sulfoglucosamine sulfohydrolase (SGSH, EC 3.10.1.1, MIM 605270) belongs to the family of lysosomal hydrolases required for the stepwise degradation of a family of glycosaminoglycans called heparan sulfate (HS). SGSH is involved in the third step of heparan sulfate degradation. SGSH catalyses the hydrolysis of an N-linked sulphate from the non reducing terminal glucosaminide residue of HS. A wild-type human N-sulfoglucosamine sulfohydrolase cDNA sequence is provided at NCBI reference number U30894 position+12-+1521, and at position+12-+1521 of SEQ ID NO:4. A wild-type human N-sulfoglucosamine sulfohydrolase polypeptide sequence is provided at NP_000190.1 (SEQ ID NO:5). A wild-type human sulfatase modifying factor 1 cDNA sequence is provided at NCBI reference number AY208752 position+1-+1125, and at position+1-+1125 of SEQ ID NO:6. A wild-type human sulfatase modifying factor 1 polypeptide sequence is provided at NP_877437.2 (SEQ ID NO:7). In particular embodiments, the SGSH and SUMF1 polypeptides include their signal sequences, while in other embodiments, one or both do not include the signal sequence. In some embodiments, the SGSH sequence is provided as SEQ ID NO: 13 and/or as amino acids 1867-3401 of SEQ ID NO: 9.

AAV cap sequences are known in the art. An exemplary AAVrh.10 cap polynucleotide sequence is provided as SEQ ID NO:59 in PCT Patent Application Publication No. WO2003/042397, with the sequence encoding VP1 at nucleotides 845-3061, VP2 at nucleotides 1256-3061, and VP3 at 1454-3061. An exemplary AAVrh.10 cap polypeptide sequence is provided as amino acid s 1-738 of SEQ ID NO:81 of PCT Patent Application Publication No. WO2003/042397, with the VP1 sequence at amino acids 1-738, VP2 at amino acids 138-738, and VP3 at amino acids 203-738.

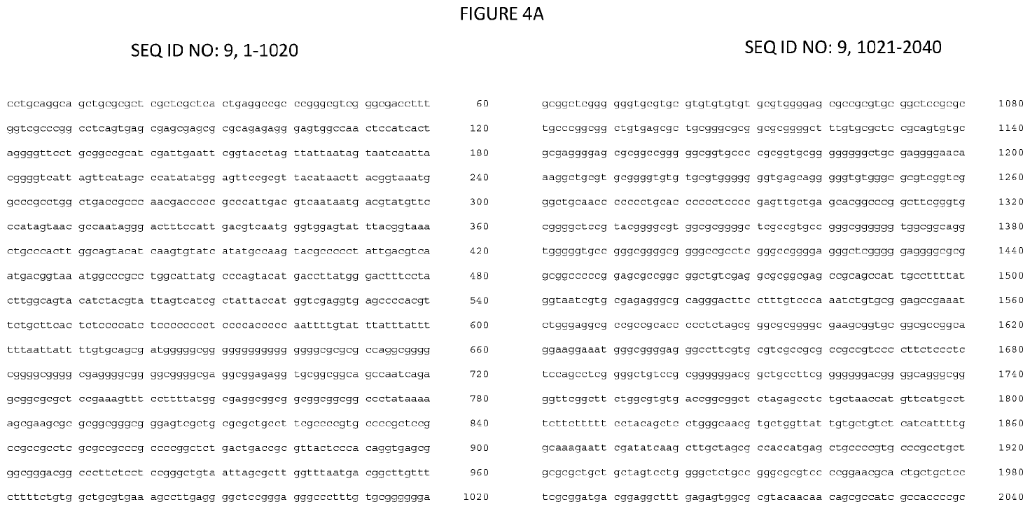

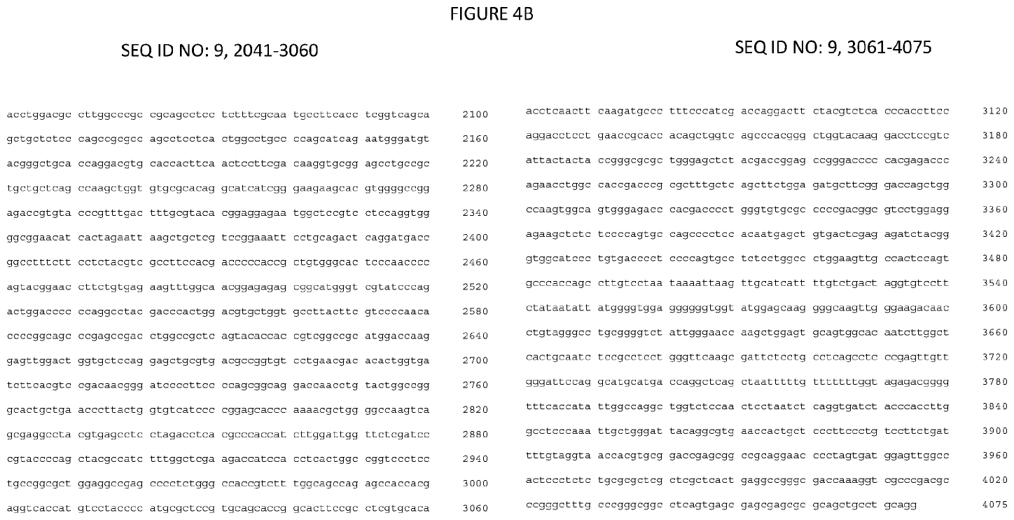

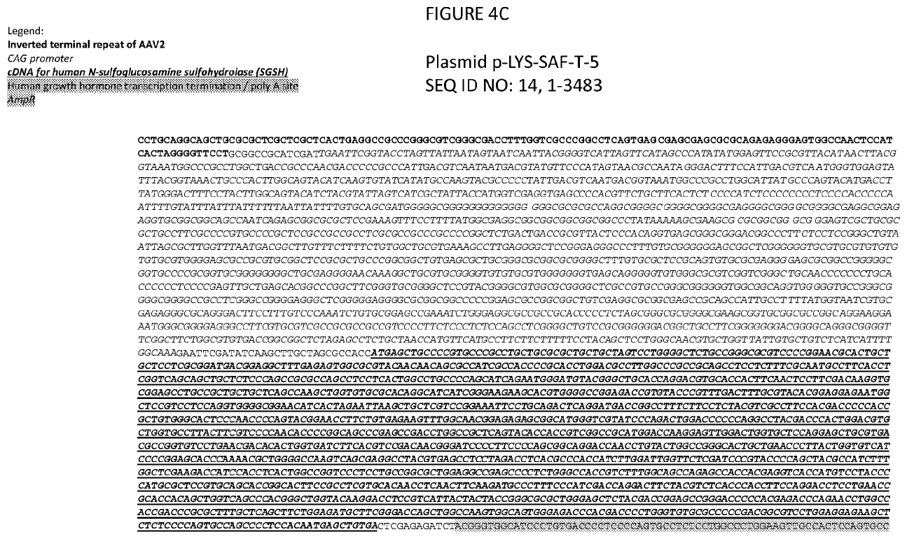

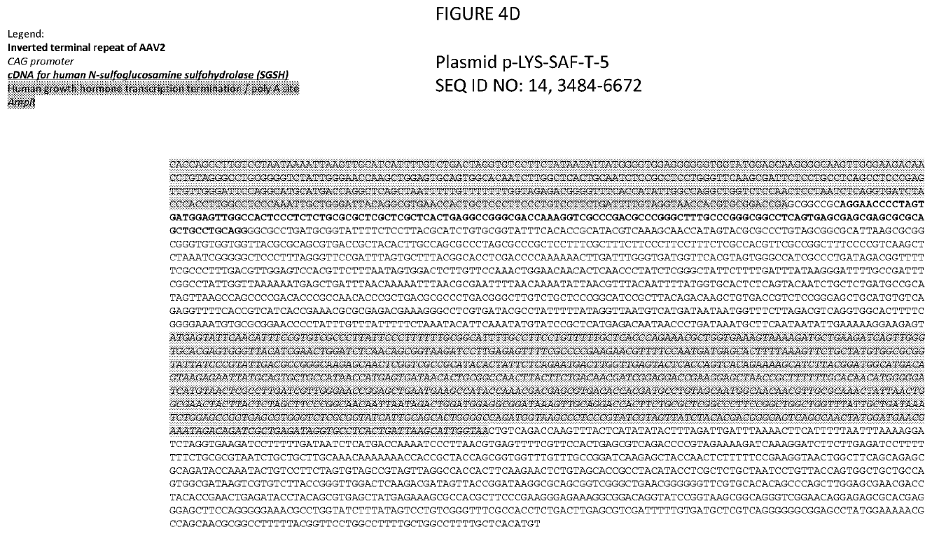

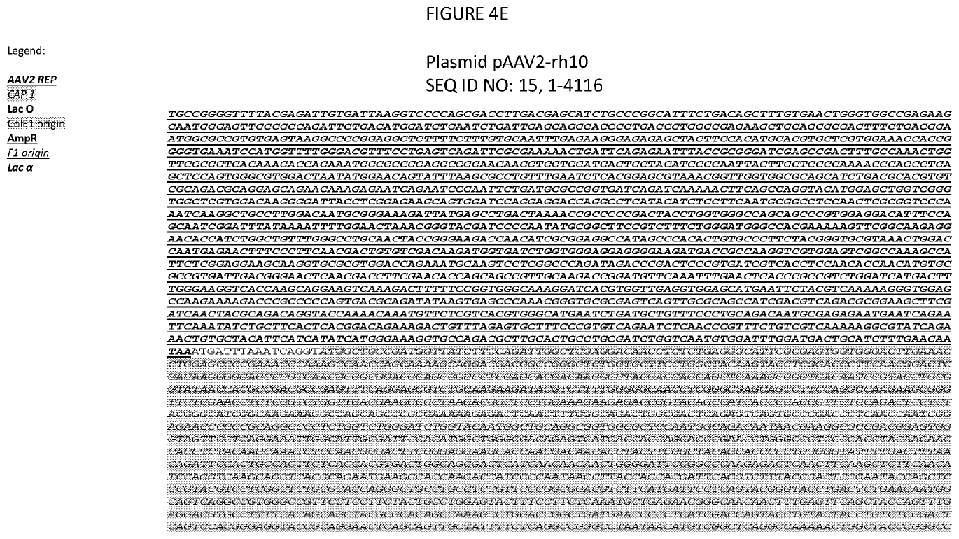

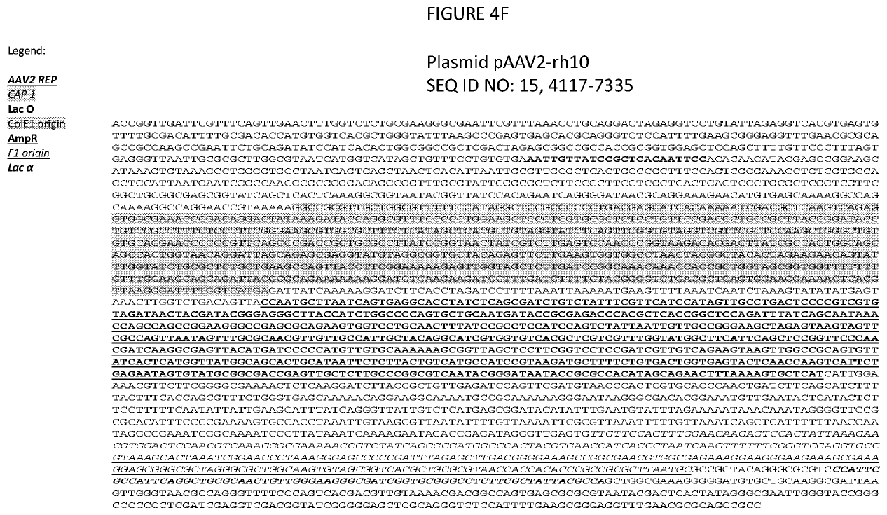

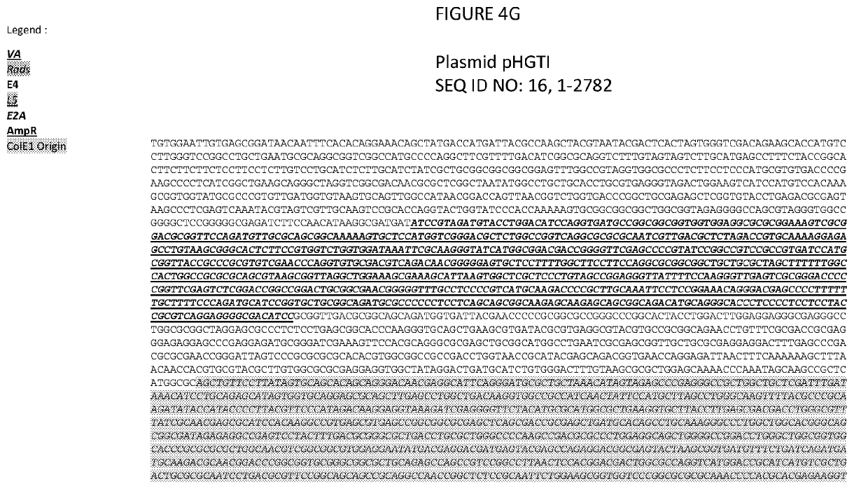

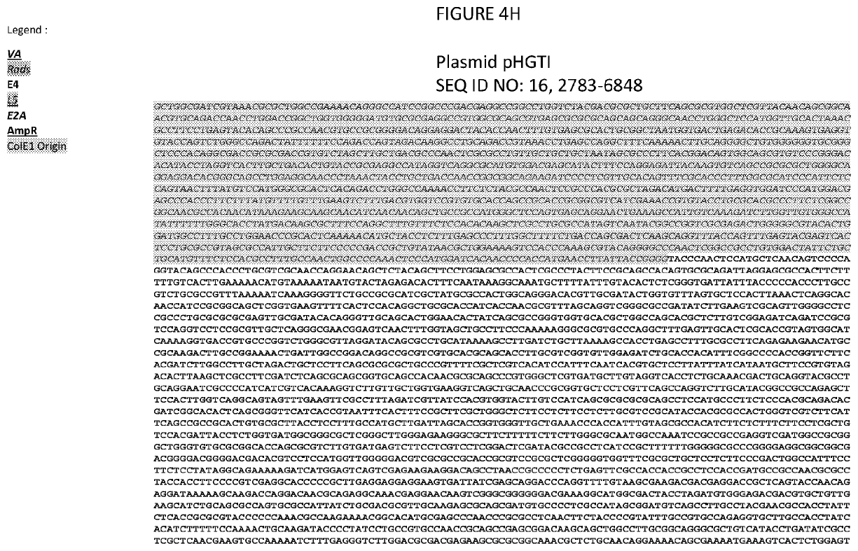

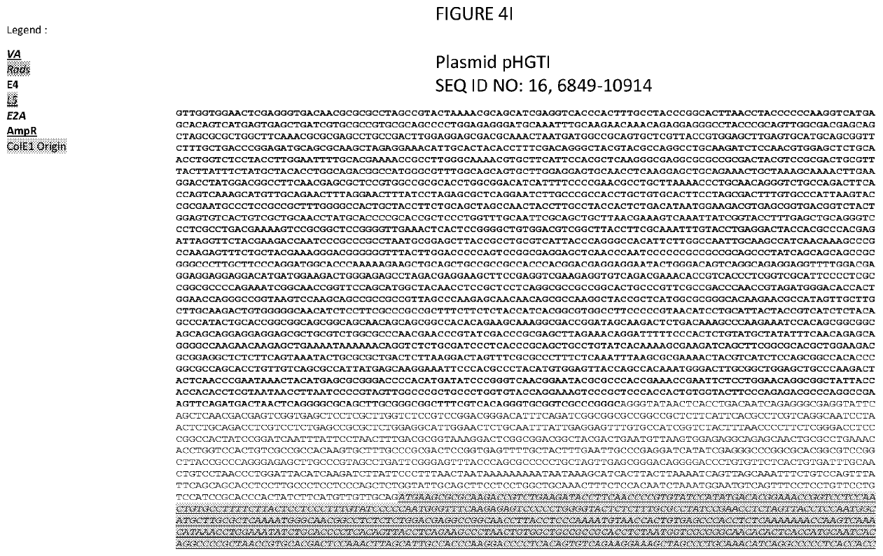

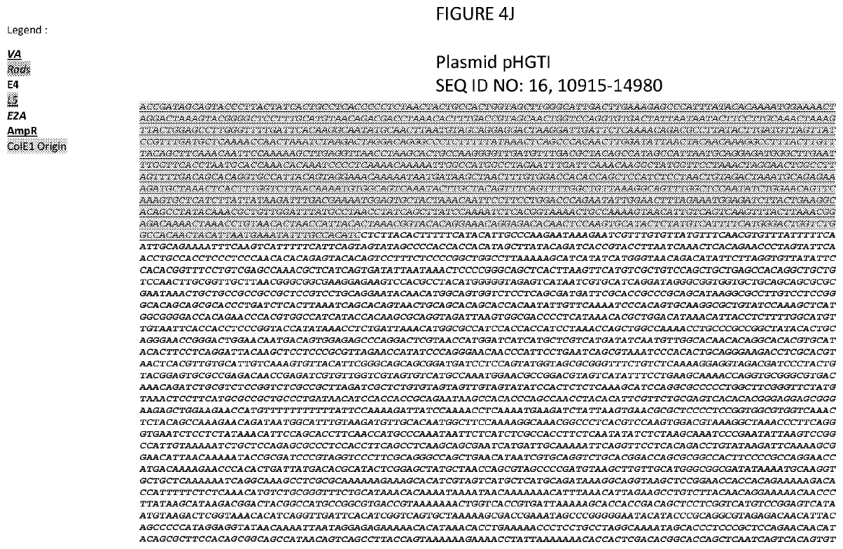

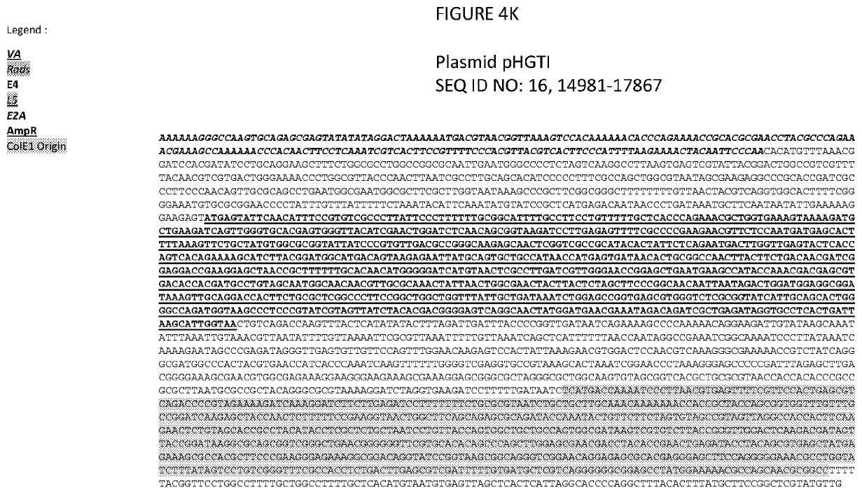

In some embodiments, the polynucleotide sequence of one expression cassette of the present disclosure and flanking ITRs as exemplified in FIG. 1 is provided as SEQ ID NO:8. In some embodiments, the polynucleotide sequence of one expression cassette of the present disclosure and flanking ITRs as exemplified in FIG. 3 is provided in FIGS. 4A-4B and SEQ ID NO: 9. In some embodiments, the polynucleotide of the full plasmid p-Lys-SAF-T-5, which contains SEQ ID NO: 9, is provided in FIGS. 4C-4D and SEQ ID NO: 14.

In certain embodiments, a polynucleotide sequence comprising an expression cassette is present in a vector or plasmid, e.g., a cloning vector or expression vector, to facilitate replication or production of the polynucleotide sequence. Polynucleotide sequences of the present disclosure may be inserted into vectors through the utilization of compatible restriction sites at the borders of the ITR sequences or DNA linker sequences which contain restriction sites, as well as other methods known to those skilled in the art. Plasmids routinely employed in molecular biology may be used as a backbone, such as, e.g., pBR322 (New England Biolabs, Beverly, Mass.), pRep9 (Invitrogen, San Diego, Calif.), pBS (Stratagene, La Jolla, Calif.) for the insertion of an expression cassette.

Vectors or plasmids of the present disclosure may be present in a host cell, e.g., in order to produce the gene therapy vector or viral particles for clinical use. In particular embodiments, the present disclosure includes a cell comprising a vector or plasmid comprising an expression cassette of the present disclosure. In particular embodiments, the host cell is a 293 human embryonic kidney cell, such as, e.g., a 293T cell, a highly transfectable derivative of 293 cell that contains the SV40 T antigen. Examples of other vectors, host cells, and methods of producing viral vectors are described in Kotin R M, Hum Mol Genet, 2011 Apr. 15; 20(R1):R2-6. Epub 2011 Apr. 29).

In additional embodiments, the present disclosure includes gene therapy vectors or viral particles comprising any of the expression cassettes of the present disclosure, wherein said gene therapy vector or viral particle comprises a capsid, e.g., an AAVrh.10 capsid. In particular embodiments, the capsid comprises one or more AAVrh.10 capsid polypeptides.

In certain embodiments, polynucleotides, expression cassettes and vectors of the present disclosure may include an active variant of one or more active polynucleotide or polypeptide sequences, such as an active variant of a promoter sequence, an IRES sequence or a polyA sequence, or an active variant of an SGSH polypeptide or a SUMF1 polypeptide. Active variants include both biologically active variants and biologically active fragments of any of the sequences provided herein, which may be referred to as reference sequences. In particular embodiments, active variants of a reference polynucleotide or polypeptide sequence have at least 40%, 50%, 60%, 70%, generally at least 75%, 80%, 85%, usually about 90% to 95% or more, and typically about 97% or 98% or 99% or more sequence similarity or identity to the reference polynucleotide or polypeptide sequence, as determined by sequence alignment programs described elsewhere herein using default parameters. For example, in some embodiments, the present disclosure provides a polynucleotide having at least about 75%, 80%, 85%, 90%, 95%, 96%, 97%, 98%, or 99% sequence identity to any sequences provided herein, such as SEQ ID NOs: 9, 10, 11, 12, 13, 14, 15, 16, and/or 17.

In certain embodiments, an active variant of a polynucleotide sequence encoding SGSH or SUMF1 varies from a wild-type or naturally occurring gene or cDNA sequence due to degeneracy of the genetic code. Accordingly, while the polynucleotide sequence is varied from wild-type, the encoded SGSH or SUMF1 polypeptide retains the wild-type sequence. Thus, the present disclosure contemplates the use of any polynucleotide sequence that encodes SGSH or SUMF1 polypeptides or active variants therein.

In other embodiments, an active variant of a polynucleotide sequence that is active itself, e.g., an IRES or polyA sequence, may vary in sequence from its corresponding wild-type reference sequence, although it retains its native activity. An active variant of a reference polynucleotide sequence may differ from that sequence generally by as much 200, 100, 50 or 20 nucleotide residues, or suitably by as few as 1-15 nucleotide residues, as few as 1-10, such as 6-10, as few as 5, as few as 4, 3, 2, or even 1 nucleotide residue.

In certain embodiments, active variants of polypeptides are biologically active, that is, they continue to possess an enzymatic activity of a reference polypeptide. Such variants may result from, for example, genetic polymorphism and/or from human manipulation. An active variant of a reference polypeptide may differ from that polypeptide generally by as much 200, 100, 50 or 20 amino acid residues, or suitably by as few as 1-15 amino acid residues, as few as 1-10, such as 6-10, as few as 5, as few as 4, 3, 2, or even 1 amino acid residue. In some embodiments, a variant polypeptide differs from the reference sequences referred to herein by at least one but by less than 15, 10 or 5 amino acid residues. In other embodiments, it differs from the reference sequences by at least one residue but less than 20%, 15%, 10% or 5% of the residues.

A reference polypeptide may be altered in various ways including amino acid substitutions, deletions, truncations, and insertions to produce an active variant. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, polypeptide variants contain conservative amino acid substitutions at various locations along their sequence, as compared to a reference polypeptide sequence. A “conservative amino acid substitution” is one in which the amino acid residue is replaced with an amino acid residue having a similar side chain. Families of amino acid residues having similar side chains have been defined in the art, which can be generally sub-classified as follows:

acidic: the residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid; basic: the residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine;

charged: the residues are charged at physiological pH and, therefore, include amino acids having acidic or basic side chains (i.e., glutamic acid, aspartic acid, arginine, lysine and histidine);

hydrophobic: the residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan; and

neutral/polar: the residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table 1.

TABLE 1
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine, |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant polypeptide can readily be determined by assaying its enzymatic activity, as described herein. Conservative substitutions are shown in Table 2 under the heading of exemplary substitutions. Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary Amino Acid Substitutions

| Original Residue | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Thus, a predicted non-essential amino acid residue in a reference polypeptide is typically replaced with another amino acid residue from the same side chain family. A “non-essential” amino acid residue is a residue that can be altered from the wild-type sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of wild-type. An “essential” amino acid residue is a residue that, when altered from the wild-type sequence of a reference polypeptide, results in abolition of an activity of the parent molecule such that less than 20% of the wild-type activity is present. For example, such essential amino acid residues may include those that are conserved in the enzymatic sites of reference polypeptides from various sources.

In certain embodiments, the present disclosure also contemplates active variants of naturally-occurring reference polypeptide sequences, wherein the variants are distinguished from the naturally-occurring sequence by the addition, deletion, or substitution of one or more amino acid residues. In certain embodiments, an active variant of a polypeptide includes an amino acid sequence having at least about 50%, 55%, 60%, 65%, 70%, 75%, 80%, 85%, 90%, 91%, 92%, 93%, 94% 95%, 96%, 97%, 98% or more sequence identity or similarity to a corresponding sequence of a reference polypeptide described herein, and retains an enzymatic activity of that reference polypeptide.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

Method for Producing Gene Therapy Vectors

Gene therapy vectors of the present disclosure may be produced by methods known in the art and previously described, e.g., in PCT Patent Application Publication No. WO03042397 and U.S. Pat. No. 6,632,670.

The AAV genome is a single-stranded deoxyribonucleic acid (ssDNA), either positive- or negative-sensed, which is about 4.7 kilobase long. The genome comprises ITRs at both ends of the DNA strand and two open reading frames (ORFs): rep and cap. Rep comprises four overlapping genes encoding Rep proteins required for the AAV life cycle, and cap comprises overlapping nucleotide sequences encoding capsid proteins: VP1, VP2 and VP3, which interact to form a capsid of an icosahedral symmetry.

The ITRs are believed to be required for both integration of the AAV DNA into the host cell genome and rescue from it, as well as for efficient encapsidation of the AAV DNA and generation of a fully-assembled AAV particles. With regard to gene therapy, ITRs seem to be the only sequences required in cis next to the therapeutic gene, and the structural (cap) and packaging (rep) genes can be delivered in trans. Accordingly, certain methods established for production of recombinant AAV (rAAV) vectors containing a therapeutic gene involve the use of two or three plasmids. In particular embodiments, the first plasmid comprises an expression cassette comprising a polynucleotide sequence encoding the therapeutic polypeptide, which contains flanking ITRs. In some embodiments, the second plasmid comprises rep and cap genes and flanking ITRs. In some embodiments, a third plasmid provides helper functions (e.g., from adenovirus serotype 5). In order to generate recombinant AAV vector stocks, standard approaches provide the AAV rep and cap gene products on a plasmid that is used to cotransfect a suitable cell together with the AAV vector plasmid encoding the therapeutic polypeptide. In some embodiments, standard approaches provide the AAV rep and cap gene products on a plasmid that is used to cotransfect a suitable cell together with the AAV vector plasmid encoding the therapeutic polypeptide and together with the plasmid providing helper functions.

In particular embodiments, AAV rep and cap genes are provided on a replicating plasmid that contains the AAV ITR sequences. In some embodiments, the rep proteins activate ITR as an origin of replication, leading to replication of the plasmid. The origin of replication may include, but is not limited to, the SV40 origin of replication, the Epstein-Barr (EBV) origin of replication, the ColE1 origin of replication, as well as others known to those skilled in the art. Where, for example, an origin of replication requires an activating protein, e.g., SV40 origin requiring T antigen, EBV origin requiring EBNA protein, the activating protein may be provided by stable transfection so as to create a cell line source, e.g., 293T cells), or by transient transfection with a plasmid containing the appropriate gene.

In other embodiments, AAV rep and cap genes may be provided on a non-replicating plasmid, which does not contain an origin of replication. Such non-replicating plasmid further insures that the replication apparatus of the cell is directed to replicating recombinant AAV genomes, in order to optimize production of virus. The levels of the AAV proteins encoding by such non-replicating plasmids may be modulated by use of particular promoters to drive the expression of these genes. Such promoters include, inter alia, AAV promoters, as well as promoters from exogenous sources, e.g., CMV, RSV, MMTV, E1A, EF1a, actin, cytokeratin 14, cytokeratin 18, PGK, as well as others known to those skilled in the art. Levels of rep and cap proteins produced by these helper plasmids may be individually regulated by the choice of a promoter for each gene that is optimally suited to the level of protein desired.

Standard recombinant DNA techniques may be employed to construct the helper plasmids used to produce viral vector of the present disclosure (see e.g., Current Protocols in Molecular Biology, Ausubel., F. et al., eds, Wiley and Sons, New York 1995), including the utilization of compatible restriction sites at the borders of the genes and AAV ITR sequences (where used) or DNA linker sequences which contain restriction sites, as well as other methods known to those skilled in the art.

In one embodiment, gene therapy vector of the present disclosure is produced by the transfection of two or three plasmids into a 293 or 293T human embryonic kidney cell line. In some embodiments, DNA coding for the therapeutic gene is provided by one plasmid, and the capsid proteins (from AAVrh.10), replication genes (from AAV2) and helper functions (from adenovirus serotype 5) are all provided in trans by a second plasmid. In some embodiments, DNA coding for the therapeutic gene is provided by one plasmid, the capsid proteins (from AAVrh.10) and replication genes (from AAV2) are provided in trans by a second plasmid, and helper functions (from adenovirus serotype 5) are provided by a third plasmid. In particular embodiments, the first plasmid comprises an expression cassette of the present disclosure, including the flanking ITRs.

Following cell culture, the gene therapy vector is released from cells by freeze thaw cycles, purified by an iodixanol step gradient followed by ion exchange chromatography on Hi-Trap QHP columns. The resulting gene therapy vector may be concentrated by spin column. The purified vector may be stored frozen (at or below −60° C.), e.g., in phosphate buffered saline.

Characterization of the final formulated vector may be achieved through SDS-PAGE and Western blot for capsid protein, real time PCR for transgene DNA, Western analysis, in vivo and in vitro general and specific adventitious viruses, and enzymatic assay for functional gene transfer.

Methods of Treatment

The present disclosure includes methods of treating brain diseases and disorders, neurological diseases and disorders, and genetic diseases and disorders, including, but not limited to, lysosomal storage diseases, such as MPS.

In certain embodiments, the present disclosure provides a method of treating MPSIIIA (Sanfilippo syndrome) comprising providing to a subject in need thereof a composition comprising a gene therapy vector designed to express SGSH when taken up by cells of the subject. In particular embodiments, the composition further comprises a pharmaceutically acceptable carrier, excipient or diluent, e.g., phosphate-buffered saline. In certain embodiments, a subject is a mammal, such as a human. In particular embodiments, a subject has been diagnosed with MPSIIIA, e.g., through genetic testing to identify a mutation in the subject's N-sulfoglycosamine sulfohydrolase (sgsh) gene or by measuring SGSH activity from a biological sample obtained from the subject. In some embodiments, the methods provided herein restore at least about 5%, at least about 10%, at least about 15%, at least about 20%, at least about 25%, at least about 30%, or more of normal SGSH activity throughout the brain of the subject.

In particular embodiments, the gene therapy vector comprises any expression cassette of the present disclosure. Accordingly, in specific embodiments, the present disclosure includes a method of treating MPSIIIA by administering to a subject in need thereof a composition comprising a gene therapy vector comprising an expression cassette comprising the following in 5′ to 3′ order:

a promoter sequence;

a polynucleotide sequence encoding a human N-sulfoglucosamine sulfohydrolase (SGSH) polypeptide or an active variant thereof;

and

a polyadenylation (polyA) sequence.

In some embodiments, the gene therapy vector does not comprise a polynucleotide sequence encoding SUMF1, and does not comprise an IRES sequence.

In one embodiment, the expression cassette is flanked by ITRs, and the vector thus comprises in the following 5′ to 3′order:

an AAV2 ITR sequence;

a CAG promoter sequence;

a polynucleotide sequence encoding a human N-sulfoglucosamine sulfohydrolase (SGSH) polypeptide or an active variant thereof;

a human growth hormone polyA sequence; and

an AAV2 ITR sequence.

In particular embodiments, the SGSH polypeptide comprises or consists of the wild-type human SGSH polypeptide, and in a specific embodiment, the expression cassette and flanking ITRs comprises the polynucleotide sequence set forth in SEQ ID NO:9.

In certain embodiments, the composition comprising the gene therapy vector is administered to the subject's brain. For example, in some embodiments, the composition comprising the gene therapy vector is administered via direct injection into the brain parenchyma. In certain embodiments, it is administered intracerebrally. In one embodiment, it is administered directly into the brain by intracerebral injection. This technique allows targeting selective neuro-anatomical sites to control the vector delivery.

Without wishing to be bound to any particular theory, it is believed that upon injection into the brain parenchyma, the AAV vector particle will diffuse locally and may also be transported along axons to remote anatomical brain structures. The vector particles are internalised by neuronal, glial or microglial cells. Each of these cell types are deficient for the SGSH enzyme in MPSIIIA patients and suffer from the toxic accumulation of undegraded heparan sulphate catabolites. Upon entry into the cells, the recombinant genome encoding the SGSH protein is transported into the nucleus where it undergoes a series of molecular transformations that result in its stable establishment as a double stranded deoxyribonucleic acid (DNA) molecule. This DNA is actively transcribed into messenger ribonucleic acids (mRNAs) by the cellular machinery. The mRNAs are translated into SGSH, which will complement the cell deficiency.

Enzyme complementation and correction of lysosomal storage is thought to occur by two different mechanisms: (1) the enzyme may reach the lysosome of cells which contain and express the AAV-borne transgene and degrade the accumulated catabolites; or (2) the enzyme can be released outside these cells, recaptured by distant cells and routed towards their lysosome. Hence, a limited group of genetically modified cells allows for the correction of extended brain territories. Transduced cells will express and deliver the enzyme continuously, thus constituting an intracerebral permanent source of enzyme production.

In particular embodiments, intracerebral administration is performed at one or more, e.g., two, three, four, five, six, seven, eight, nine, ten, eleven, or twelve, sites in the subject's brain. Administration may be performed to one or both sides of the brain. For example, where the gene therapy vector is administered to two or more sites of the brain, it may be administered to both sides of the brain, e.g., at similar or the same locations on each side. In particular embodiments, the gene therapy vector is administered to one or more sites in the subject's brain selected from: anterior right, anterior left, medial right, medial left, posterior right, and posterior left.

In certain embodiments, intracerebral administration is performed through one or more burr holes, which may be in the white matter adjacent to the putamen. In particular embodiments, gene therapy vector is administered to two or more sites within the brain or white matter through a single burr hole. For instance, two or more deposits of gene therapy vector may be administered through a single burr hole or track, with one being deep and the other being superficial, which may enhance parenchymal diffusion. In certain embodiments, the deep injection is performed at a depth of about 1.5 cm to about 3.0 cm, or about 1.7 cm to about 2.5 cm, or about 2 cm from the cortical surface. In certain embodiments, the superficial injection is performed at a depth of about 0.5 cm to about 2.0 cm, or about 0.7 cm to about 1.5 cm, or about 1 cm from the cortical surface.

In certain embodiments, the gene therapy vector is administered via intracerebral injection, wherein the injection is performed at a single depth. In some embodiments, the gene therapy vector is administered via bilateral injections at a single depth. For example, in some embodiments, the gene therapy vector is administered via intracerebral injection to 2, 4, 6, 8, 10, 12, 14, 16, or more sites within the subject's brain, through 2, 4, 6, 8, 10, 12, 14, or 16 burr holes in the white matter, with a single deposit of gene therapy vector being administered through each burr hole or track. In some embodiments, the administration of the gene therapy vector at a single depth is surprisingly superior to administration of the gene therapy vector at more than one depth, such as at two depths. Without wishing to be bound by theory, vector spread is more efficient when the gene therapy vector is administered at a single depth compared to two or more depths.

In one specific embodiment, a gene therapy vector is administered via intracerebral injection to six sites within the subject's brain, through six burr holes in the white matter, with a single deposit of a gene therapy vector being administered through each burr hole or track. Thus, the gene therapy vector is administered at a single depth within each injection burr hole. The location of administration within each burr hole may be selected from: anterior right, anterior left, medial right, medial left, posterior right, and posterior left. In some embodiments, the location of administration within each burr hole may be selected from: anterior right superficial, anterior right deep, anterior left superficial, anterior left deep, medial right superficial, medial right deep, medial left superficial, medial left deep, posterior right superficial, posterior right deep, posterior left superficial, and posterior left deep. In some embodiments, a deep injection is performed at a depth of about 1.5 cm to about 3.0 cm, or about 1.7 cm to about 2.5 cm, or about 2 cm from the cortical surface; and a superficial injection is performed at a depth of about 0.5 cm to about 2.0 cm, or about 0.7 cm to about 1.5 cm, or about 1 cm from the cortical surface. In some embodiments, deep and/or superficial injections may be selected based on MRI images. In some embodiments, a combination of deep and superficial injections may be administered, such that each injection is administered at a single depth, and each injection is either a deep or superficial injection. In some embodiments, all injections administered to a subject are deep injections. In some embodiments, all injections administered to a subject are superficial injections.

Injections may be accomplished in a single neurosurgical session. Intracerebral injections may be performed using an infusion pump.

In various embodiments in this disclosure, the term or unit genome copies (gc) is used interchangeably with the term or unit viral genomes (vg).

In certain embodiments, a total of about 1.0×10¹⁰ gc to about 1.0×10¹⁴ gc, about 5.0×10¹⁰ gc to about 5.0×10¹³ gc, about 5.0×10¹⁰ gc to about 1.0×10¹³ gc, about 1.0×10¹¹ gc to about 1.0×10¹³ gc, about 1.0×10¹¹ gc to about 5.0×10¹² gc, about 5.0×10¹¹ gc to about 5.0×10¹² gc, about 8.0×10¹¹ gc to about 8.0×10¹², or about 7.0×10¹² gc to about 7.4×10¹² gc of viral vector is administered to the subject. In particular embodiments, about 7.2×10¹² gc of viral vector is administered to the subject. In certain embodiments, about 0.8×10⁹ gc to about 0.8×10¹³ gc, about 0.4×10¹⁰ gc to about 0.4×10¹³ gc, about 0.4×10¹⁰ gc to about 0.8×10¹² gc, about 0.8×10¹⁰ gc to about 0.8×10¹² gc, about 0.8×10¹⁰ gc to about 0.4×10¹² gc, or about 0.4×10¹¹ gc to about 0.4×10¹² gc of viral vector is administered to each site of the subject. In particular embodiments, about 0.6×10¹¹ gc of viral vector is administered to each site of the subject. In particular embodiments, about 0.6×10¹¹ gc of viral vector is administered to each of twelve sites in the subject's brain or white matter, such that about 7.2×10¹¹ gc of viral vector is administered to the subject. In particular embodiments, about 1.2×10¹² gc of viral vector is administered to each site of the subject. In particular embodiments, about 1.2×10¹² gc of viral vector is administered to each of six sites in the subject's brain or white matter, such that about 7.2×10¹² gc of total viral vector is administered to the subject. In some embodiments, the amount of viral vector delivered to the subject is from about 6.0×10⁹ to about 8.0×10⁹ gc per gram of brain tissue (gc/gr). In some embodiments, the amount of viral vector delivered to the subject is from about 7.0×10⁹ gc/gr to about 7.4×10⁹ gc/gr. In some embodiments, the amount of viral vector delivered to the subject is about 7.2×10⁹ gc/gr.

In some embodiments, the gene therapy vector is administered in a formulation comprising a PBS buffer. In some embodiments, the PBS buffer does not comprise any excipients or preservatives. In some embodiments, the composition of the PBS buffer comprises KCl, KH₂PO₄, NaCl, and/or Na₂HPO₄. In some embodiments, the composition of the PBS buffer comprises about 2.67 mM KCl, about 1.47 mM KH₂PO₄, about 137.9 mM NaCl, and about 8.06 mM Na₂HPO₄. In some embodiments, the pH of the formulation is about 6.8 to about 7.8, or about 7.2-7.4.

In certain embodiments, the volume of composition comprising the gene therapy vector that is administered to each site is about 10 μl to about 600 μl, about 20 μl to about 550 μl, about 30 μl to about 200 μl, about 50 μl, about 100 μl, or about 150 μl. In some embodiments, the volume of the composition comprising the gene therapy vector that is administered to each site in each injection is about 200 μl, about 300 μl, about 400 μl, about 500 μl, about 600 μl. In certain embodiments, the volume of each injection is about 500 μl. In particular embodiments, the infusion rate for administration of the composition comprising the gene therapy vector is about 0.1 μl/min to about 10 μl/min, about 0.2 μl/min to about 8 μl/min, about 0.3 μl/min to about 6 μl/min, or about 0.5 μl/min to about 5 μl/min, or about 0.4 μl/min to about 4.0 μl/min, or about 0.4 μl/min to about 3.0 μl/min or about 2.0 μl/min. In some embodiments, the infusion rate for administration of the composition is about 5 μl/min. In some embodiments, the composition comprising the gene therapy vector is administered via multiple injections of about 500 μl, at a flow rate of about 5 μl/min.

Dosing selection in mammals, including humans, may be based on the following efficacy and safety criteria: (1) the total amount of vector particle delivered to the entire brain should be sufficient to induce SGSH production in a significant volume of the brain and should prevent development of storage lesions in the brain; and (2) the amount of vector particles at each deposit must not induce local toxicity.

Previous preclinical studies on other MPS models provided some information concerning efficacy. Doses of AAV vectors have been evaluated in canine models of MPS (MPSI and MPSIIIB), following stereotaxic injections into the brain. These studies have concluded that 8 deposits of 40 μl×1.5×10¹² vg/ml, representing 6×10¹⁰ vg at each deposit were sufficient for efficient vector delivery in the entire dog brain. A 4-fold higher dosing was safe, although it may not be more efficient with respect to vector delivery in brain tissue. This characterizes the dose of 6×10¹⁰ vg per deposit as safe and efficient.

In one particular embodiment, the present disclosure provides a method of treating MPSIIIA, said method comprising administering to a subject in need thereof (e.g., a human diagnosed with MPSIIIA) a composition comprising a viral vector comprising an expression cassette comprising the following sequence in 5′ to 3′ order, wherein the CAG promoter sequence is operably linked to the polynucleotide sequence encoding SGSH: an AAV2 ITR sequence;

a CAG promoter sequence;

a polynucleotide sequence encoding a human N-sulfoglucosamine sulfohydrolase (SGSH) polypeptide or an active variant thereof;

a human growth hormone polyA sequence; and

an AAV2 ITR sequence,

wherein said composition is administered via intracerebral injection to six sites within the subject's brain through six burr holes in the subject's head,

wherein the composition is administered to a single site and at a single depth through each burr hole or track,

wherein the six sites are anterior right, anterior left, medial right, medial left, posterior right, and posterior left,

wherein about 0.8×10⁹ to 0.8×10¹³ gc or about 1.2×10¹² gc of viral vector is administered to each of the twelve sites,

wherein the volume of composition administered to each of the twelve sites is about 10 μl to about 600 μl, or about 500 μl, and

wherein the infusion rate for administration of the composition is about 0.1 μl/min to about 10 μl/min, about 0.2 μl/min to about 8 μl/min, about 0.3 μl/min to about 6 μl/min, or about 0.4 μl/min to about 4.0 μl/min, about 0.4 μl/min to about 3.0 μl/min, or about 2.0 μl/min, or about 5.0 μl/min.

In certain embodiments, administration is performed using an infusion pump.

The methods for treating MPSIIIA are advantageous for delivery of therapeutic agents to the brain and nervous system, and they may be modified to treat other brain diseases or disorders or neurological diseases or disorders by using a different therapeutic agent.

As further described in the accompanying examples, the gene therapy vector provided herein, LYS-SAF302, provided significant improvements over gene therapy vectors such as LYS-SAF301. For example, in some embodiments, LYS-SAF302 resulted in superior expression and potency in producing SGSH and decreasing the amount of HS in the brain. LYS-SAF302 could safely and effectively be dosed at a total dose of 7.2×10¹² vg, at an infusion rate of about 5.0 μl/min. For example, LYS-SAF302 dosed in 6 administrations of 500 μL volume injections at a single depth per injection and 5.0 μl/min provides a highly effective, safe treatment for neurological diseases involving mutations in the SGSH gene.

Accordingly, in certain embodiments, the present disclosure includes a method of treating a brain or neurological disease or disorder resulting from a mutated gene in a subject in need thereof, comprising intracerebral administration to the subject of a gene therapy vector comprising an expression cassette comprising a polynucleotide sequence encoding the polypeptide encoded by the gene in its wild-type or non-mutated form, or an active variant thereof, wherein said polynucleotide sequence is operably linked to a promoter sequence, and wherein said intracerebral administration comprises administering about 5×10¹⁰ gc to about 5×10¹³ gc, or about 1.0×10¹¹ gc to about 1.0×10¹³ gc, in about six to about twelve dosages, each dosage totaling about 5.0×10⁹ gc to about 5.0×10¹² gc, or about 1.0×10¹⁰ gc to about 1.0×10¹² gc, or about 6×10¹⁰ gc, in a volume of about 10 μl to about 500 μl. In certain embodiments, the polynucleotide sequence is operably linked to a CAG promoter. In certain embodiments, each dosage is administered at a rate of about 0.1 μl/min to about 10 μl/min, about 0.2 μl/min to about 8 μl/min, about 0.3 μl/min to about 6 μl/min, or about 0.4 μl/min to about 4.0 μl/min, about 0.5 μl/min to about 5.0 μl/min, about 2.0 μl/min, or about 5 μL/min. In particular embodiments, the intracerebral administration is performed using a delivery device, optionally comprising a catheter. In certain embodiments, the intracerebral administration comprises administration of the vector to the brain or white matter of the subject through six holes in the white matter adjacent to the putamen. In particular embodiments, two of the twelve dosages are administered through each of the six holes, wherein one of the two dosages is a deep administration and one of the two dosages is a superficial administration. In certain embodiments, each of the twelve dosages are administered via catheter. In one embodiment, the intracerebral administration is performed using an infusion pump. In some embodiment, the intracerebral administration comprises administration of the vector to the brain or white matter of the subject in six dosages, wherein each of the dosages is administration at a single depth.

The present disclosure further includes a method of treating a brain or nervous system disease or disorder in a subject in need thereof, or a method of providing a therapeutic agent to the brain of a subject in need thereof, said method comprising administering to a subject in need thereof (e.g., a human) a composition comprising a therapeutic agent, wherein said composition is administered via intracerebral injection to six or twelve sites within the subject's brain through six burr holes in the subject's head. In some embodiments, the composition is administered to two sites through each burr hole or track, wherein one of the two sites is deep and the other of the two sites is superficial, wherein the twelve sites are anterior right superficial, anterior right deep, anterior left superficial, anterior left deep, medial right superficial, medial right deep, medial left superficial, medial left deep, posterior right superficial, posterior right deep, posterior left superficial, and posterior left deep, wherein the volume of composition administered to each of the twelve sites is about 10 μl to about 300 μl, and wherein the infusion rate for administration of the composition is about 0.1 μl/min to about 10 μl/min, about 0.2 μl/min to about 8 μl/min, about 0.3 μl/min to about 6 μl/min, or about 0.4 μl/min to about 4.0 μl/min, about 0.5 μL to about 5.0 μL, about 0.4 μl/min to about 3.0 μl/min, or about 2.0 μl/min, or about 5.0 μL/min. In certain embodiments, administration is performed using an infusion pump. In some embodiments, the composition is administered to the burr holes in the subject's head, wherein the composition is administered to a single site through each burr hole or track. In some embodiments, the single burr hole is selected from a deep or a superficial site. For example, in some embodiments, each of the administrations is a deep site. In some embodiments, each of the administrations is a superficial site. In some embodiments, the multiple administrations are a mixture of deep and superficial sites, that is, each site of administration is independently selected for deep or superficial administration.

The disclosure contemplates the use of any type of therapeutic agent, including but not limited to small molecules, polypeptides, antibodies and fragments thereof, polynucleotides, e.g., siRNA, antisense RNA, miRNA, and viral vectors. In particular embodiments, the therapeutic agent is a viral vector, e.g. an AAV vector, such as an AAVrh.10 vector.

In particular embodiments, this method is used to treat a lysosomal storage disorder caused by a genetic defect, including but not limited to an MPS (including but not limited to Sanfilippo C, Sanfilippo D, Sl, Hurler-Scheie, Sanfilippo A, Hunter, Morquio, Sanfillippo B, Maroteaux-Lamy), Gaucher, Metachromatic Leukodystrophy, Fabry, Krabbe, Pompe, Cystinosis, Tay-Sachs, Niemann Pick C, Niemann Pick A/B, Mucolipidosis II/III, Gm1 Gangliosidosis, Sandhoff, or any other described herein, wherein the therapeutic agent is a viral vector that provides a functional or wild-type version of a missing or mutated enzyme associated with the particular disease. The enzyme may be provided in an AAV viral vector such as any of those described herein, but with the SGSH encoding polynucleotide sequence (and optionally IRES) replaced by a polynucleotide sequence encoding the desired enzyme. The genetic basis for these diseases is known, and such enzymes and polynucleotide sequences are known and available in the art. For example, the enzyme associated with MPSII is iduronate-2-sulfatase; the enzyme associated with MPS1 is alpha-L-iduronidase, the enzyme associated with MPSIIID is glucosamine-6-sulfatase, the enzyme associated with MPSIIIC is N-acetyltransferase; the enzyme associated with MPSIIIB is alpha-N-acetylglucosaminidase or glucuronate-2-sulphatase, and the enzyme associated with MPSVII is beta-D-glucuronidase or glucosamine-3-sulphatase.

Other brain or neurological diseases and disorders may also be treated by delivering a therapeutic agent to the brain according to the method described herein, including but not limited to any described herein.

### Methods for Treatment Using a Combination of Gene Therapy and Immunosuppressants

The present disclosure also includes methods of treating genetic disorders with a gene therapy vector in combination with one or more immunosuppressants. It is a surprising and unexpected finding of the present disclosure that long-term treatment with immunosuppressants following administration of a gene therapy vector enhances efficacy of the gene therapy treatment by reducing the inflammatory or immune response to the gene therapy vector.

In certain embodiments, the present disclosure includes a method of treating MPSIIIA comprising administering to a subject in need thereof:
(1) a gene therapy vector comprising an expression cassette comprising a polynucleotide sequence encoding a SGSH polypeptide or active variant thereof, wherein said polynucleotide sequence is operably linked to a promoter sequence; and
(2) one or more immunosuppressants,
   wherein at least one of the one or more immunosuppressant is provided to the subject for a duration of time of at least the two months, at least the three months, at least the four months, at least the five months, at least the six months, at least the eight months, at least the ten months, or at least the twelve months immediately following administration of the gene therapy vector. In certain embodiments, at least one of the one or more immunosuppressants is provided to the subject for the remainder of the subject's life, or for as long as the subject is producing a detectable level of SGSH polypeptide from the expression cassette.

In particular embodiments, any of the gene therapy vectors of the present disclosure described herein may be used to practice the method.

In one particular embodiment, the present disclosure includes a method of treating MPSIIIA in a subject in need thereof, said method comprising: administering to a subject in need thereof (e.g., a human diagnosed with MPSIIIA):
(1) a composition comprising a viral vector comprising an expression cassette comprising the following sequences in order from 5′ to 3′, wherein the CAG promoter sequence is operably linked to the polynucleotide sequence encoding SGSH:
   a. an AAV2 ITR sequence;
   b. a CAG promoter sequence;
      a polynucleotide sequence encoding a human N-sulfoglucosamine sulfohydrolase (SGSH) polypeptide or an active variant thereof;
   c. a human growth hormone polyA sequence; and
   d. an AAV2 ITR sequence; and
(2) one or more immunosuppressants,
   wherein at least one of the one or more immunosuppressant is provided to the subject for a duration of time of at least the two months, at least the three months, at least the four months, at least the five months, at least the six months, at least the eight months, at least the ten months, or at least the twelve months immediately following administration of the gene therapy vector. In certain embodiments, at least one of the one or more immunosuppressants is provided to the subject for the remainder of the subject's life, or for as long as the subject is producing a detectable level of SGSH polypeptide from the expression cassette.

In particular embodiments of these methods, these methods include one or more of the following features: said composition is administered via intracerebral injection to the subject's brain through burr holes in the subject's head; the composition is administered via four to eight burr holes; the composition is administered to one or two sites through each burr hole or track, wherein the sites are independently either deep and or superficial; the sites are selected from anterior right superficial, anterior right deep, anterior left superficial, anterior left deep, medial right superficial, medial right deep, medial left superficial, medial left deep, posterior right superficial, posterior right deep, posterior left superficial, and posterior left deep, a dosage of about 0.8×10⁹ gc to about 0.8×10¹³ gc, about 1.0×10¹⁰ gc to about 1.0×10¹³, or about 1.0×10¹⁰ gc to about 1.0×10¹² gc or about 6×10¹⁰ gc or about 1.2×10¹² gc of viral vector is administered to each of the six or twelve sites,

wherein the volume of composition administered to each of the twelve sites is about 10 μl to about 600 μl, and/or wherein the infusion rate for administration of the vector is about 0.1 μl/min to about 10 μl/min, about 0.2 μl/min to about 8 μl/min, about 0.3 μl/min to about 6 l/min, or about 0.4 μl/min to about 4.0 μl/min, about 0.4 μl/min to about 3.0 μl/min or about 2.0 μl/min, or about 5.0 μl/min. In certain embodiments, administration is performed using an infusion pump. In certain embodiments, the composition is administered to all twelve of the sites recited above. In one embodiment, the method includes all of the features recited above.

In particular embodiments of methods that include providing one or more immunosuppressants, the one or more immunosuppressants comprises a calcineurin inhibitor (e.g., tacrolimus), a macrolide (e.g. sirolimus or rapamicyn), and/or mycophenolate mofetil. In certain embodiments, at least one of the one or more immunosuppressant is provided to the subject for a duration of time of at least the three months, at least the four months, at least the five months, at least the six months, at least the eight months, at least the ten months, or at least the twelve months immediately following administration of the gene therapy vector. In certain embodiments, at least one of the one or more immunosuppressants is provided to the subject for a duration of time prior to administration of the gene therapy vector.

In one particular embodiment, the one or more immunosuppressants comprises a calcineurin inhibitor, e.g., tacrolimus, which is provided to the subject for a duration of time of at least the twelve months immediately following administration of the gene therapy vector. In one particular embodiment, the one or more immunosuppressants comprises a macrolide, e.g., sirolimus, which is provided to the subject for a duration of time of at least the twelve months immediately following administration of the gene therapy vector. In certain embodiments, the one or more immunosuppressants comprise a calcineurin inhibitor (e.g., tacrolimus) and mycophenolate mofetil. In certain embodiments, the one or more immunosuppressants comprise a macrolide (e.g., sirolimus) and mycophenolate mofetil. In particular embodiments, the calcineurin inhibitor or macrolide is provided to the subject for a duration of time of at least the twelve months immediately following administration of the gene therapy vector, and the cycophenolate mofetil is provided to the patient for about two months, beginning about two weeks before administration of the gene therapy vector.

In another particular embodiment, the calcineurin inhibitor is tacrolimus, which is provided to the subject at a dosage of 0.05 mg/kg/day-1.0 mg/kg/day or about 0.2 mg/kg/day for at least the two months or at least the three months immediately following administration of the gene therapy vector. In another particular embodiment, the calcineurin inhibitor is tacrolimus, which is provided to the subject at a dosage that achieves a blood concentration of tacrolimus of 10 ng/ml-15 ng/ml for a period of time during the three months immediately following administration of the gene therapy vector. In a further particular embodiment, the calcineurin inhibitor is tacrolimus, which is provided to the subject at a dosage that achieves a blood concentration of tacrolimus of 7 ng/ml-10 ng/ml for a period of time during the fourth month through the twelfth month immediately following administration of the gene therapy vector. In particular embodiments, the calcineurin inhibitor, e.g., tacrolimus, is provided to the subject for up to one year, up to two years, up to three years, up to four years, up to five years, or for the duration of the subject's life following administration of the gene therapy vector.

In another embodiment, the macrolide is sirolimus, which is provided to the subject at a dosage of 3 mg/kg/day or about 1.5 mg/kg/twice per day for at least the two months or at least the three months immediately following administration of the gene therapy vector. In another particular embodiment, the macrolide is sirolimus, which is provided to the subject at a dosage that achieves a blood level of sirolimus of 10 ng/ml-25 ng/ml for a period of time during the three months immediately following administration of the gene therapy vector. In a further particular embodiment, the one or more immunosuppressants is sirolimus, which is provided to the subject at a dosage that achieves a blood level of sirolimus of 20 ng/ml for a period of time during the second month through the sixth month immediately following administration of the gene therapy vector. In a further particular embodiment, the one or more immunosuppressants is sirolimus, which is provided to the subject at a dosage that achieves a blood level of sirolimus of 10 ng/ml to 15 ng/ml for a period of time over the sixth months immediately following administration of the gene therapy vector.

In another embodiment, the one or more immunosuppressants comprises mycophenolate mofetil, which is provided to the subject for up to the two months or about the two months immediately following administration of the gene therapy vector. In certain embodiments, the mycophenolate mofetil is provided to the subject at a dosage of 200 mg/m²/day-1200 mg/m²/day or about 600 mg/m²/day.

In another embodiment, the method further comprises providing to the subject prednisolone for a duration of time of between the three to ten days, about the five days, about the six days, or about the seven days immediately following administration of the gene therapy vector. In particular embodiments, the predinsolone is provided to the subject at a dosage of about 0.2 mg/kg/day to about 5 mg/kg/day or about 1 mg/kg/day.

In particular methods, the subject is provided with: (1) prednisolone at a dose of about 1 mg/kg/day beginning within twelve hours before or after administration of the gene therapy vector and continuing for about 10 days; (2) tacrolimus at a starting dose of about 0.2 mg/kg/day (e.g., given in two divided doses) beginning 14 days before administration of the gene therapy vector and adapted after seven days of treatment to target a residual dose of 10-15 ng/ml during the first three months and 7-10 ng/ml from the fourth month and up to at least one year following administration of the gene therapy vector; and (3) mycophenolate mofetil at a dosage of 600 mg/m2 (maximum 2) twice daily, beginning 14 days before administration of the gene therapy vector and maintained for two months or eight weeks following administration of the gene therapy vector.

In particular methods, the subject is provided with: (1) prednisolone at a dose of about 1 mg/kg/day beginning within twelve hours before or after administration of the gene therapy vector and continuing for about 10 days; (2) sirolimus at a dosage that achieves a blood level of sirolimus of 10 ng/ml-15 ng/ml for a period of time over the sixth months immediately following administration of the gene therapy vector; and (3) mycophenolate mofetil at a dosage of 600 mg/m2 (maximum 2) twice daily, beginning 14 days before administration of the gene therapy vector and maintained for two months or eight weeks following administration of the gene therapy vector.

In another particular embodiment, the present disclosure includes a method of treating MPSIIIA, said method comprising administering to a subject in need thereof (e.g., a human diagnosed with MPSIIIA):
(1) a composition comprising a viral vector comprising an expression cassette and flanking sequences comprising the following, in 5′ to 3′ order, wherein the CAG promoter sequence is operably linked to the polynucleotide sequence encoding SGSH:
   a. an AAV2 ITR sequence;
   b. a CAG promoter sequence;
      a polynucleotide sequence encoding a human N-sulfoglucosamine sulfohydrolase (SGSH) polypeptide or an active variant thereof
   c. a human growth hormone polyA sequence; and
   d. an AAV2 ITR sequence,
      wherein said composition is administered via intracerebral injection to twelve sites within the subject's brain through six burr holes in the subject's head, wherein the composition is administered to one or two sites through each burr hole or track.,

In some embodiments, the injection sites are selected from the group consisting of anterior right superficial, anterior right deep, anterior left superficial, anterior left deep, medial right superficial, medial right deep, medial left superficial, medial left deep, posterior right superficial, posterior right deep, posterior left superficial, and posterior left deep, wherein the total amount of composition administered to said subject is about 5×10¹⁰ gc to about 5×10¹³ gc, about 1.0×10¹¹ gc to about 1.0×10¹³ gc, about 1.0×10¹⁰ gc to about 1.0×10¹⁴ gc, about 5.0×10¹⁰ gc to about 5.0×10¹³ gc, about 5.0×10¹⁰ gc to about 1.0×10¹³ gc, about 1.0×10¹¹ gc to about 1.0×10¹³ gc, about 1.0×10¹¹ gc to about 5.0×10¹² gc, or about 5.0×10¹¹ gc to about 5.0×10¹² gc, wherein about 5.0×10⁹ gc to about 5.0×10¹² gc, about 1.0×10¹⁰ gc to about 1.0×10¹² gc, about 0.8×10⁹ gc to about 0.8×10¹³ gc, about 0.4×10¹⁰ gc to about 0.4×10¹³ gc, about 0.4×10¹⁰ gc to about 0.8×10¹² gc, about 0.8×10¹⁰ gc to about 0.8×10¹² gc, about 0.8×10¹⁰ gc to about 0.4×10¹² gc, or about 0.4×10¹¹ gc to about 0.4×10¹² gc or about 6×10¹⁰ gc or about 1.0×10¹² gc or about 1.2×10¹² gc or about 1.4×10¹² gc of viral vector is administered to each of the twelve sites, wherein the volume of composition administered to each of the twelve sites is about 10 μl to about 600 μl, wherein the infusion rate for administration of the vector is about 0.1 μl/min to about 10 μl/min, about 0.2 μl/min to about 8 μl/min, about 0.3 μl/min to about 6 μl/min, or about 0.4 μl/min to about 4.0 μl/min, about 0.4 μl/min to about 3.0 μl/min or about 2.0 μl/min or about 5.0 μl/min; and
(2) one or more immunosuppressants,
   wherein said one or more immunosuppressants comprise or consist of: prednisolone at a dose of about 1 mg/kg/day beginning within twelve hours before or after administration of the gene therapy vector and continuing for about 10 days; tacrolimus at a starting dose of about 0.2 mg/kg/day (e.g., given in two divided doses) beginning 14 days before administration of the gene therapy vector and adapted after seven days of treatment to target a residual dose of 10-15 ng/ml during the first three months and 7-10 ng/ml from the fourth month and up to at least one year following administration of the gene therapy vector; and mycophenolate mofetil at a dosage of 600 mg/m2 (maximum 2) twice daily, beginning 14 days before administration of the gene therapy vector and maintained for two months or eight weeks following administration of the gene therapy vector.

The skilled artisan will appreciate that the above methods and advantages associated with coadministration of one or more immunosuppressants may be adapted for the treatment of other genetic disease or disorders by gene therapy using a vector that administers a functioning copy of a mutated or absent gene product.

Thus, the present disclosure further comprises a method of treating a disease or disorder caused by a mutated or deleted gene in a subject in need thereof, said method comprising:
(1) administering to the subject a gene therapy vector comprising an expression cassette comprising a polynucleotide sequence encoding the polypeptide encoded by the gene in its wild-type or non-mutated form, or an active variant thereof, wherein said polynucleotide sequence is operably linked to a promoter sequence; and
(2) providing to the subject one or more immunosuppressants, wherein at least one of the immunosuppressant is provided to the subject for a duration of time of at least the two months, at least the three months, at least the four months, at least the five months, at least the six months, at least the seven months, at least the eight months, at least the ten months, or at least the twelve months immediately following administration of the gene therapy vector.

In particular embodiments, the one or more immunosuppressants comprises a calcineurin inhibitor, optionally tacrolimus, or mycophenolate mofetil, and/or a macrolide, e.g. sirolimus or rapamicyn.

In further related embodiment, the present disclosure includes a method of treating a brain disorder resulting from a mutated gene in a subject in need thereof, comprising: administering to the subject a gene therapy vector comprising an expression cassette comprising a polynucleotide sequence encoding the polypeptide encoded by the gene in its wild-type or non-mutated form, or an active variant thereof, wherein said polynucleotide sequence is operably linked to a promoter sequence; and providing to the subject one or more immunosuppressants, wherein at least one of the immunosuppressant is provided to the subject for a duration of time of at least the two months immediately following administration of the gene therapy vector.

In particular embodiments, the one or more immunosuppressants comprises any of those or any combination of those described herein, including any dosing regimen described herein in the context of administration of the immunosuppressant in combination with a gene therapy vector for delivery of the SGSH polypeptide. In one embodiment, the one or more immunosuppressants comprises a calcineurin inhibitor, optionally tacrolimus, and mycophenolate mofetil. In another embodiment, the one or more immunosuppressants comprises a macrolide, optionally sirolimus, and mycophenolate mofetil. In particular embodiments, the calcineurin inhibitor or macrolide is provided to the subject for a duration of time of at least the three months, at least the six months, or at least the twelve months immediately following administration of the gene therapy vector.

In certain embodiments wherein the calcineurin inhibitor is tacrolimus, it is provided to the subject at a dosage of 0.05 mg/kg/day-1.0 mg/kg/day or about 0.2 mg/kg/day for at least the two months or at least the three months immediately following administration of the gene therapy vector. In certain embodiments wherein the calcineurin inhibitor is tacrolimus, it is provided to the subject at a dosage that achieves a blood concentration of tacrolimus of 10 ng/ml-15 ng/ml for a period of time during the three months immediately following administration of the gene therapy vector. In certain embodiment wherein the calcineurin inhibitor is tacrolimus, it is provided to the subject at a dosage that achieves a blood concentration of tacrolimus of 7 ng/ml-10 ng/ml for a period of time during the fourth month through the twelfth month immediately following administration of the gene therapy vector.

In certain embodiments wherein the macrolide is sirolimus, it is provided to the subject at a dosage of 3 mg/kg/day or about 1.5 mg/kg/twice per day for at least the two months or at least the three months immediately following administration of the gene therapy vector. In particular embodiments wherein the macrolide is sirolimus, it is provided to the subject at a dosage that achieves a blood level of sirolimus of 10 ng/ml-25 ng/ml for a period of time during the three months immediately following administration of the gene therapy vector. In certain embodiments, wherein the macrolide is sirolimus, it is provided to the subject at a dosage that achieves a blood level of sirolimus of 20 ng/ml for a period of time during the second month through the sixth month immediately following administration of the gene therapy vector. In a further particular embodiments wherein the macrolide is sirolimus, it is provided to the subject at a dosage that achieves a blood level of sirolimus of 10 ng/ml-15 ng/ml for a period of time over the sixth months immediately following administration of the gene therapy vector.

In certain embodiments wherein the one or more immunosuppressant comprises mycophenolate mofetil, it is provided to the subject for up to the two months or about the two months immediately following administration of the gene therapy vector. In particular embodiments, the mycophenolate mofetil is provided to the subject at a dosage of 200 mg/m²/day-1200 mg/m²/day or about 600 mg/m²/day.

In particular embodiments, the method further comprises providing to the subject prednisolone for a duration of time of between the three to ten days, about the five days, about the six days, or about the seven days immediately following administration of the gene therapy vector.

In addition, any of the specific features of the methods described above with respect to treatment of MPSIIIA may be present in the method related more generally to the use of any gene therapy vector, including but not limited to specific immunosuppressants or combinations thereof and specific dosages.

These methods of the present disclosure may be used to treat any genetic disease or disorder, including those caused by a defect in one or more genes, such as, e.g., a gene mutation or deletion, using a gene therapy vector in combination with one or more immunosuppressants. The genetic basis for a large number of disorders and diseases is known. In certain embodiments, a method of the present disclosure is used to treat a lysosomal storage disease or disorder, including any of those described above. Lysosomal storage diseases and disorders that may be treated according to methods of the present disclosure include, but are not limited to: Activator Deficiency/GM2 Gangliosidosis; Alpha-mannosidosis; Aspartylglucosaminuria; Cholesteryl ester storage disease; Chronic Hexosaminidase A Deficiency; Cystinosis; Danon disease; Fabry disease; Farber disease; Fucosidosis; Galactosialidosis; Gaucher Disease (Type I, Type II, Type III); GM1 gangliosidosis (Infantile, Late infantile/Juvenile. Adult/Chronic); I-Cell disease/Mucolipidosis II; Infantile Free Sialic Acid Storage Disease/ISSD; Juvenile Hexosaminidase A Deficiency; Krabbe disease (Infantile Onset, Late Onset); Metachromatic Leukodystrophy; Mucopolysaccharidoses disorders (Pseudo-Hurler polydystrophy/Mucolipidosis IIIA; MPSI; MPSI Scheie Syndrome; MPS I Hurler-Scheie Syndrome; MPS II Hunter syndrome; Sanfilippo syndrome Type A/MPS III A; Sanfilippo syndrome Type B/MPS III B; Sanfilippo syndrome Type C/MPS III C; Sanfilippo syndrome Type D/MPS III D; Morquio Type A/MPS IVA; Morquio Type B/MPS IVB; MPS IX Hyaluronidase Deficiency; MPS VI Maroteaux-Lamy; MPS VII Sly Syndrome; Mucolipidosis I/Sialidosis; Mucolipidosis IIIC; Mucolipidosis type IV); Multiple sulfatase deficiency; Niemann-Pick Disease (Type A, Type B, Type C); Neuronal Ceroid Lipofuscinoses (CLN6 disease (Atypical Late Infantile, Late Onset variant, Early Juvenile), Batten-Spielmeyer-Vogt/Juvenile NCL/CLN3 disease, Finnish Variant Late Infantile CLN5, Jansky-Bielschowsky disease/Late infantile CLN2/TPP1 Disease, Kufs/Adult-onset NCL/CLN4 disease, Northern Epilepsy/variant late infantile CLN8, Santavuori-Haltia/Infantile CLN1/PPT disease, Beta-mannosidosis); Pompe disease/Glycogen storage disease type II; Pycnodysostosis; Sandhoff disease/Adult Onset/GM2 Gangliosidosis; Sandhoff disease/GM2 gangliosidosis—Infantile; Sandhoff disease/GM2 gangliosidosis—Juvenile; Schindler disease; Salla disease/Sialic Acid Storage Disease; Tay-Sachs/GM2 gangliosidosis; and Wolman disease.

In other embodiments, the present disclosure includes an association of:
(1) a gene therapy vector comprising an expression cassette comprising a polynucleotide sequence encoding the polypeptide encoded by the gene in its wild-type or non-mutated form, or an active variant thereof, wherein said polynucleotide sequence is operably linked to a promoter sequence; and
(2) one or more immunosuppressant compounds, for use as a medicament of a brain disorder resulting from a mutated gene.

In particular embodiments of the association, the gene therapy vector and the immunosuppressant are administered simultaneously or subsequently. In certain embodiments, the gene therapy vector is administered by intracerebral injection.

In certain embodiments of the association, at least one of the immunosuppressant compound is provided to the subject for a duration of time of at least the two months immediately following administration of the gene therapy vector.

In certain embodiments of the association, the one or more immunosuppressant compound is selected from the group comprising a calcineurin inhibitor, a macrolide, and mycophenolate mofetil. In particular embodiments, the calcineurin inhibitor is tacrolimus. In other embodiments, the macrolide is sirolimus. In certain embodiments, at least one of the immunosuppressant compound is provided to the subject for a duration of time of at least the three months, at least the six months, or at least the twelve months immediately following administration of the gene therapy vector. In particular embodiments, this compound is the calcineurin inhibitor or the macrolide.

In other embodiments, one or more immunosuppressant compound is also provided to the subject for a duration of time prior to administration of the gene therapy vector.

In exemplary embodiments, the calcineurin inhibitor is tacrolimus, which is provided at a dosage of 0.05 mg/kg/day-1.0 mg/kg/day or about 0.2 mg/kg/day for at least the two months or at least the three months immediately following administration of the gene therapy vector. In related embodiments, the calcineurin inhibitor is tacrolimus, which is provided to the subject at a dosage that achieves a serum level of tacrolimus of 10 ng/ml-15 ng/ml for a period of time during the three months immediately following administration of the gene therapy vector. In further related embodiments, the calcineurin inhibitor is tacrolimus, which is provided to the subject at a dosage that achieves a serum level of tacrolimus of 7 ng/ml-10 ng/ml for a period of time during the fourth month through the twelfth month immediately following administration of the gene therapy vector.

In particular embodiments of an association, the one or more immunosuppressant comprises mycophenolate mofetil, which is provided to the subject for up to the two months or about the two months immediately following administration of the gene therapy vector. In particular embodiments, the mycophenolate mofetil is provided at a dosage of 200 mg/m²/day-1200 mg/m²/day

In other embodiments of an association, the association further comprise prednisolone.

In particular embodiments of an association, the brain disorder resulting from a mutated gene is Sanfilippo type A syndrome.

In various embodiments of an association, the gene therapy vector is a replication deficient adeno-associated virus (AAV)-derived vector comprising an expression cassette comprising in the following 5′ to 3′order:

a promoter sequence;

a polynucleotide sequence encoding a human N-sulfoglucosamine sulfohydrolase (SGSH)polypeptide or an active variant thereof;
and

a polyadenylation (polyA) sequence.

In particular embodiments, the promoter sequence is derived from a CAG promoter sequence; and/or

the polyA sequence is derived from a human growth hormone polyA sequence.

In certain embodiments of an association, the expression cassette comprises in the following 5′ to 3′order:

a promoter sequence derived from a CAG promoter sequence;

a polynucleotide sequence encoding a human N-sulfoglucosamine sulfohydrolase (SGSH) polypeptide or an active variant thereof, and

a polyA sequence derived from a human growth hormone polyA sequence.

In particular embodiments of any of these associations, the expression cassette is flanked by two AAV2 internal terminal repeat (ITR) sequences, wherein one of the two AAV2 ITR sequences is located 5′ of the expression cassette and one of the two AAV2 ITR sequences is located 3′ of the expression cassette. In particular embodiments of any of these associations, the vector further compress an AAVrh.10 capsid, capsid protein, or serotype.

In certain embodiments related to the administration of both a gene therapy vector, including any of those specifically described herein, and one or more immunosuppressants, the gene therapy vector is administered at any of the dosages described herein.

The gene therapy vectors and compositions of the disclosure may be delivered to the brain of a subject through any suitable delivery system known in the art. In certain embodiments, it may be any delivery system that allows the clinician to deliver viral vectors through a needle assembly to targeted sites at depths in the brain with pinpoint precision, e.g., by intracerebral injection. In certain embodiments, the delivery system is designed to be able to reach one or more predetermined depths into the brain tissue, which depths may be precisely and consistently adjusted, e.g., through alterations in the position of a headframe arc, to which a needle assembly is attached, e.g., as described in [55].

In certain embodiments, methods of the present disclosure are performed using a delivery device and procedure as described in Souweidane et al. “Gene therapy for late infantile neuronal ceroid lipofuscinosis: neurosurgical considerations”, J Neurosurg Pediatr. 2010 August; 6 (2):115-22) the entire contents of which are hereby incorporated by reference. As described therein, the gene therapy vector is infused into 12 distinct cerebral locations (2dpths/bur hole, 75 minutes/infusion and 2 μl/minute). Six entry sites, each having 2 depths of injections are used. A first injection or administration may be carried out simultaneously at 6 of the selected locations via six catheters of the delivery system, thereby delivering the composition of the disclosure at this first set of 6 brain locations. Then, each of the 6 catheters may be moved so the six cannulae's proximal ends each reaches the 6 other selected locations. Then, a second injection or administration may be carried out simultaneously at 6 of the selected locations via the six catheters of the delivery system, thereby delivering the composition of the disclosure at this second set of 6 additional brain locations.

As described in Souweidane et al., in particular embodiments, a preoperative imaging and target planning is performed under general anesthesia for a subject, and a preoperative MR imaging of the subject's brain is performed for stereotactic planning. The entry sites may be selected on a BrainLAB workstation (BrainLAB USA), and 3 entry sites over each cerebral hemisphere are selected. The holes may be placed as follows: 1 at each at the frontal pole, the midfrontal region, and the parietal-ocipital region. The frameless navigation and entry site localization is performed. The patient may be registered to preoperative MR image using a minimal of 5 fiducial markers. Preplanned entry sites are marked on the scalp, and a local anesthesic is administered at each entry site. The catheter and gene therapy vector infusion is performed. A 20 gauge spinal needle is used as a rigid guide for insertion of a flexible, fused silica polyimide-coated catheter (inner diameter 150 μm and outer diameter 362 μm (polymicro technology)). Each catheter is connected to a separate syringe, and the catheter is inserted until its tip is flush with the needle tip. The catheter is marked at the top of the needle as a point of interface between the catheter and the brain prior deeper insertion. The spinal needle is affixed to a flexible retractor arm of the head frame and orientated to match the predetermined stereotactic trajectory. Retractor arms and needles are all set to the planned orientation prior to skin opening. The needle is then inserted just below the pial surface and the retractor arm is secured with rigid fixation. The dead space is flush out by running the infusion system. The guide needle is removed, and the catheter is advanced to initial target depth, which is generally 2 cm from the cortical surface with a range of 1.7-2.5 cm. Each catheter is folded with a Steri-strip at the distance from the initial zero mark that would place the tip at the desired depth from the cortical surface. Five minute after cannulation, the gene therapy vector is injected by a microperfusion pump at 2 μl/minutes to each of the 6 sites in parallel. Five minute after completion, the catheter are retracted 1 cm, and a second injection, similar to the first one, is performed. After completion of the second infusion, the catheters are removed.

In another embodiment, the delivery system may be according to the teachings of US 2011/0060285, the entire contents of which are hereby incorporated by reference.

In some embodiments, gene therapy vectors and compositions of the present disclosure may be delivered to the brain of a subject through a cannula. For example, in some embodiments, the gene therapy vectors and compositions are delivered via an MRI Interventions SMARTFLOW® cannula. Use of a controlled flow cannula provides precise, acute infusion at controlled flow rates. In some embodiments, the delivery method comprises the use of a cannula with a guide tube.

### EXAMPLES

### Example 1

### Production of the AAVRH. 10-HMPS3A (SAF-301) Gene Therapy Vector

Clinical grade SAF-301 gene therapy vector was produced under GMP controls by the transfection of two plasmids into a certified 293T human embryonic kidney cell line. DNA coding for the therapeutic gene was provided by one plasmid (pAAV-PGK-hMPS3A) and the capsid proteins (from AAVrh.10), replication genes (from AAV2) and helper functions (from adenovirus serotype 5) were all provided in trans by a second plasmid.

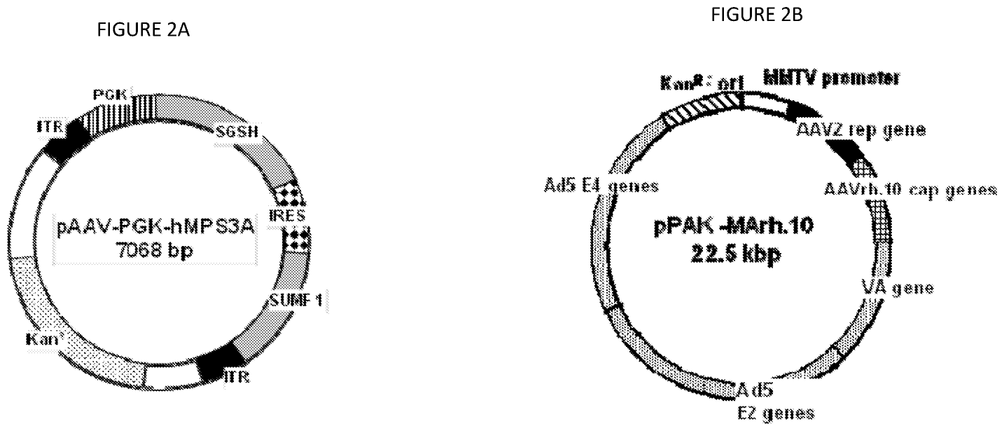

The pAAV-PGK-hMPS3A plasmid was constructed as follows. Commercially available pAAV2-lacZ (7270 bp) was obtained from Stratagene (La Jolla, Calif. USA). The ampicillin resistance gene in the pAAV2-lacZ plasmid was deleted and substituted by a kanamycin resistance gene (KanR, 1119 bp) by subcloning a commercially available kanamycin resistance transposon, creating pAAV2-lacZ (KanR). The expression cassette was subcloned from pAAV-SGSH-IRES-SUMF1 supplied by Alliance Sanfilippo between the inverted terminal repeats of the pAAV2-lacZ (KanR). A diagram of the resulting plasmid is provided in FIG. 2A.

The helper plasmid (pPAK-MArh.10) was based on a kanamycin resistance replicon and contains the VA (nucleotides 9,856 to 11,548 of Genbank M73260), E2A and E4 (nt 21,438-35,935) of Ad serotype 5. The expression of the AAV2 rep gene was driven by a mouse mammary tumor virus (MMTV) promoter (nucleotides 583 to 1325, GenBank AF228552) and the expression of AAVrh.10 cap gene was driven by the endogenous p40 promoter of AAV2. A diagram of this plasmid is provided in FIG. 2B.

After culturing of the transfected cells, the viral vector was released from cells by freeze thaw cycles, purified by an iodixanol step gradient followed by ion exchange chromatography on Hi-Trap QHP columns. The resulting AAVrh.10-hMPS3A vector is transferred into the clinical formulation using concentration by spin column. The purified vector is stored frozen (at or below −60° C.) in phosphate buffered saline.

Full characterization of the final formulated vector was achieved through SDS-PAGE and Western blot for capsid protein, real time PCR for transgene DNA, Western analysis, in vivo and in vitro general and specific adventitious viruses, and enzymatic assay for functional gene transfer. Lot release criteria were provided to assure identity, purity and function.

### Example 2

### In Vivo Efficacy of SAF-301 in Mice

An in vivo study was conducted with SAF-301 to demonstrate the expression of the transgene in the brain. The study design allowed the evaluation of the duration of expression and persistence of the gene after SAF-301 injection and is summarized in Table 3 and described further below.

TABLE 3
Efficacy study summary

| Study name | Adult MPSIIIA mice: clinical study efficacy |
|---|---|
| Objective | To provide pre-clinical data on whether AAV-based |
| | gene therapy is likely to be efficacious and therefore |
| | considered for use in infants and young children |
| | with MPSIIIA |
| | 1) To establish if the AAVrh. 10-hMPS3A is able to |
| | render infected MPSIIIA newborn mouse neural |
| | cells capable of producing active recombinant |
| | human sulfamidase and whether this sulfamidase is |
| | able to catabolise stored HS-derived |
| | oligosaccharides and normalise their level in treated |
| | MPSIIIA. |
| | 2) To clinically, histologically and biochemically |
| | assess the effect of AAVrh.10-hMPS3A on disease |
| | course in live MPSIIIA mice. |
| Species | Mouse MPSIIIA (male and female) |
| Number of | 25 per group |
| animals | |
| Vector/placebo | Tested product: SAF-301: AAVrh 10-hMPS3A |
| | Placebo: AAVrh.10-GFP |
| Experimental | in vivo study |
| design: | animals: 5 weeks mice |
| | dose of vector GFP or SGSH-SUMF1: 7.5 × |
| | 10⁹ gc/animal |
| | route of administration: injection in striatum |
| | number of injection: unilateral injection 2 depths |
| | (2 ×2.5 μl) |
| | euthanasia at 12, 23 and 30 weeks of age |
| | behaviour tests at 15, 18 and 20 weeks of age |
| Studied | 1) SGSH protein level (ELISA) and SGSH |
| parameters | enzymatic activity (HPLC) |
| | 2) Quantification of HS-derived oligosaccharides in |
| | brain |
| | 3) Anti-rhSGSH antibody formation |
| | 4) Histopathology: validated disease markers |
| | (LIMP-2, GFAP, Isolectin B4, Ubiquitin, GM |
| | ganglioside, cholesterol) |
| | 5) Behavioral procedures (hind limb gait, open field |
| | locomotor activity, memory and spatial learning) |
| Conclusion | Delivery of AAVrh.10-hMPS3A to the left striatum |
| | of MPSIIIA mice at 5 weeks of age resulted in the |
| | production of very large quantities (65 × normal) of |
| | (active) SGSH in this brain region. Whilst SGSH |
| | protein (and activity) was also detected in a caudal |
| | region of the injected hemisphere and in the |
| | contralateral hemisphere, particularly in R2, which |
| | is closer to the injection site, the levels were far |
| | lower, and decreased as the distance from the |
| | injection region increased. In contrast to the |
| | outcome of studies in which rhSGSH has been |
| | repeatedly delivered via direct injection into the |
| | cerebrospinal fluid of 6+ week-old mice [5], a |
| | humoral immune response to SGSH was not |
| | detected in mice in this study. |
| | As anticipated, given the regional delivery of the |
| | vector to the brain, AAV-derived SGSH mediated |
| | highly significant but restricted improvements in all |
| | pathological markers examined, at 23 weeks of age. |
| | Although amelioration of some forms of pathology |
| | was noted at the earlier cull time-point (12 weeks of |
| | age; 7 weeks post-injection), it was evident that the |
| | therapeutic effect was not maximal at this point. |
| References | Cressant et al. 2004 |
| | Sondhi et al. 2008 |

In Vivo Efficacy Study

Five week old male and female MPSIIIA mice or unaffected littermates received SAF-301 or a control AAV vector encoding GFP. Each group (n=25) received 7.5×10⁹ vector genome copies in a total of 5 μl. The dose was distributed in two intra-striatal injection sites [29]. Animals were sacrificed at 12, 23 and 30 weeks of age.

Efficacy of transfection and production of the desired enzyme was assessed by evaluation of quantities of SGSH into brain slides and measurement of SGSH activity.

Functional activity of the injection of SAF-301 was evaluated by analysis of the following disease parameters:
(1) the relative level of HS-derived oligosaccharides in brain/spinal cord was determined using tandem mass spectrometry; and
(2) blinded quantitative immunohistological assessments determined the effect of therapy on validated disease markers in fixed MPSIIIA/unaffected brain tissue (LIMP-II, GFAP, Isolectin B4, Ubiquitin, GM3 ganglioside, Unesterified Cholesterol).

Delivery of SAF-301 to the left striatum of MPS IIIA mice at 5 weeks of age resulted in the production of very large quantities (65×normal) of (active) SGSH in this brain region. Whilst SGSH protein (and activity) was also detected in a caudal region of the injected hemisphere and in the contralateral hemisphere, particularly in R2, which is closer to the injection site, the levels were far lower, and decreased as the distance from the injection region increased. A humoral immune response to SGSH was not detected in mice in this study.

Treatment with AAVrh.10-hMPS3A mediated a large but regional reduction in both primary storage material (HS-derived oligosaccharides) and secondary storage products (GM3 and unesterified cholesterol) by 23 weeks of age. Brain slice L2 contains both the striatum (injection region) and the overlying (rostral) cortex in addition to other brain regions we examined, such as the amygdala and substantia innominata. In considering the outcomes from examination of pathological markers contained within left hemisphere slice 2, it was evident that this brain area received sufficient SGSH to mediate a significant (86%) reduction in GlcNS-UA and very large reductions (or normalisation) of GM3 and cholesterol. Further, normalisation of LIMP-II was also observed in structures in this slice (striatum and rostral cortex) and microgliosis was absent/normalised in those areas. Ubiquitin-positive staining was greatly reduced, potentially because lesions were prevented from forming. Astrogliosis due to vector delivery in the left hemisphere of the brain in the region of slice 2 (either AAV-GFP or AAVrh.10-MPS3A) precludes examination of the effect of treatment on this marker in these regions. In slice 2 of the right hemisphere, we observed a 41% reduction in GlcNS-UA and large reductions in GM3 and cholesterol levels but these were not normalised. Similarly, LIMP-II expression was greatly reduced, as was micro- and astrogliosis and ubiquitin-positive staining, but normalisation was not achieved.

Moving caudally, AAVrh.10-MPS3A treatment resulted in a 50% and 21% reduction in GlcNS-UA in left hemisphere and right hemisphere slice 4, respectively. This slice contains the retrosplenial cortex, inferior colliculus and periaqueductal gray, together with other regions not assessed. Our data revealed somewhat mixed effects of the therapy in this more distant area of brain, with reductions in both GM3 and cholesterol observed on the left hemisphere (but not the right hemisphere) of the brain, however, no improvement in LIMP-II, micro- or astrogliosis was seen in either the left or right hemispheres in the inferior colliculus.

The data indicate that this vector is capable of producing sufficient quantities of SGSH in order to mediate highly significant improvements in primary and secondary storage pathology, in addition to downstream events, such as microgliosis. This is particularly evident in the injection region and in adjacent areas. However as observed in the SGSH immune quantification assays, the SGSH activity assays and, as a result, the primary substrate assays (GlcNS-UA), SGSH production by transduced brain cells appears to remain relatively restricted to the injection region, and the impact of it upon pathology becomes less evident when areas more distant from the injection site are examined. It is readily apparent that some regions of brain received insufficient or no SGSH/SUMF1, and thus exhibit little or no improvement in pathological markers. These regions include sites believed to be critical for cognitive function, e.g. dentate gyrus of the hippocampus and retrosplenial cortex. Lack of delivery to these brain regions bilaterally is believed to underlie the failure to improve clinical parameters of disease in MPS IIIA mice.

These findings are in agreement with those of Cearley and Wolfe [10], who reported regional enzyme expression following unilateral AAVrh.10 vector injection into normal mouse brain via two injection tracks (one encompassing rostral cortex and striatum and the other passing through hippocampus to thalamus). The vector used in that study expressed β-glucuronidase and staining of enzyme in successive brain sections illustrated that whilst significant amounts of β-glucuronidase was detectable within the injection hemisphere, the ‘spread’ of enzyme to the contralateral side was far less impressive e.g. ˜55% of cerebral cortex was stained on the injected side, but only 9% was stained on the uninjected hemisphere. The area of contralateral brain receiving the greatest amount of 3-glucuronidase was the thalamus (35% of area stained, compared with 83% stained on injected side).

However, our observations are in direct contrast to those made by Cressant et al [29], who administered an AAV5 vector unilaterally into the striatum and also described restricted vector genome detection, but reported subsequent correction of neuropathology ‘in the entire brain’ and behavioural improvements in MPS IIIB mice. The reason for the disparity in the experimental outcomes is unknown, but may relate to the vector serotype utilized (AAVrh.10 versus AAV5), although this is unlikely if the findings of Sondhi et al [11] are considered. This group examined the extent of distribution of tripeptidyl peptidase I (TPP-I) protein following delivery of a variety of different AAV serotypes to the rat striatum. AAVrh.10 resulted in superior TPP1 distribution, compared with AAV2, AAV5 and AAV8. In order to achieve widespread distribution of TPP1 in the late infantile neuronal ceroid lipofuscinosis model, Sondhi et al subsequently undertook four injections per hemisphere in affected mice. The expression of 1-27×normal levels of TPP-I across the mouse brain permitted reductions in neuropathology in the LINCL model and an improvement (but not normalisation) of clinical disease parameters.

In conclusion, the outcomes of the MPS IIIA mouse study indicated that the AAVrh.10 serotype vector enabled transduced MPS IIIA brain cells (presumptively neurons; [10, 11]), to produce and secrete large quantities of active SGSH, which in turn, mediated highly significant reductions in primary, secondary and other neuropathology. Large quantities of SGSH were detected in some regions of the brain, and appeared to correlate directly with their proximity to the injection site.

### Example 3

### Human Clinical Studies with LYS-SAF301

A human clinical trial was conducted as a phase I/II open-label, single arm, monocentric study of safety assessment, which also provided information on efficacy. In brief, SAF-301 gene therapy vectors (AAVrh.10-hMPS3A, an AAV vector encoding the human SGSH and human SUMF1 gene products) were delivered to both sides of the brain through six image-guided tracks, with two deposits per track, in a single neurosurgical session. Patients received the injections early after diagnosis and the onset of early neurological symptoms, but before severe manifestations. A more detailed description of the clinical trial is provided below.

Gene Therapy Vector

SAF-301 gene therapy vector was prepared as described in Example 1. All production, formulation, and packaging of the investigational agent were in accordance with applicable current Good Manufacturing Practices and met applicable criteria for use in humans. The investigational product was stored at −80° C. before use.

Each patient received 7.2×10¹¹ gc in 12 administrations, each one totaling 60 μl (0.6×10¹¹ gc). The rationale for the proposed clinical dose was based, in part, on clinical trials performed on patients with other types of pathologies, where total doses of AAV vectors ranging from 5.0×10⁹ to 3.2×10¹² vg have been administered without reported toxicity, as well as on the animal studies described herein, which established 0.6×10¹¹ vg/deposit as safe. The volumes used were between 50 and 150 μl and vector concentrations between 0.5 and 1.5×10⁹ vg/μl. Three human gene therapy trials using AAV injection into the brain parenchyma are described in references [32, 33, 34, 35, 36, 37, 38, 39, 40].

Patient Criteria

Four patients were treated.

The main inclusion criteria for the patients included:
(1) age between 18 months and end of six years;
(2) onset of clinical symptoms of MPSIIIA within the first five years of life;
(3) SGSH activity in peripheral blood cells and/or cultured fibroblast extracts less than 10% of controls;
(4) family understanding of the procedure and informed consent; and
(5) vital laboratory parameters within normal range.

Exclusion criteria for the patients included:
(1) presence of brain atrophy on pr-clusion MRI judges on a cortico-dural distance of more than 1 cm;
(2) no independent walking (ability to walk without help);
(3) any condition that would contraindicate permanently anaesthesia;
(4) any other permanent medical condition not related to MPSIIIA;
(5) any vaccination 1 month before investigational drug administration;
(6) receipt of aspirin within one month;
(7) any medication aiming at modifying the natural course of MPSIIIA given during the 6 months before vector injection; and
(8) any condition that would contraindicate treatment with Prograf®, Cellcept® and Solupred®.

Administration Protocol

Vectors were delivered by direct intracerebral injection to both sides of the brain through six image-guided tracks, one deep and one superficial, in a single neurosurgical session. All injections are performed during a single surgery session. Preclinical studies and the published literature indicate that delivery of AAV vectors in a single surgical session is sufficient for efficient gene transfer at all retained sites. The 12 sites were as follows:

TABLE 4
Vector delivery sites

| | | | |
|---|---|---|---|
| Anterior Right | Anterior Right | Anterior Left | Anterior Left |
| Superficial | Deep | Superficial | Deep |
| Medial Right | Medial Right | Medial Left | Medial Left |
| Superficial | Deep | Superficial | Deep |
| Posterior Right | Posterior Right | Posterior Left | Posterior Left |
| Superficial | Deep | Superficial | Deep |

Combination Immunotherapy

The treatment included a combination of the gene therapy described above and the following immunosuppressive treatment.

Apart from the injected vector, the medications used during the trial included:
(1) Tacrolimus (Prograf®) was started 14 days before surgery and was maintained throughout the study. The starting dose was 0.2 mg/kg/day (given in 2 divided doses) and adapted after 7 days of treatment to target a residual dose of 10 to 15 ng/ml during the three first months and 7 to 10 ng/ml from the fourth month and up to at least 1 year;
(2) Mycophenolate mofetil (Cellcept®) was started 14 days before surgery and was maintained for two months at 600 mg/m² (maximum 2 g) twice daily, in oral solution 1 g/5 ml; and
(3) the immunossupressive treatment was associated with prednisolone (Solupred®) during 10 days from the day before surgery

A short pharmacokinetics study of 4 hours (H0, H0.75 and H4) was performed after a week of treatment in order to define the adaptation rules for the dosing.

The day of the surgery, the patients fasted.

The use of an immunosuppressive treatment was based on the results of studies in dog models MPSIIIB and MPSI, where it was shown that the gene therapy necessitated a combination of efficient immunosuppressant treatment to prevent intra- and extra-cerebral immune response against the therapeutic protein and the vector viral antigen.

Following treatment, patients were monitored. One and three months after SAF-301 administration, urine samples were collected and SAF-301 genome titer was measured. GMO detection was performed by qPCR quantifications of DNA specific to the transgene of the SAF-301 vector.

In the initial part of the study, a total of four patients received SAF-301. The drug was well tolerated and no serious adverse reaction or suspected unexpected serious adverse reaction occurred, based on the evaluation of the safety parameters defined in the clinical trial protocol.

Overall, the safety experience from the SAF-301 clinical study has not identified any significant safety risks attributable to SAF-301 in children patients with Sanfilippo type A syndrome. The lack of significant safety findings and signal provides support that the safety profile of SAF-301 is acceptable for use in the treatment of patients with Sanfilippo type A syndrome.

The four patients exhibited a complete elimination of the vector in the urine within 2 to 4 days.

Post-operative clinical assessments did not exhibit any unexplained and sustained fever, seizure or unexpected neurological symptom.

No adverse effects were observed.

The first three patients exhibited the following:
significant decrease in hyperactivity
significant improvement in sleeping patterns
significant improvement in focus and socialization skills.

These results demonstrate the effectiveness of the treatment in human subjects.

### Example 4

### LYS-SAF302 Gene Therapy Vector

LYS-SAF302 is an AAVrh.10 vector that carries a defective AAV2 genome containing the human SGSH gene driven by cytomegalovirus enhancer fused to a chicken β-actin promoter/rabbit β globin intron (CAG promoter). Briefly, vectors were manufactured via triple transient transfection of adherent human embryonic kidney (HEK293) cells. For example, HEK293 cells were costransfected with p-LYS-SAF-T5, pAAV-rh10, and pHGTI and cells were cultured to produce the gene therapy vector. After the cell harvest and lysis steps, the crude viral lysate of rAAV underwent several purification steps, including clarification by depth filtration, affinity chromatography using AVB resin, and tangential flow filtration. LYS-SAF302 was diluted to the target vector concentration in phosphate-buffered saline (PBS) buffer and sterilized by 0.2 μm filtration.

LYS-SAF302 titers were measured using a validated Tagman qPCR method using a forward primer (CCA GCC CCT CCA CAA TGA), a reverse primer (CAC TGG AGT GGC AAC TTC CA) and a probe (CAT CCC TGT GAC CCC).

A schematic representation of the promoter, hSGSH transgene, poly A sequence, and flanking sequences on the LYS-SAF2 plasmid is provided as FIG. 3. A table of the features and the position in SEQ ID NO: 9 for each feature is provided below in Table 5. FIGS. 4A and 4B provide the flanking sequences and the sequence of DNA encapsidated in LYS-SAF302 particles (SEQ ID NO: 9). FIGS. 4C-4D represent the full sequence of the plasmid p-LYS-SAF-T5 (SEQ ID NO: 14), which contains the expression cassette comprising the hSGSH transgene. FIGS. 4E-4F represent the sequence of the plasmid containing the capsid rh10 (pAAV2-rh10; SEQ ID NO: 15) sequence. FIGS. 4G-4K represent the sequence of the helper plasmid pHGTI, i.e., plasmid with helper functions of adenovirus (SEQ ID NO: 16).

TABLE 5
Table of LYS-SAF302 components

| | | Position in SEQ | SEQ ID |
|---|---|---|---|
| Feature | Description | ID NO: 9 | NO |
| L-ITR | Left Inverted terminal | 1-130 | 10 |
| | repeat sequence from AAV | | |
| | serotype 2 | | |
| Promoter | CAG promoter carrying a | 145-1865 | 12 |
| | CMV IE Enhancer, CB | | |
| | promoter, CBA Exon 1, | | |
| | CBA Intron, Rabbit beta- | | |
| | intron, Rabbit beta-globin | | |
| | exon 2 | | |
| Gene of interest | Human SGSH | 1896-3404 | 13 |
| Poly A | Human GH1 poly A | 3417-3926 | 17 |
| R-ITR | Right Inverted terminal | 3935-4075 | 11 |
| | repeat sequence from AAV | | |
| | serotype 2 | | |

The expression cassette comprises, in order, a CMV early enhancer/chicken R actin (CAG) promoter, human N-sulfoglucosamine sulfohydrolase cDNA (hSGSH), and a human growth hormone 1 poly A unit (hGH1 polyA). A first AAV2 inverted repeat (ITR) containing 145 nucleotides and a second AAV2 ITR containing 145 nucleotides flank the expression cassette on either side. The two ITR termini are the only cis-acting elements required for genome replication and packaging. The hGH1 poly A unit is involved in mRNA stability and nuclear export towards mRNA translation. Notably, unlike SAF301, SAF302 does not include an SUMF 1 gene or an IRES sequence. SAF302 also has a different promoter (CAG promoter) relative to SAF301. SAF302 exhibits higher enzyme expression compared to the first generation, SAF301 vector.

LYS-SAF302 DNA consists of 4.07 kb with the sequence molecular weight is of 1257.4 kDa. The SGSH sequence consists of 1.53 kb, and the molecular weight of the SGSH DNA sequence is 471.3 kDa.

### Example 5

### Comparability Study of LYS-SAF301 and LYS-SAF302

Studies were conducted with LYS-SAF302 and LYS-SAF301 to demonstrate and compare the expression and the production of the transgene in the brain and the achievement of functional activity. At 8-14 weeks of age, MPS IIIA mice (6 per group) received bilateral intra-cerebral injections of either vehicle (PBS), LYS-SAF301 or LYS-SAF302 into the caudate putamen/striatum at a dose of 4E+09 vg per animal.

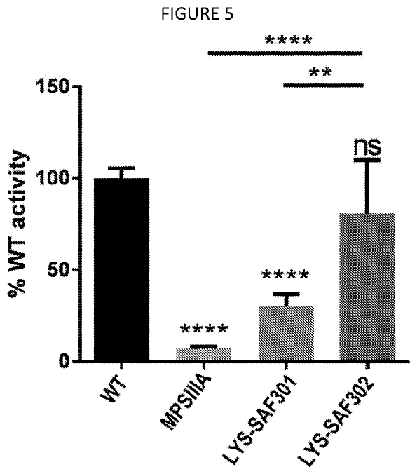

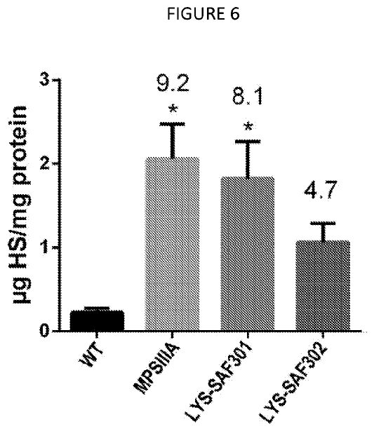

The results showed that LYS-SAF302 provides superior expression compared to LYS-SAF301. Four weeks after injection, LYS-SAF302 led to SGSH enzymatic increase in the brain of injected mice that was 2.7-fold higher than in mice injected with LYS-SAF301 (FIG. 5). A slight reduction in overall heparan sulfate (HS) was seen with LYS-SAF301. In contrast, LYS-SAF302 resulted in a significant drop in HS levels from 9.2-fold WT level in MPS IIIA to 4.7-fold over just 4 weeks postinjection after LYS-SAF302 treatment (FIG. 6). Thus, LYS-SAF302 resulted in significantly higher enzyme expression and a significant drop in HS levels compared to LYS-SAF301.

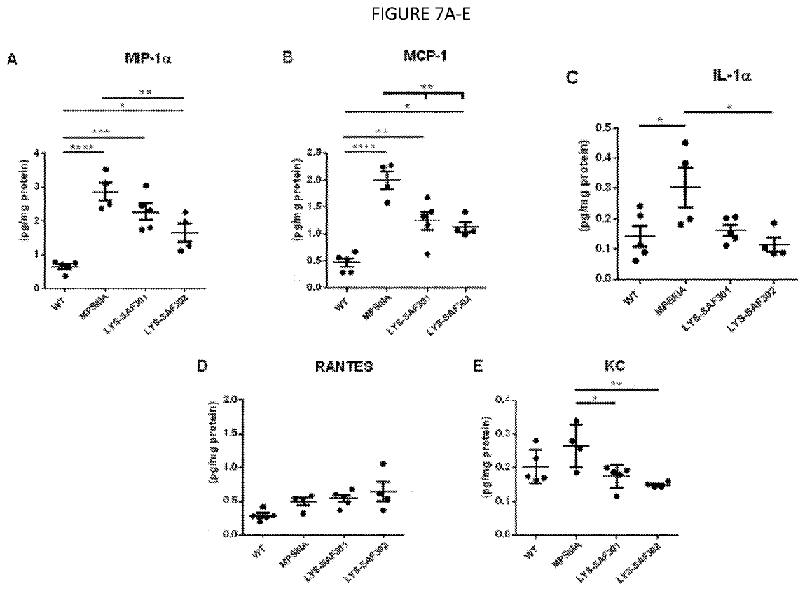

A study was also conducted to assess immunogenicity of LYS-SAF302 as well as compare its immunogenicity to that of LYS-SAF301 The results are provided in FIGS. 7A-7E and show that significant reductions in chemokines and cytokines were observed in LYS-SAF301 and LYS-SAF302 treated mice. MIP-1α, MCP-1 and IL-1α proteins (FIGS. 7A, 7B, and 7C, respectively) were significantly raised in untreated MPS IIIA mice compared to WT. There was a trend towards reduced MIP-1α with each treatment group, however levels were only significantly reduced in LYS-SAF302 treated mice compared to untreated. Significant reductions in MCP-1 were observed with all treatment groups. Levels of IL-1α were reduced with all treatments, yet significant reductions were only observed upon treatment with LYS-SAF302. No significant differences were observed with RANTES and KC (FIGS. 7D and 7E). Neither vector elicited an antibody response after intracortical injections. In summary, the results showed that inflammatory cytokines were reduced by administration of LYS-SAF301 or LYS-SAF302, but LYS-SAF302 exhibited more significant reductions in certain cytokines.

Taken together, the pharmacodynamics study comparing LYS-SAF301 to LYS-SAF302 confirmed that the second-generation product, LYS-SAF302, exhibits higher enzyme expression and better reduction of pathogenic substrate and inflammatory markers compared to the first-generation product LYS-SAF301, with a comparable immunogenicity profile between the two.

### Example 6

### Pharmacology of LYS-SAF302

A dual expression/potency (function) assay based on a semi-quantitative western-blot and an enzymatic assay was developed to assess the in vitro expression and potency of the LYS-SAF302 vector. First, HeLa cells (seeded in 12 well-plate format) were transduced with rAAV2/rh10 vector LYS-SAF302 at 50,000; 100,000; and 500,000 MOI (Multiplicity of Infection). The amount of vector for cell infection was calculated based on the following formula;

Vector volume in μL(V)=(MOI×number of cells per well×1000)/(Vector titer in vg/mL)

About 72 hours post-transduction, Hela cells were collected from the culture plate and centrifuged. The cell pellets were treated with lysis buffer and a freeze thaw cycle was applied to extract the proteins from the cells. The cell suspension was centrifuged and the supernatant was transferred to a new tube.

These samples were then transferred to −80° C. and subsequently used to detect expression of SGSH by Western Blot Assay and to determine enzymatic activity by Potency Assay. Before Western blot analysis, the total protein content of cell lysates was determined by BCA assay (BiCinchoninic acid assay) as this total protein quantification was used to load a defined quantity of protein on the electrophoresis gel (SDS-Page). A standard curve using known amount of hSGSH was also included to allow the protein expression quantification. After the electrophoretic migration allowing proteins to be separated according to their molecular weight, they were transferred on to a nitrocellulose membrane. Proteins of interest were then revealed using anti-actin and anti-SGSH antibodies and fluorochrome-coupled secondary antibodies. hSGSH protein expression was measured through the fluorescent signal at the molecular weight of approximately 57 kDa and normalized by the fluorescent signal of the actin protein observed at approximately 42 kDa. Expression results were expressed as μg.

The potency was determined by an enzymatic assay developed by Karpova et. al. (1996). Briefly, the SGSH cleaves the N-sulfate of the 2-sulfamino-2-deoxy-dglucopyranosyl residue to generate 4MU-αGlcNH2. The 4MU moiety is then liberated by digestion with α-glycosidase, due to α-glucosaminidase activity on the glucosamine residue. Potency results were expressed in nmol/h/mg.

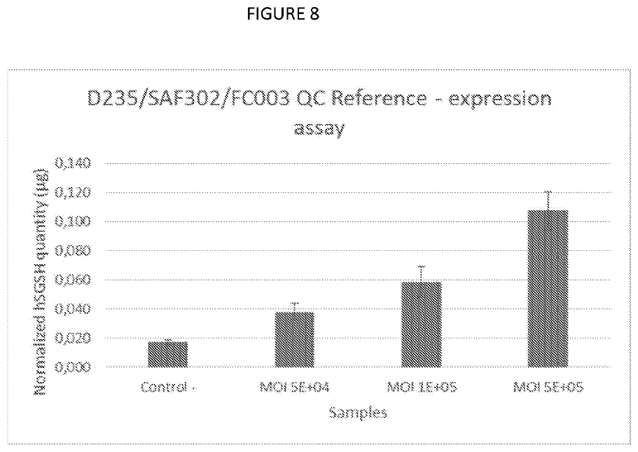

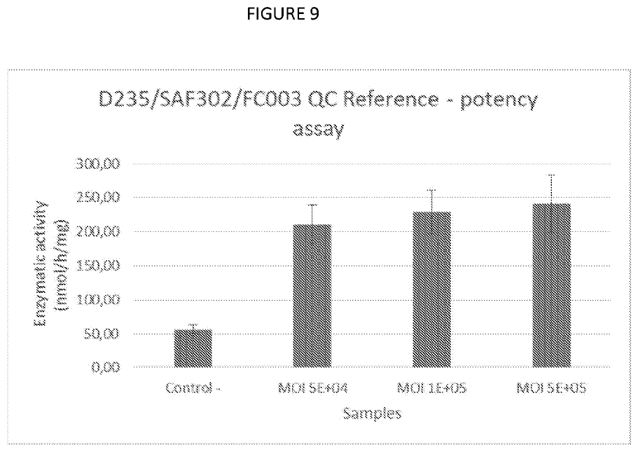

Results of the two assays are provided in FIGS. 8 and 9. An increase was observed between negative control and infected cells. In addition, an expression and slight potency increase was observed with the MOI increase.

### Example 7

### Animal Studies with LYS-SAF302

Efficacy, dose ranging, and toxicology studies of LYS-SAF302 were conducted in MPS IIIA mice, dogs, and non-human primates (NHP).

### Mouse Studies

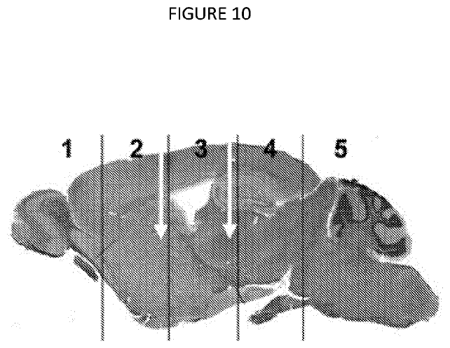

An efficacy/toxicology and dose ranging study of LYS-SAF302 was conducted in 5-week-old MPS IIIA mice. At 5 weeks of age, the MPS IIIA mice (5 per gender per group) received bilateral intra-cerebral injections of either vehicle (PBS) or one of three different doses (8.6E+08, 4.1E+10, and 9.0E+10 vg/animal), of LYS-SAF302 into each of the caudate putamen/striatum and the thalamus. Mice received a total of 8 μL of LYS-SAF302 or vehicle (2 μl per site) at a rate of 0.2 μl/min via 27G needles connected via polyethylene tubing to Hamilton syringes. Bilateral target sites (with respect to bregma) were: posterior aspect of the striatum attempting to include white fiber tracts—A 0.75 mm, L 1.5 mm, V 3 mm and the thalamus—P 2 mm, L 1.5 mm, V 3 mm. Mice received 0.05 mg/kg buprenorphine for pain relief and 4% dextrose in physiological saline for fluid replacement during the procedure. The injection sites and location of the 5 hemi-coronal slices are shown in FIG. 10.

All mice underwent open-field (behavioral) testing at 15 weeks of age. At 17 weeks of age (12 weeks post-surgery), pre-determined cohorts of mice were euthanized and a full post-mortem analysis was performed. Remaining mice underwent open-field testing at 28 weeks of age and were euthanized at 30 weeks of age (25 weeks post-surgery). SGSH activity and HS accumulation were assessed in three brain slices: slice 1 located at the most frontal part of the brain; slice 3 located near the injection site; and slice 5 containing the cerebellum.

SGSH activity in brain homogenates was measured using the fluorogenic substrate 4-methylumbelliferyl-β-D-N-glucosaminide (4MU-αGlcNS). Results were expressed as pmol/min/mg total protein compared to a 4MU standard curve.

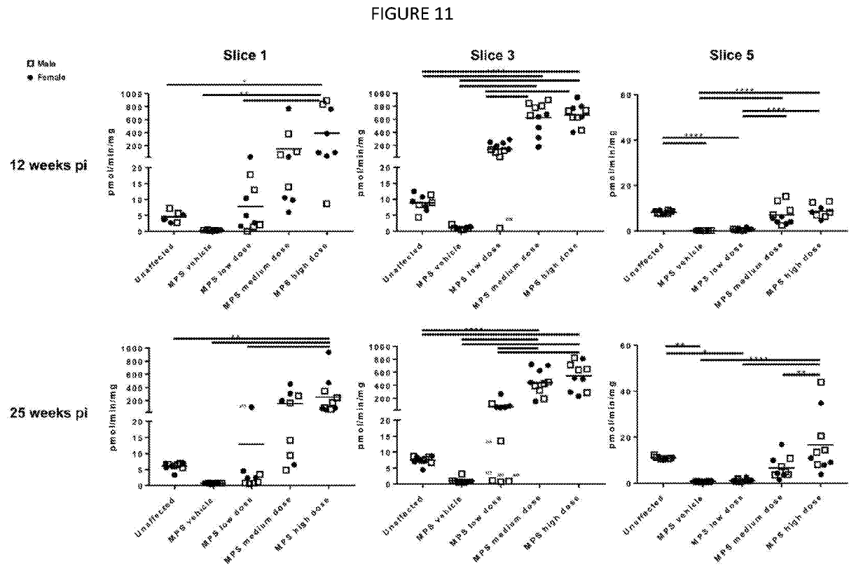

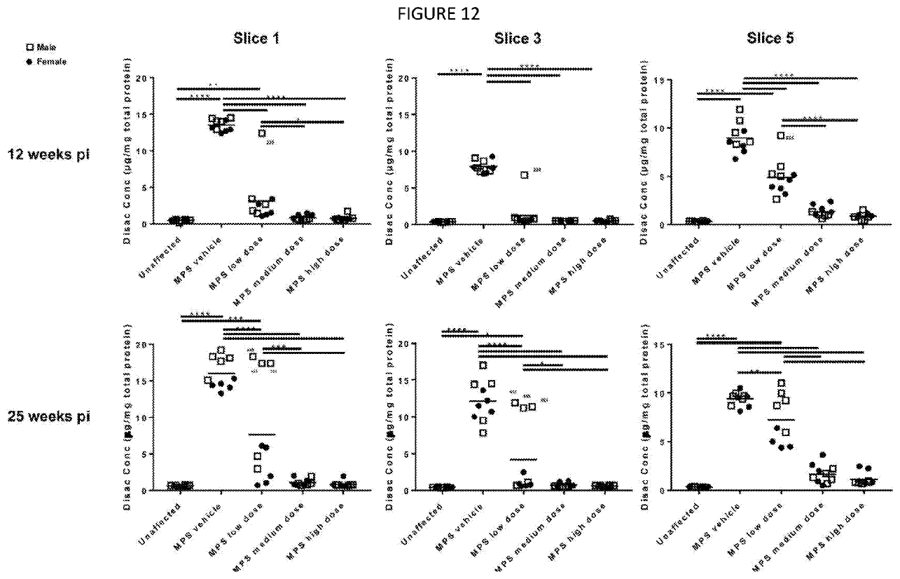

Dose-dependent increase in SGSH activity (FIG. 11) and dose-dependent reductions in primary Heparan sulfate (HS) accumulation (FIG. 12) were noted in the 12 week period following LYS-SAF302 administration that were sustained to 30 weeks of age (25 weeks post-treatment). Globally, higher effects of the treatment were observed near the injection site (slice 3) compared to the most frontal part of the brain (slice 1) or the slice containing the cerebellum (slice 5).

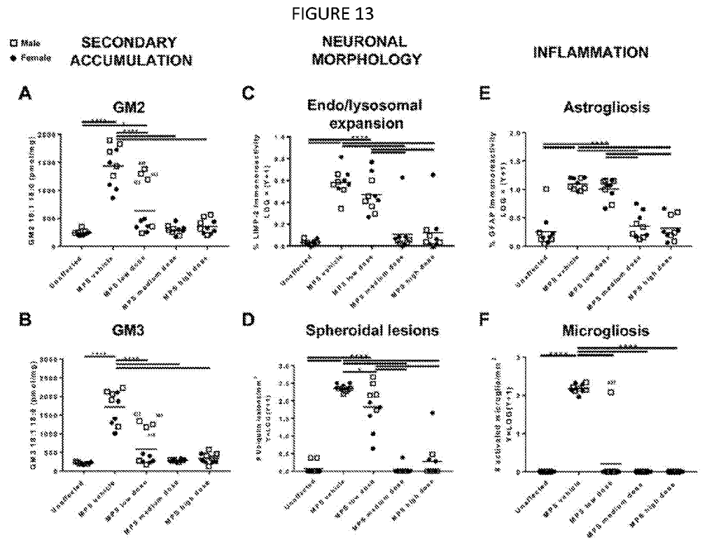

In addition, as shown in FIG. 13A-F, secondary accumulation of GM2/GM3, endo/lysosomal system expansion, astro- and micro-gliosis and resolution of axonal spheroids were noted in the 12 week period following LYS-SAF302 administration that were sustained to 30 weeks of age (25 weeks post-treatment). Secondary accumulation of gangliosides has been reported in several of the MPS diseases, including MPS IIIA 21. GM2 and GM3 was measured in brain slices 1, 3 and 5 (as provided in FIG. 10) at 12 weeks and 25 weeks after treatment (data of slice 3 at 25 weeks post injection presented in FIGS. 13A and 13B. All vehicle-treated MPS IIIA mice exhibited significant increases in GM2/3 in all three brain slices, an outcome observable at each of the two euthanasia ages. There appeared to be no impact of gender on ganglioside levels in any of the vehicle-treated or LYS-SAF302-treated cohorts. Each of the three doses of LYS-SAF302 resulted in normalisation of GM2 and GM3 ganglioside levels in brain slice 1 and 3 (FIGS. 13A, 13B). Whilst ganglioside levels were not normalised in slice 5, there was a reduction in GM2 and GM3 levels post-treatment, particularly notable at the higher two doses.

Characteristic neuronal morphological abnormalities associated with MPS IIIA, consisting of endo/lysosomal expansion and spheroidal lesions were evaluated in several brain areas at 12 weeks and 25 weeks after treatment. Endo/lysosomal expansion reflected by lysosomal integral membrane protein-2 (LIMP2) immunohistochemistry was significantly increased in the vast majority of the MPS IIIA vehicle-treated mouse brain areas examined, in both the 17- and 30-week cull groups (representative data of inferior colliculus at 25 weeks post injection presented in FIG. 13C) Dose-dependent reductions in LIMP2 staining were observed across the rostro-caudal axis of the brain and were maintained to 25-weeks post-treatment, particularly in mice treated with the medium and high doses of LYS-SAF302.

The number of ubiquitin-positive spheroidal lesions >5 μm was evaluated and at both cull times, all regions exhibited a significant increase in ubiquitin-reactive lesions in vehicle-treated MPS IIIA mouse brain compared to age-matched unaffected vehicle-treated mice (representative data of inferior colliculus at 25 weeks post injection presented in FIG. 13D). All LYS-SAF302 doses significantly reduced the number of lesions in all brain areas examined, except the corpus callosum.

Neuroinflammation associated with MPS IIIA was evaluated by the presence of astroglial activation using immunohistochemical detection of glial fibrillary acidic protein (GFAP) and by the presence of activated microglia using histochemical staining of isolectin B4-reactive amoeboid microglia. Significantly increased GFAP expression indicative of astrocyte activation was observed in vehicle-treated MPS IIIA mouse brain regions at both time-points compared to age-matched unaffected vehicle-treated mice, with the exception of dentate gyrus and cerebellum. Representative data for inferior colliculus at 25 weeks post injection are presented in FIG. 13E. A treatment effect was difficult to discern at 17-weeks of age, with no reduction in GFAP expression noted in the caudate, thalamus and the inferior colliculus. Further away from the injection region, there was a reduction in GFAP staining in the brainstem and rostral cortex and inferior colliculus at the early cull time-point. By 30-weeks of age, both levels of the inferior colliculus exhibited significant reductions in GFAP, particularly at the higher two doses (FIG. 13E). However, at this time-point, significantly more GFAP was observed in medium and high dose-treated MPS IIIA mouse rostral cortex (midline) and caudate injection level compared to low dose-treated MPS IIIA mice.

Large numbers of activated microglia were apparent in vehicle-treated MPS IIIA mouse brain compare to vehicle-treated unaffected mouse brain. All three doses essentially resulted in normalisation of microglial morphology, interpreted as deactivation, across the more rostral/central aspects of brain. This outcome was maintained to 30-weeks of age. Representative data for dentate gyrus at 25 weeks post injection are presented in FIG. 13F. Dose-dependent outcomes were noted in brainstem and cerebellum, with the low dose failing to deactivate microglia in the latter, at either timepoint.

The open-field data from female mice at both 15- and 28-weeks of age indicated that female vehicle-treated MPS IIIA mice exhibited the characteristic lower open field activity compared to unaffected vehicle-treated female mice, although the data failed to reach statistical significance, potentially due to under-powering. An improvement in the performance of LYS-SAF302-treated mice was observed, particularly those cohorts receiving the mid and high doses.

At both 15 and 28 weeks of age, male vehicle-treated MPS IIIA mice failed to exhibit the characteristic lower open field activity in this test. Male high-dose treated MPS IIIA mice however, were significantly less active that their unaffected and MPS IIIA vehicle-treated counterparts at 15 weeks of age. By 28 weeks of age, only rearing activity in high dose-treated MPS IIIA males proved to be statistically significantly different compared to unaffected age matched males. The reasons for unusual behavior in male mice are unknown. No difference between males and females were found during the histopathological analysis that can support these behavioral differences.

Taken together, the data from the mouse studies showed that LYS-SAF302 is capable of mediating dose-dependent effects on MPS IIIA-related brain pathology in the timeframe of this experiment (i.e., 25 weeks post administration). This is interpreted as reducing pre-existing disease lesions in some instance (HS accumulation, lysosomal expansion and microgliosis, which would be present at significant levels at the time of dosing). It is also interpreted as preventing the onset of lesion formation in other instances e.g., astrogliosis and axonal spheroid formation, as these types of lesions are slower to develop, and would have been present at lower levels or only in some brain regions at the time of treatment onset. The ability of LYS-SAF302 to significantly reduce microgliosis in the brain of MPS IIIA mice was of particular relevance, as neuroinflammation mediated by activated microglia is thought to play a key role in MPS pathogenesis and disease progression. The ability of LYS-SAF302 to cause a long-lasting reversal of the neuroinflammatory phenotype of microglia, which is thought to lead to and exacerbate neuronal damage in MPS IIIA 23, could have profound implications for its therapeutic potential.

### Dog Studies

Non-GLP dog biodistribution studies were conducted in which various injection parameters were evaluated. The purpose of these studies was to evaluate the effect of various infusion volumes and rates on distribution in adult and juvenile brain using the MRI Interventions SmartFlow cannula.

In one study performed in adult beagle dogs, LYS-SAF302 was co-infused into the white matter with an MRI contrast agent, gadolinium (2-5 mmol), at 1.0E+12 vg/mL and injection speed of 10 μL/min. Four animals received one injection of 500 μL per hemisphere (total dose 1.0E+12 vg/mL) and one animal received two injections of 500 μL per hemisphere (total dose 2.0E+12 vg/mL). Injections were performed using an infusion flow rate of 10 μL/min. The MRI images taken after the injections were analyzed to quantify the distribution of tracer. All animals survived to the time of scheduled necropsies with no changes in body weight, no clinical finding and there were no macroscopic test article-related findings. Animals were humanely euthanized at 1 week or 4 weeks after injection. The right and left hemispheres of the brain were cut into 4 mm thick slices. The even number slabs were placed in sterile petri dishes and 8 mm biopsy punches (40 per animal) immediately taken and cut in half one half for qPCR and one half for SGSH enzyme analysis (4 weeks endpoint groups).

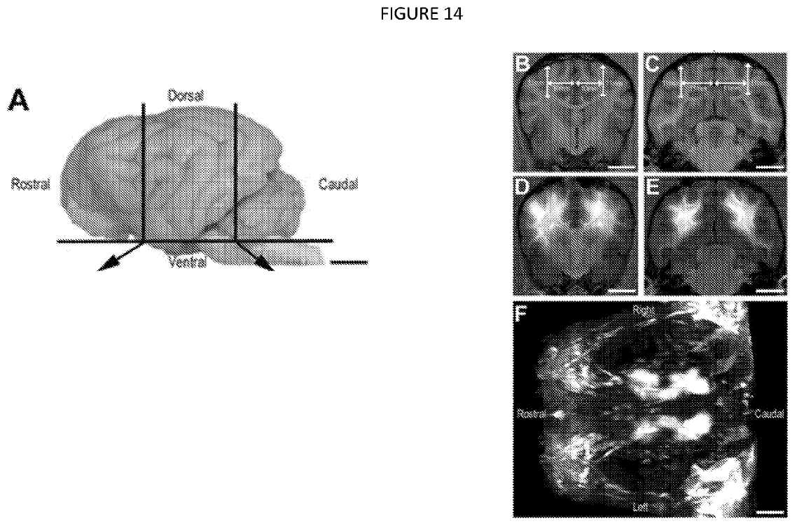

MRI was performed immediately after surgery and collected images were analysed using Osirix software to quantify the gadolinium signal. FIG. 14A is the left lateral view of a dog brain with the position of coronal sections that include sites of injection. The volumes of gadolinium signal per hemisphere were then normalized to the corresponding injection volumes and expressed as the ratio of gadolinium distribution volume/injected volume. FIGS. 14B-C are MRI images of coronal sections before injection with planned site of injection represented with red dot spots. FIGS. 14D-E are MRI images of coronal sections after injection with gadolinium signal visible into the white matter. FIG. 14F is an anterior view of 3D reconstruction of MRI images with gadolinium signal visible in both hemisphere along the rostro-caudal axis of the white matter. Results indicate that the SmartFlow® cannula performed well with no reflux detected. The administration of 500 μL per tract of test article was found to be associated with leakage into the lateral ventricle in both the rostral and caudal injection tracts. The mean ratio gadolinium distribution volume/volume injected was 3.1+/−0.2 (Table 6).

Vector copy analysis was performed by TaqMan qPCR with primers and probe specific for the transgene. A threshold of 0.1 vector copy per cell was set up based on the observation that this (or higher) level was always associated with an SGSH activity increase. At 4 weeks after injection of LYS-SAF302, more than 0.1 vector copy per cell were found in 37% (+/−4) of the brain punches tested (Table 6).

SGSH activity in brain homogenates was measured using the fluorogenic substrate 4-methylumbelliferyl-β-D-N-glucosaminide (4MU-αGlcNS). Results were expressed as % of endogenous activity, determined as the mean value of 80 brain punches from two vehicle injected hemispheres of two distinct animals. Greater than 20% SGSH activity increase was found in 78% (+/−6) of the brain punches tested (Table 6).

TABLE 6
Biodistribution data in dog brain 4 weeks post injection. Data from 6 hemispheres
injected with LYS-SAF302 from 3 dogs with 4 weeks post injection endpoint.

| | Animal | | | | | |
|---|---|---|---|---|---|---|
| | #106 | | #108 | | #109 | |
| Hemisphere | Right | Left | Right | Left | Right | Left |
| Vector concentration (vg/mL) | 1.0E+12 | 1.0E+12 | 1.0E+12 | 1.0E+12 | 1.0E+12 | 1.0E+12 |
| Number (nb) of deposit per hemisphere | 1 | 1 | 1 | 1 | 2 | 2 |
| Volume per deposit (μL) | 500 | 500 | 500 | 500 | 500 | 500 |
| Speed of injection (μL/min) | 10 | 10 | 10 | 10 | 10 | 10 |
| Total dose per hemisphere (vg) | 5.0E+11 | 5.0E+11 | 5.0E+11 | 5.0E+11 | 1.0E+12 | 1.0E+12 |
| Total volume injected (vi) | 0.50 | 0.50 | 0.50 | 0.50 | 1.00 | 1.00 |
| per hemisphere (cm³) | | | | | | |
| Volume of distribution (vd) | 1.56 | 1.43 | 1.50 | 1.39 | 3.63 | 2.92 |
| of gadolinium (cm³) | | | | | | |
| vd gadolinium/vi | 3.1 | 2.9 | 3.0 | 2.8 | 3.6 | 2.9 |
| nb of punches for qPCR analysis | 40 | 40 | 40 | 40 | 33 | 33 |
| nb of punches >0.1 cp/cell | 14 | 15 | 13 | 17 | 10 | 14 |
| % of punches >0.1 cp/cell | 35% | 38% | 33% | 43% | 30% | 42% |
| nb of punches for enzyme analysis | 40 | 40 | 40 | 40 | 33 | 33 |
| nb of punches >20% enzyme increase | 29 | 33 | 30 | 27 | 30 | 26 |
| % of punches >20% enzyme increase | 73% | 83% | 75% | 68% | 91% | 79% |

Taken together, the results indicated that the MRI intervention cannulas performed well with no reflux detected at the injection speed of 10 μL/min. No major difference was found between animals with one injection per hemisphere and the one with two injections per hemisphere. This reflects overlapping of diffusions from the two sites of injection and potential leakage of a part of injected volumes into lateral ventricles due to the very narrow white fiber tracts of approximately 75 cm³ brain. Despite leakage of part of the injected volume into the lateral ventricles, due to the very narrow white fiber tracts of approximately 75 cm³ in dog brain, this study shows that three injections of 500 μL per hemisphere at a vector concentration of 1.0E+12 should be sufficient to cover a large part of a child's brain with the therapeutic levels of at least 20% enzyme activity increase.

An additional pilot study was performed in dogs of 12 weeks old using a lower volume (up to 300 μL) at a reduced injection speed of 5 μL/min. Three (3) animals received one injection per hemisphere (200 μL or 300 μL) of LYS-SAF302 or PBS and 1 animal received two injections per hemisphere (2×200 μL) of LYS-SAF302 in the right hemisphere or PBS in the left hemisphere (total dose of 6E+11 vg to 1.8E+12 vg). The mean ratio gadolinium distribution volume/volume injected was 2.9 (+/−0.5), more than 0.1 vector copy per cell were found in 37% (+/−9) of the brain punches tested and more than 20% SGSH activity increase were found in 53% (+/−8) of the brain punches tested.

### Non-Human Primate (NHP) Studies

Non-human primate (NHP) is also an animal model used to evaluate toxicity and the brain anatomy resembles the human more closely than does dog anatomy. Therefore, NHP was selected specifically for use in the GLP toxicology/biodistribution/dose ranging study to provide a better prediction of the brain distribution and toxicology outcomes in humans. In parallel, a non-GLP study in NHP was performed to provide the neurosurgeons with hands-on experience with the device and to determine the biodistribution of an injection volume and a total dose (relative to the brain volume).

The purpose of these studies was to acquire in the NHP safety and biodistribution data of LYS-SAF302 administered by direct injection into white fiber matter, using the MRI Interventions SmartFlow cannula at injection rate, total volume (related to the brain volume) and total dose (related to brain volume) similar to those to be tested in the pivotal clinical trial. These studies also provided the neurosurgeons with hands-on experience with this new device using clinical surgery settings.

Three (3) male Cynomolgus Monkeys (Macaca fascicularis) (4.2 to 4.5 years old) were dosed with LYS-SAF302 (n=2) or PBS (n=1). LYS-SAF302 was co-infused with the MR contrast agent gadolinium (0.125 mmol/mL), at 3E+12 vg/mL. Four infusions of 50 μL were made in each animal (2 per hemisphere) into the white matter (total dose of 7.2E+11 vg). Vector copy number as well as lysosomal enzyme activity (SGSH) distribution were measured at 6 weeks postinjection.

Vector copy analysis was performed using Tagman qPCR with primers and probe specific for the transgene. A threshold of 0.1 vector copy per cell was set as in the dog study. Six weeks after administration of LYS-SAF302, more than 0.1 vector copy per cell was found in 11% (+/−1) of the brain punches tested (Table 6).

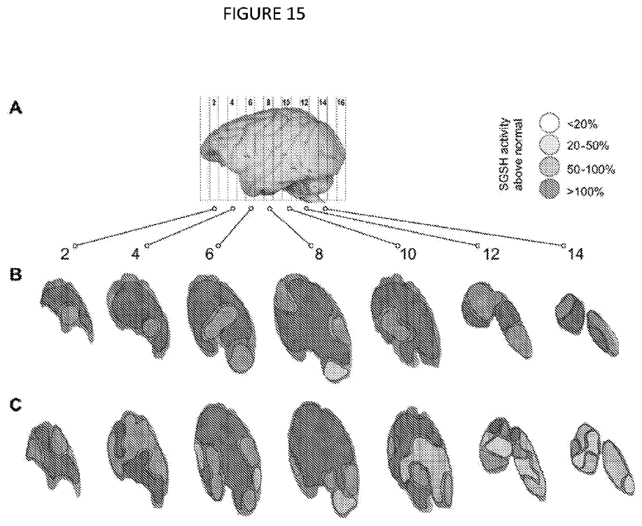

SGSH activity in brain homogenates was measured using 4MU-αGlcNS. Results were expressed as % of endogenous activity, determined as the mean value of 85 punches from the 2 hemispheres of one PBS injected animal. SGSH enzyme activity analysis was also performed and results were expressed as % of endogenous activity. Six weeks after injection, greater than 20% SGSH activity increase was found in 97% (+/−2) of the brain punches tested (Table 6 & FIG. 15).

TABLE 6
Biodistribution data in NHP brain 6 weeks post injection. Data from 4
hemispheres injected with LYS-SAF302 from 2 NHP with 6 weeks post
injection endpoint.

| | Animal | | | |
|---|---|---|---|---|
| | #169I | | #763E | |
| | Hemisphere | | | |
| | Right | Left | Right | Left |
| Vector concentration (vg/mL) | 3.6E+12 | 3.6E+12 | 3.6E+12 | 3.6E+12 |
| Number (nb) of deposit per | 2 | 2 | 2 | 2 |
| hemisphere | | | | |
| Volume per deposit (μL) | 50 | 50 | 50 | 50 |
| Speed of injection (μL/min) | 10 | 10 | 10 | 10 |
| Total dose per hemisphere (vg) | 3.6E+11 | 3.6E+11 | 3.6E+11 | 3.6E+11 |
| nb of punches for qPCR analysis | 46 | 48 | 48 | 43 |
| nb of punches >0.1 cp/cell | 6 | 5 | 4 | 5 |
| % of punches >0.1 cp/cell | 13% | 10% | 8% | 12% |
| nb of punches for enzyme analysis | 51 | 53 | 52 | 50 |
| nb of punches >20% enzyme | 50 | 53 | 50 | 46 |
| increase | | | | |
| % of punches >20% enzyme | 98% | 100% | 96% | 92% |
| increase | | | | |

When normalized to the volume injected, vector diffusion was broader in the NHP study (11% of the brain covered with 100 μL injected) compared to the dog study (37% covered with 500 μL or 1 mL injected), reflecting reduced leakage of injected volumes into the lateral ventricles due to larger white fiber tracts of the NHP brain and lower injected volume. Despite lower absolute levels of vector diffusion in NHP compared to dog, SGSH activity was more broadly distributed throughout the NHP brain (at least 20% activity increase in 97% of the brain vs. 78% in dogs). The differences observed between the two studies could be due to possible species differences or may reflect better enzyme secretion and broad diffusion in the NHP brain after a 6-week period compared to a 4-week period in dogs. Results indicated that the MRI intervention cannulas performed well with no reflux detected at the injection speed of 5 μL/min. While 11% (±1) of the brain punches tested have more than 0.1 vector copy per cell, 97% (±2) of the brain punches tested express more than 20% SGSH activity increase reflecting enzyme secretion and diffusion in all the brain during the 6 week period.

In conclusion, the results of the animal studies validated LYS-SAF302 as a promising new candidate for gene therapy of MPS IIIA. In the phase 1/2 trial conducted with the first generation vector LYS-SAF301 in 4 children with MPS IIIA at a dose of 7.2E+08 vg/g brain, encouraging signs of improvement were noted. The present disclosure provides the second generation vector described herein, which is about 3-fold more potent than the first generation vector, as well as a 10-fold higher clinical dose (7.2E+09 vg/g brain) and using an optimized delivery technique. Without wishing to be bound by theory, the results of the present studies show that a relatively low dose of LYS-SAF302 should be able to restore at least 20% of normal SGSH activity throughout the brain of an MPS IIIA patient. In some embodiments, this level of restoration of enzyme activity has a significant positive impact on disease progression.

### Example 8

### Single Depth Injection

To investigate whether distribution could be enhanced using a larger injection volume at a single depth as opposed to delivery over two depths as in the clinical studies of LYS-SAF301, and to evaluate long-term expression of the LYS-SAF302 vector, a LYS-SAF302 variant was generated in which green fluorescent protein (GFP) replaced the transgene. The vector, SAF302GFP, was identical to LYS-SAF302 except that the hSGSH gene was replaced with the GFP gene. A GFP distribution study was conducted to assess mice 4 months after injection of SAF302GFP.

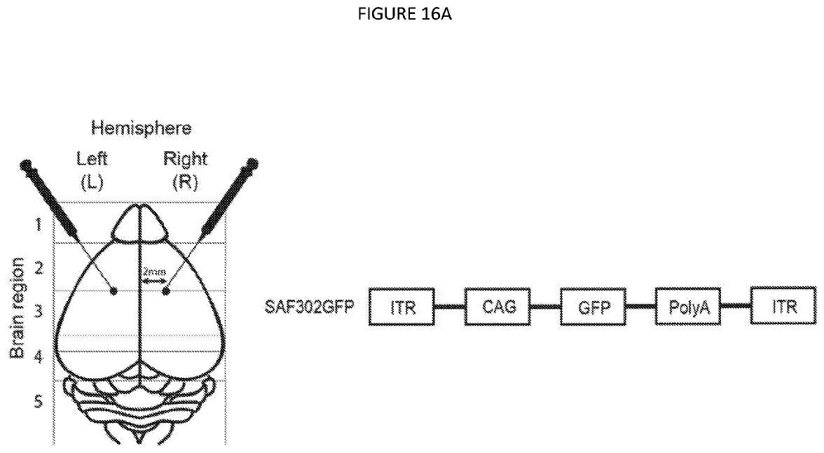

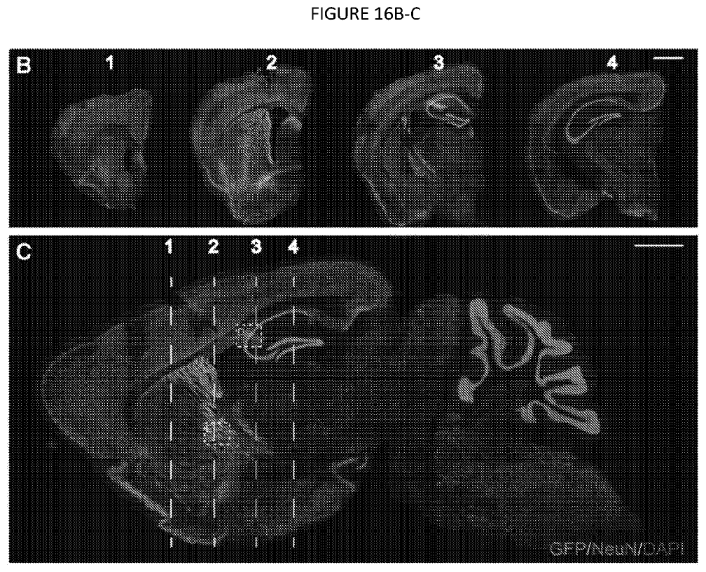

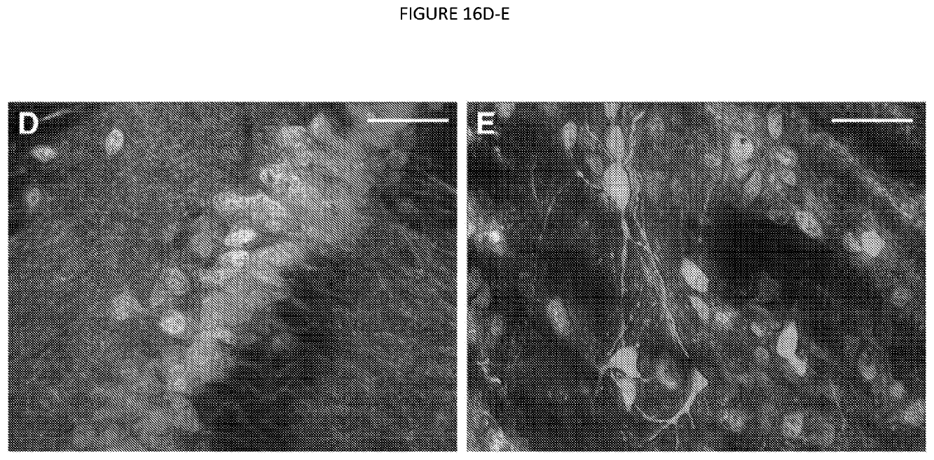

MPSIIIA mice received a stereotactic injection of AAV-SAF302GFP at 8-14 weeks of age via bilateral injections at a single intrastriatal depth, wherein the vector was administered at a dose of 6.1×10⁹ genome particles in 3 μL delivered at the single depth of 3 mm, 2 mm lateral to the midline in both hemispheres (FIG. 16A; n=5). Animals were then sacrificed at approximately 6 months of age, which was around 4 months post injection. For comparison, a two-depth group of MPSIIIA mice received stereotactic injections of AAV-SAF302GFP at 8-14 weeks of age, wherein vectors were administered at a dose of 4.1×10⁹ genome particles in 4 μL, delivered via 1 μL deposits at two intrastratial depths (2 and 3 mm), each 2 mm lateral to the midline in both hemispheres. The mice in the two-depth group were sacrificed at approximately 4 weeks post surgery. Coronal and sagittal sections were co-labeled with NeuN, GFP, and DAPI to give a comprehensive overview of vector distribution throughout the brain

Imaging of the brain sections demonstrated enhanced vector distribution in the animals that received injections at single depth compared to the animals that received injections at two depths. FIGS. 16B-24E show data collected in the single-depth group. Maximal GFP expression was present in the locality of the injection site, reducing in scope and intensity with increasing distance away from this region. Coronal section 2 shows GFP-positive cells throughout the striatum radiating out from the injection site. GFP-positive cells were also visible along the striatum and cortex touching the white matter tract of the external capsule and within the corpus callosum. Expression was considerably greater in coronal section 3 compared to the mice in the two-depth study, with more extensive spread of GFP positive cells within the hippocampus, indicating increased spread of the vector. Internal capsule staining was also apparent, with vector spread apparent in the cerebral peduncle arising from the internal capsule in coronal section 4 (FIG. 16B).

High-magnification images of the hippocampus and the striatum (denoted by the dashed boxes in the sagittal section shown in FIG. 16C) confirmed the neuronal specificity of the SAF302GFP vector (FIGS. 16D and 16E, respectively) with strong colocalization of GFP with neuronal nuclear marker NeuN, with GFP extending along neuronal processes.

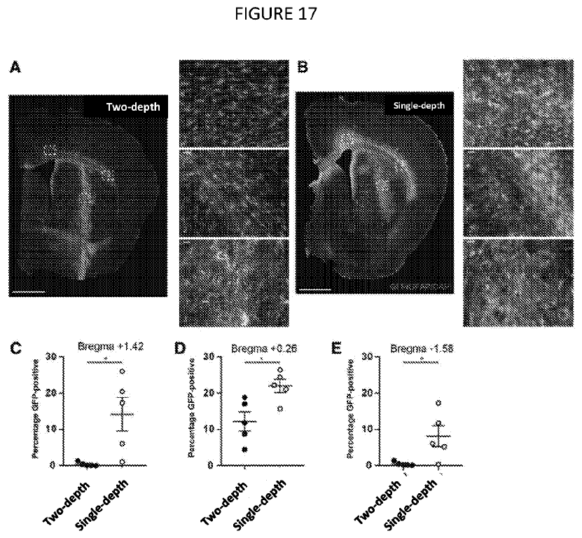

Co-staining of GFP with astrocyte marker GFAP in both two-depth and single-depth animals indicated a proportion of astrocytes were also transduced alongside neurons (FIGS. 17A (two-depth) and 17B (single depth)). However, the proportion of astrocytes targeted was reduced compared to neurons.

No cells within the cerebellum were transduced following intrastriatal injection of SAF302GFP in the single-depth animals, similar to results achieved in the two-depth study arm. Expression of GFP across the bregma following use of the two injection strategies was quantified as the percentage of GFP-positive cells relative to total brain area (FIG. 17C-17E; n=5 animals per group). In the vicinity of the injection site (bregma+0.26), the single injection strategy led to increased global GFP compared to the two-depth strategy (FIG. 17D). Increased transduction of cells distal to the injection site was also apparent with the single bolus injection, whereas little transduction from the double-height injection at distal sites was seen (FIGS. 17C and 17E).

The results of the study indicated that unexpectedly, injecting LYS-SAF302 at two depths was less efficient than injecting at a single depth, despite the reduced volume administered with the single-depth strategy. The vector distributed more along the injection track in the injections at two depths, rather than spreading laterally from the end of the injection site which advantageously occurred in the single injection depth study. Without wishing to be bound by theory, this may reflect reflux of the vector as the needle is withdrawn, rather than creating a larger lateral pressure which can be achieved when delivering a bolus of virus at a single depth.

### Example 9

### Human Clinical Study of SAF-302

A Phase 2/3, single arm, open label, multi-center, interventional study is conducted to assess the safety and efficacy of intracerebral administration of LYS-SAF302 to subject suffering from Sanfilippo type A syndrome. The study includes a 3 year long term follow up. A single dose of 7.2E+12 vg (6 injection sites (3 per hemisphere), 500 μL per injection) is administered to subjects in the supratentorial region of both sides of the brain. LYS-SAF302 is injected through 6 image-guided supratentorial tracks in a single neurosurgical session to target the white matter adjacent to the putamen.

The primary efficacy analysis is performed at 24 months post treatment. An interim analysis is performed at one-year post surgery to identify potential early signal of efficacy. Expected treatment effect is at least a halting of disease progression in a study population selected to have a predictable disease course, based on their age and DQ at baseline. Efficacy is assessed by comparing the observed (post-surgery) evolution of the DQ expressed by the ratio (DQ24/DQ0) between baseline and 24 months and the expected ratio calculated by applying a regression coefficient based on data from 12 patients in the 2 natural history studies (Shapiro et al, 2016 and clinically completed patients from the Phase 1/2 study of SAF301) who met the eligibility criteria for the present study. They will be used to calculate the expected cognitive outcome in the absence of treatment at 12 and 24 months.

For the Phase 2/3 clinical study, clinical grade SAF302 gene therapy vector was produced under GMP controls. The finished product was formulated in PBS buffer without any excipients or preservatives. The solution was clear to slightly opalescent and may contain small whitish to translucent particles. The presence of particles was mitigated via a 0.2-micron inline filter, placed between the syringe and Smartflow cannula during the drug administration procedure, without detectable effect on the strength or potency of drug product LYS-SAF302.

The composition of the PBS buffer was as follows:
2.67 mM KCl
1.47 mM KH2PO4
137.9 mM NaCl
8.06 mM Na2HPO4
pH 7.2-7.4

The product primary packaging for LYS-SAF302 consists of:
Ready-to-use sterile 3 mL (2R) glass vials from Ompi;
Ready-to-sterilize 13 mm FluroTec stoppers from West Pharmaceuticals (steam sterilized by Novasep);
13 mm ready-to-use irradiated flip off caps from West Pharmaceuticals.

The vials are filled with 1.2 mL of LYS-SAF302 at 2.4E+12 vg/mL target vector concentration (release specifications: 1.9E+12 to 3.2E+12 vg/mL).

The therapeutic vector is delivered at a dose of around 7.2E+12 vg/patient (at concentration of around 2.4E+12 vg/mL) in 6 pre-defined (by MRI) simultaneous frameless stereotaxic brain injections (500 μL each, i.e., 1.2E+12 vg) in approximately 100 minutes (5 μL/min), bilaterally within the white matter anterior, medial and posterior to basal ganglia (3 injection sites per hemisphere). Surgery is performed by trained neurosurgeons.

The patients will receive a concomitant immunosuppressive regimen (tacrolimus, mycophenolate mofetil and short-term prednisolone) to avoid elimination of transduced cells. The study shows that LYS-SAF302 delivered via the dosing strategy provided herein results in highly clinically effective treatment of diseases relating to deficiency of SGSH, such as Sanfilippo type A syndrome.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention.

### REFERENCES

1. Valstar, M. J., et al., Sanfilippo syndrome: A mini-review. J Inherit Metab Dis, 2008.
2. Gliddon, B. L. and J. J. Hopwood, Enzyme-replacement therapy from birth delays the development of behavior and learning problems in mucopolysaccharidosis type IIIA mice. Pediatr Res, 2004, 56(1): p. 65-72.
3. Hemsley, K. M., B. King, and J. J. Hopwood, Injection of recombinant human sulfamidase into the CSF via the cerebellomedullary cistern in MPS IIIA mice. Mol Genet Metab, 2007. 90(3): p. 313-28.
4. Hemsley, K. M., et al., Effect of high dose, repeated intra-CSF injection of sulphamidase on neuropathology in MPSIIIA mice. Genes Brain Behav, 2008.
5. Hemsley, K. M., et al., Examination of intravenous and intra-CSF protein delivery for treatment of neurological disease. Eur J Neurosci, 2009. 29(6): p. 1197-214.
6. Savas, P. S., K. M. Hemsley, and J. J. Hopwood, Intracerebral injection of sulfamidase delays neuropathology in murine MPS-IIIA. Mol Genet Metab, 2004. 82(4): p. 273-85.
7. Yu, W. H., et al., Short-term enzyme replacement in the murine model of Sanfilippo syndrome type B. Mol Genet Metab, 2000. 71(4): p. 573-80.
8. Vogler, C., et al., Overcoming the blood-brain barrier with high-dose enzyme replacement therapy in murine mucopolysaccharidosis VII. Proc Natl Acad Sci USA, 2005. 102(41): p. 14777-82.
9. Wu, Z., A. Asokan, and R. J. Samulski, Adeno-associated virus serotypes: vector toolkit for human gene therapy. Mol Ther, 2006. 14(3): p. 316-27.
10. Cearley, C. N. and J. H. Wolfe, Transduction characteristics of adeno-associated virus vectors expressing cap serotypes 7, 8, 9, and Rh10 in the mouse brain. Mol Ther, 2006. 13(3): p. 528-37.
11. Sondhi, D., et al., Enhanced survival of the LINCL mouse following CLN2 gene transfer using the rh.10 rhesus macaque-derived adeno-associated virus vector. Mol Ther, 2007. 15(3): p. 481-91.
12. Klein, R. L., et al., AAV8, 9, Rh10, Rh43 vector gene transfer in the rat brain: effects of serotype, promoter and purification method. Mol Ther, 2008. 16(1): p. 89-96.
13. Fraldi, A., et al., Functional correction of CNS lesions in an MPS-IIIA mouse model by intracerebral AAV-mediated delivery of sulfamidase and SUMF1 genes. Hum Mol Genet, 2007. 16(22): p. 2693-702.
14. Anson, D. S. and J. Bielicki, Sulphamidase. Int J Biochem Cell Biol, 1999. 31(3-4): p. 363-7.
15. Cosma, M. P., et al., The multiple sulfatase deficiency gene encodes an essential and limiting factor for the activity of sulfatases. Cell, 2003. 113(4): p. 445-56.
16. Fraldi, A., et al., SUMF1 enhances sulfatase activities in vivo in five sulfatase deficiencies. Biochem J, 2007. 403(2): p. 305-12.
17. Kurai, T., et al., AAV1 mediated co-expression of formylglycine-generating enzyme and arylsulfatase a efficiently corrects sulfatide storage in a mouse model of metachromatic leukodystrophy. Mol Ther, 2007. 15(1): p. 38-43.
18. Dierks, T., et al., Multiple sulfatase deficiency is caused by mutations in the gene encoding the human C(alpha)-formylglycine generating enzyme. Cell, 2003. 113(4): p. 435-44.
19. Dierks, T., B. Schmidt, and K. von Figura, Conversion of cysteine to formylglycine: a protein modification in the endoplasmic reticulum. Proc Natl Acad Sci USA, 1997. 94(22): p. 11963-8.
20. Dierks, T., et al., Posttranslational formation of formylglycine in prokaryotic sulfatases by modification of either cysteine or serine. J Biol Chem, 1998. 273(40): p. 25560-4.
21. Fey, J., et al., Characterization of posttranslational formylglycine formation by luminal components of the endoplasmic reticulum. J Biol Chem, 2001. 276(50): p. 47021-8.
22. Takakusaki, Y., et al., Coexpression of formylglycine-generating enzyme is essential for synthesis and secretion of functional arylsulfatase A in a mouse model of metachromatic leukodystrophy. Hum Gene Ther, 2005. 16(8): p. 929-36.
23. Adra, C. N., P. H. Boer, and M. W. McBurney, Cloning and expression of the mouse pgk-1 gene and the nucleotide sequence of its promoter. Gene, 1987. 60(1): p. 65-74.
24. Jang, S. K., et al., A segment of the 5′ nontranslated region of encephalomyocarditis virus RNA directs internal entry of ribosomes during in vitro translation. J Virol, 1988. 62(8): p. 2636-43.
25. Kozak, M., Circumstances and mechanisms of inhibition of translation by secondary structure in eucaryotic mRNAs. Mol Cell Biol, 1989. 9(11): p. 5134-42.
26. Vagner, S., B. Galy, and S. Pyronnet, Irresistible IRES. Attracting the translation machinery to internal ribosome entry sites. EMBO Rep, 2001. 2(10): p. 893-8.
27. Samulski, R. J., L. S. Chang, and T. Shenk, A recombinant plasmid from which an infectious adeno-associated virus genome can be excised in vitro and its use to study viral replication. J Virol, 1987. 61(10): p. 3096-101.
28. Mastakov, M. Y., et al., Immunological aspects of recombinant adeno-associated virus delivery to the mammalian brain. J Virol, 2002. 76(16): p. 8446-54.
29. Cressant, A., et al., Improved behavior and neuropathology in the mouse model of Sanfilippo type IIIB disease after adeno-associated virus-mediated gene transfer in the striatum. J Neurosci, 2004. 24(45): p. 10229-39.
30. Arruda, V. R., et al., Lack of germline transmission of vector sequences following systemic administration of recombinant AAV-2 vector in males. Mol Ther, 2001. 4(6): p. 586-92.
31. Schuettrumpf, J., et al., Inadvertent germline transmission of AAV2 vector: findings in a rabbit model correlate with those in a human clinical trial. Mol Ther, 2006. 13(6): p. 1064-73.
32. Janson, C., et al., Clinical protocol. Gene therapy of Canavan disease: AAV-2 vector for neurosurgical delivery of aspartoacylase gene (ASPA) to the human brain. Hum Gene Ther, 2002. 13(11): p. 1391-412.
33. McPhee, S. W., et al., Immune responses to AAV in a phase I study for Canavan disease. J Gene Med, 2006. 8(5): p. 577-88.
34. Crystal, R. G., et al., Clinical protocol. Administration of a replication-deficient adeno-associated virus gene transfer vector expressing the human CLN2 cDNA to the brain of children with late infantile neuronal ceroid lipofuscinosis. Hum Gene Ther, 2004. 15(11): p. 1131-54.
35. Worgall, S., et al., Treatment of late infantile neuronal ceroid lipofuscinosis by CNS administration of a serotype 2 adeno-associated virus expressing CLN2 cDNA. Hum Gene Ther, 2008. 19(5): p. 463-74.
36. Kaplitt, M. G., et al., Safety and tolerability of gene therapy with an adeno-associated virus (AAV) borne GAD gene for Parkinson's disease: an open label, phase I trial. Lancet, 2007. 369(9579): p. 2097-105.
37. Kaplitt, M. G., Parkinson disease: Another player in gene therapy for Parkinson disease. Nat Rev Neurol, 2010. 6(1): p. 7-8.
38. Marks, W. J., Jr., et al., Safety and tolerability of intraputaminal delivery of CERE-120 (adeno-associated virus serotype 2-neurturin) to patients with idiopathic Parkinson's disease: an open-label, phase I trial. Lancet Neurol, 2008. 7(5): p. 400-8.
39. Eberling, J. L., et al., Results from a phase I safety trial of hAADC gene therapy for Parkinson disease. Neurology, 2008. 70(21): p. 1980-3.
40. Christine, C. W., et al., Safety and tolerability of putaminal AADC gene therapy for Parkinson disease. Neurology, 2009. 73(20): p. 1662-9.
41. Jiang, H., et al., Evidence of Multiyear Factor IX Expression by AAV-Mediated Gene Transfer to Skeletal Muscle in an Individual with Severe Hemophilia B. Mol Ther, 2006. 14(3): p. 452.
42. Hopwood, J. J., and Elliott, H. (1982). Diagnosis of Sanfilippo type A syndrome by estimation of sulfamidase activity using a radiolabelled tetrasaccharide substrate. Clin Chim Acta 123, 241-250.
43. King, B., Savas, P., Fuller, M., Hopwood, J., and Hemsley, K. (2006). Validation of a heparan sulfate-derived disaccharide as a marker of accumulation in murine mucopolysaccharidosis type IIIA. Mol Genet Metab 87, 107-112.
44. Crawley, A. C., Gliddon, B. L., Auclair, D., Brodie, S. L., Hirte, C., King, B. M., Fuller, M., Hemsley, K. M., and Hopwood, J. J. (2006). Characterization of a C57BL/6 congenic mouse strain of mucopolysaccharidosis type IIIA. Brain Res 1104, 1-17.
45. McGlynn, R., Dobrenis, K., and Walkley, S. U. (2004). Differential subcellular localization of cholesterol, gangliosides, and glycosaminoglycans in murine models of mucopolysaccharide storage disorders. J Comp Neurol 480, 415-426.
46. Souweidane, M M, Fraser J F, Arkin L M, Sondhi D, Hackett N R, Kaminsky S M, Heier L, Kosofsky B E, Worgall S, Crystal R G, Kaplitt M G. “Gene therapy for late infantile neuronal ceroid lipofuscinosis: neurosurgical considerations” Neurosurg Pediatr. 2010 August; 6(2):115-22.
47. Karpova, E. A., Voznyi, Y. V., Keulemans, J. L. M., Hoogeveen, A. T., Winchester, B., Tsvetkova, I. V., van Diggelen, O. R. (1996) A fluorimetric enzyme assay for the diagnosis of Sanfilippo disease type A (MPS IIIA). Inher. Metab. Dis. 19; p. 278-285.

1. A replication deficient adeno-associated virus serotype rh. 10 (AAVrh.10)-derived vector comprising an expression cassette comprising in the following 5′ to 3′ order:
a. a promoter sequence;
b. a polynucleotide sequence encoding a human N-sulfoglucosamine sulfohydrolase polypeptide or an active variant thereof;
and
c. a polyadenylation (polyA) sequence;
wherein the vector does not include a polynucleotide sequence encoding a human sulfatase modifying factor 1 or any active variant thereof, wherein the promoter comprises the sequence according to SEQ ID NO: 12.

2. (canceled)

3. The vector of claim 1, wherein the polyA sequence is derived from a human growth hormone 1 sequence.

4. The vector of claim 1, wherein the vector does not include an internal ribosomal entry site (IRES) sequence.

5. The vector of claim 1, wherein the expression cassette consists of, in the following 5′ to 3′_order:
a. the promoter sequence derived from a CAG promoter sequence;
b. the polynucleotide sequence encoding a human N-sulfoglucosamine sulfohydrolase polypeptide or an active variant thereof; and
c. the polyA sequence derived from a human growth hormone 1 polyA sequence.

6. The vector of claim 1, wherein the expression cassette is flanked by two AAV2 internal terminal repeat (ITR) sequences, wherein one of the two AAV2 ITR sequences is located 5′ of the expression cassette and one of the two AAV2 ITR sequences is located 3′ of the expression cassette.

7. The vector of claim 6, wherein the ITR sequence located at the 5′ end of the expression cassette comprises the nucleotide sequence according to SEQ ID NO: 10 and the ITR sequence located at the 3′ end of the expression cassette comprises the nucleotide sequence according to SEQ ID NO: 11.

8. (canceled)

9. The vector of claim 1, wherein the polynucleotide sequence encoding a human N-sulfoglucosamine sulfohydrolase comprises the sequence according to SEQ ID NO: 13.

10. The vector of claim 1, wherein the polyadenylation (polyA) sequence comprises the sequence according to SEQ ID NO: 17.

11. The vector of claim 1, wherein said vector further comprises an AAVrh.10 capsid or an AAVrh.10 capsid protein.

12. The vector of claim 1, comprising the following in the following 5′ to 3′ order:
a. an AAV2 ITR sequence;
b. the promoter sequence derived from a CAG promoter sequence;
c. the polynucleotide sequence encoding a human N-sulfoglucosamine sulfohydrolase polypeptide or an active variant thereof;
d. the polyA sequence derived from a human growth hormone 1 polyA sequence; and
e. an AAV ITR sequence.

13. The vector of claim 1, comprising the sequence according to SEQ ID NO: 9.

14. The vector of claim 1, comprising the sequence according to SEQ ID NO: 14.

15. A method of treating Sanfilippo type A syndrome, comprising administering the vector of claim 1 to a subject in need thereof.

16. The method of claim 15, wherein the vector is administered to the subject via intracerebral injection.

17. The method of claim 15, wherein the vector is administered to the subject via intracerebral injections, wherein each injection is administered at a single injection depth.

18. The method of claim 15, wherein the vector is administered to the subject via 2-4 injections per hemisphere.

19. The method of claim 18, wherein the vector is administered to the subject via 3 injections per hemisphere.

20. The method of claim 15, wherein each injection is administered in a volume of about 500 μL.

21. The method of claim 15, wherein the total dose of the vector administered to the subject is between about 5×10¹⁰ to about 5×10¹³ vg.

22. The method of claim 21, wherein the total dose of the vector administered to the subject is about 7.2×10¹² vg.

23. The method of claim 15, wherein the method further comprises administering an immunosuppressive regimen to the subject.

24. The method of claim 23, wherein the immunosuppressive regimen comprises tacrolimus, mycophenolate mofetil, and prednisone.

25. (canceled)

26. A pharmaceutical composition comprising the vector of claim 1 and a pharmaceutically acceptable support, carrier, excipient or diluent.

27. The pharmaceutical composition according to claim 26, wherein the pharmaceutical composition is an emulsion or an aqueous solution.

28. (canceled)
